(19) United States
(12) Patent Application Publication
(10) Pub. No.: US 2008/0076762 A1
(43) Pub. Date: Mar. 27, 2008

(54) Novel 4-Aminopiperidine Derivatives

(75) Inventors: Christoph Boss, Allschwil (CH); Daniel Bur, Therwil (CH); Olivier Corminboeuf, Allschwil (CH); Corinna Grisostomi, Allschwil (CH); Lars Prade, Weil am Rhein (DE); Thomas Weller, Binningen (CH)

Correspondence Address: DICKSTEIN SHAPIRO LLP, 1177 AVENUE OF THE AMERICAS (6TH AVENUE), NEW YORK, NY, 10036-2714, US

(73) Assignee: ACTELION PHARMACEUTICALS LTD., Allschwil (CH)

(21) Appl. No.: 11/720,181

(22) PCT Filed: Nov. 21, 2005
(86) PCT No.: PCT/IB05/53838

§ 371 Date: May 24, 2007

(30) Foreign Application Priority Data
Nov. 25, 2004 (EP) PCT/EP2004/013369

(51) Int. Cl. A61K 31/541; A61K 31/4468; A61K 31/4523; A61P 33/06; C07D 241/04; C07D 295/00; C07D 221/00; A61K 31/496; A61K 31/5377

(52) U.S. Cl. 514/227.8; 514/235.5; 514/253.11; 514/316; 514/318; 514/326; 514/329; 544/129; 544/364; 544/60; 546/187; 546/194; 546/209; 546/213; 546/214; 546/224

(57) ABSTRACT

Novel substituted 4-aminopiperidine derivatives of the formula I: 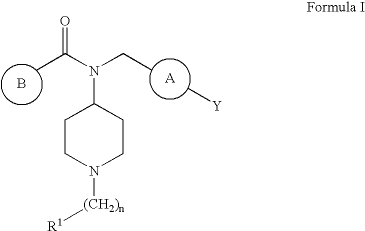
wherein n, R¹, Y, 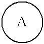
and 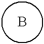
are as defined in claim 1, and optically pure enantiomers, mixtures of enantiomers, racemates, diastereomers, mixtures of diastereomers, diastereomeric racemates, mixtures of diastereomeric racemates and meso-forms, as well as salts and solvent complexes of such compounds, and morphological forms, that exhibit useful parasite aspartic proteases inhibiting properties and can thus be used in the form of pharmaceutical compositions as antimalarial medicines.

### FIELD OF THE INVENTION

The present invention relates to novel 4-aminopiperidine derivatives of the formula I below. The invention also concerns related aspects including pharmaceutical compositions containing one or more compounds of formula I and especially their use as inhibitors of the plasmodium falciparum protease plasmepsin II, the plasmodium falciparum protease plasmepsin IV or related aspartic proteases such as the plasmodium falciparum protease plasmepsin I and HAP (Histoaspartic protease) or other protozoal or fungal aspartic proteases.

### BACKGROUND OF THE INVENTION

Malaria is one of the most serious and complex health problems affecting humanity in the 21^{st} century. The disease affects about 300 million people worldwide, killing 1 to 1.5 million people every year. Malaria is an infectious disease caused by four species of the protozoan parasite Plasmodium, P. falciparum being the most severe of the four. All attempts to develop vaccines against P. falciparum have failed so far. Therefore, therapies and preventive measures against malaria are confined to drugs. However, resistance to many of the currently available antimalarial drugs is spreading rapidly and new drugs are needed.

P. falciparum enters the human body by way of bites of the female anophelino mosquito. The plasmodium parasite initially populates the liver, and during later stages of the infectious cycle reproduces in red blood cells. During this stage, the parasite degrades hemoglobin and uses the degradation products as nutrients for growth [Goldberg, D. E., Slater, A. F., Beavis, R., Chait, B., Cerami, A., Henderson, G. B., Hemoglobin degradation in the human malaria pathogen Plasmodium falciparum: a catabolic pathway initiated by a specific aspartic protease, J. Exp. Med., 1991, 173, 961-969]. Hemoglobin degradation is mediated by serine proteases and aspartic proteases. Aspartic proteases have been shown to be indispensable to parasite growth. A non-selective inhibitor of aspartic proteases, Pepstatin, inhibits the growth of P. falciparum in red blood cells in vitro. The same results have been obtained with analogs of pepstatin [Francis, S. E., Gluzman, I. Y., Oksman, A., Knickerbocker, A., Mueller, R., Bryant, M. L., Sherman, D. R., Russell, D. G., Goldberg, D. E., Molecular characterization and inhibition of a Plasmodium falciparum aspartic hemoglobinase, Embo. J., 1994, 13, 306-317; Moon, R. P., Tyas, L., Certa, U., Rupp, K., Bur, D., Jaquet, H., Matile, H., Loetscher, H., Grueninger-Leitch, F., Kay, J., Dunn, B. M., Berry, C., Ridley, R. G., Expression and characterization of plasmepsin I from Plasmodium falciparum, Eur. J. Biochem., 1997, 244, 552-560]. These results show that inhibition of parasite aspartic proteases interferes with the life cycle of P. falciparum. Consequently, aspartic proteases are targets for antimalarial drug development.

Today no plasmepsin II inhibitor has entered human clinical trials or is in advanced stage of clinical development. The scientific literature reports a certain number of peptidomimetic or substrate-derived plasmepsin II inhibitors [Ersmark, K. et al, J. Med. Chem., 2004, 47, 110-122; Johannsson, P-O. et al, J. Med. Chem., 2004, 47, 3353-3366; Hallberg, A., Samuelsson, B. et al., J. Med. Chem., 2003, 46, 734-746; ibid, Bioorg. Med. Chem., 2003, 11, 1235-1246; ibid, Bioorg. Med. Chem., 2003, 11, 827-841; Nöteberg, D., Larhed, M. et al., J. Comb. Chem., 2003, 5, 456-464; Nezami, A., Freire, E. et al., Biochemistry, 2002, 41, 2273-2280; Haque, T. S., Ellman, J. A. et al., J. Med. Chem., 1999, 42, 1428-1440; Brinner, K. M., Ellamn, J. A. et al., Bioorg. Med. Chem., 2002, 10, 3649-3661; Dolle R. E. et al.; Bioorg. Med. Chem. Lett., 1998, 8, 2315-2320; Dolle R. E. et al., Bioorg. Med. Chem. Lett., 1998, 8, 3203-3206; U.S. Pat. No. 5,734,054 (Pharmacopeia Inc., Dolle R. E. et al.)] which according to the reported data show reasonable inhibitory activity towards the isolated enzyme, but very often fail to conserve this activity in cell based assays or in animal models of malaria. It is of general knowledge that peptidomimetic drugs are potentially metabolically of limited stability and very often might exhibit unfavourable ADME properties preventing them from being active in in vivo situations.

There are some reports of non-peptidic or non-peptidomimetic plasmepsin II inhibitors in the scientific literature [Carcache, D. A., Diederich F. et al., Chem Bio Chem, 2002, 11, 1137-1141; Carcache, D. A., Diederich, F. et al., Helv. Chim. Acta, 2003, 86, 2173-2191; Carcache, D. A., Diederich, F. et al., Helv. Chim. Acta, 2003, 86, 2192-2209]. But these compounds show a rather low activity in the isolated enzyme assay and are therefore not suitable as drugs.

Another class of non-peptidic and non-substrate derived inhibitors of plasmepsin II are disclosed in WO 02/38534 (Actelion Pharmaceuticals Ltd; Boss C. et al.).

Another group of non-peptidomimetic, low-molecular weight plasmepsin II inhibitors is described in WO 02/24649 (Actelion Pharmaceuticals Ltd, Boss, C. et al.), in C. Boss et al., Curr. Med. Chem., 2003, 10, 883-907 and in R. Mueller, M. Huerzeler and C. Boss, Molecules, 2003, 8, 556-564. Although highly active on the isolated enzyme, these molecules suffer from substantial drawbacks with respect to their physicochemical properties such as lipophilicity and solubility in aqueous solutions or under physiological conditions which prevents them from transforming their substantial in vitro activity into physiological situations.

With respect to inhibitory activity towards the plasmodium falciparum enzymes plamsepsin II and plasmepsin IV, the compounds of the present invention are clearly superior to the compounds described in the prior art. This fact manifestates e.g. in the results obtained from cellular assays with compounds contained in the present application as compared to compounds described in prior art documents.

Most importantly compounds of the present invention are inhibitors of not only plasmepsin II but also plasmepsin IV.

The compounds of formula I can be tested according to the assay described below in the experimental part against plasmepsin II, plasmepsin I, plasmepsin IV, human cathepsin D, and human cathepsin E in order to determine their biological activity and their selectivity profile.

### DESCRIPTION OF THE INVENTION

The present invention relates to low molecular weight organic compounds, in particular to substituted 4-aminopiperidines of the formula I: 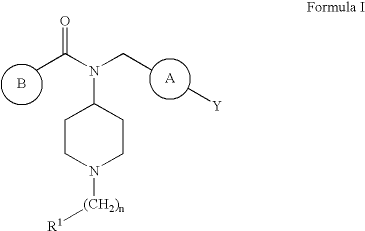
wherein
R¹ represents hydrogen; alkyl, preferably 2-methyl-propyl; alkenyl; alkynyl; cyclopropyl; cyclopentyl; cyclohexyl; cyclohexenyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, and bromine; thienyl that can be mono-substituted with methyl or chlorine; benzothienyl; benzofuranyl; quinolinyl; isoquinolinyl; benzo[1,3]dioxol-5-yl; methoxy-benzo[1,3]dioxol-5-yl; chloro-benzo[1,3]dioxol-5-yl; 2,2-diphenyl-ethyl; 2-phenyl-propyl; 1-[2,6,6-trimethyl-cyclohex-1-enyl]-methyl; pyrrolyl; thiazolyl; or imidazolyl;

n represents the integer 1, 2, or 3; 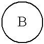
represents 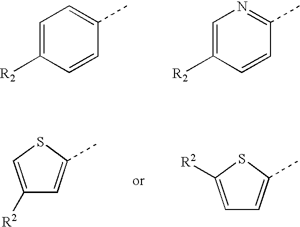
R² represents butyl, pentyl or hexyl; or

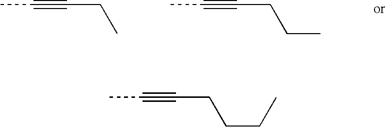

In case 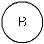
represents

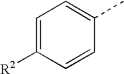

Y represents

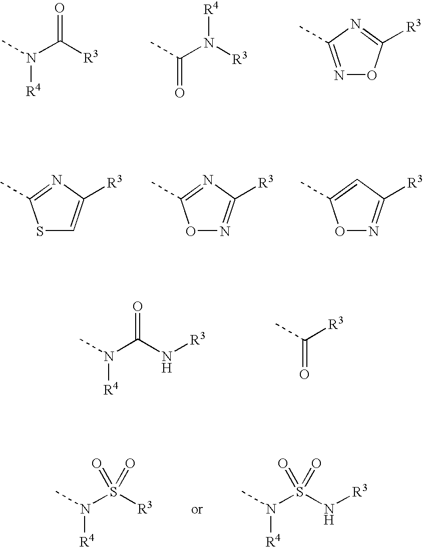

In case 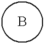
represents 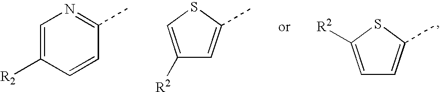
or in case 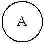
represents 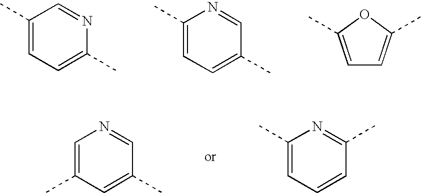
Y represents 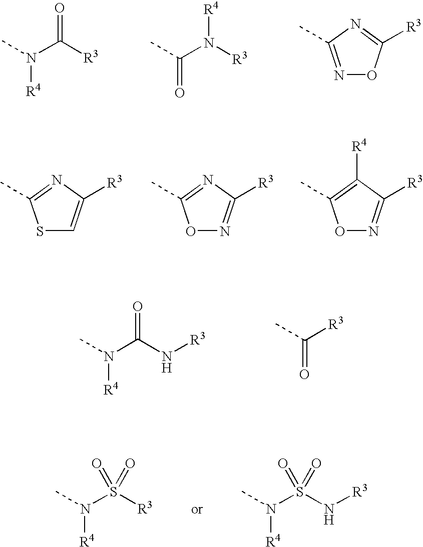
or phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, carboxyl, or the following radicals: 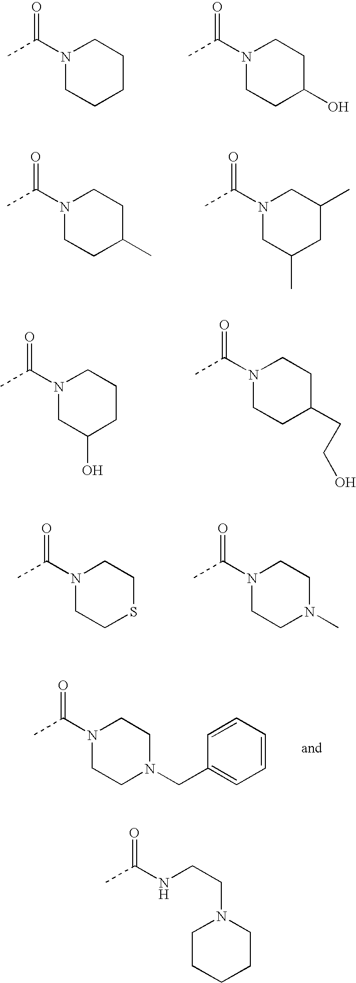
or in case 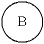
represents 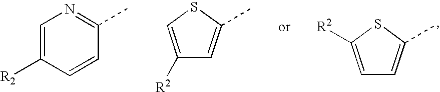
or in case 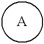
represents 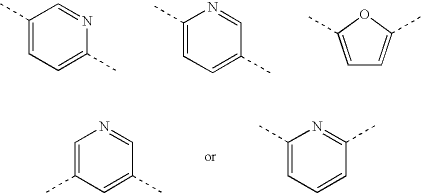
Y can also represent pyridyl that can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, and bromine; thienyl that can be mono-substituted with methyl or chlorine; benzothienyl; benzofuranyl; quinolinyl; isoquinolinyl; benzo[1,3]dioxolyl; 2,2-diphenyl-ethyl; 2-phenyl-propyl; 1-[2,6,6-trimethyl-cyclohex-1-enyl]-methyl; 3-methyl-butyl; phenoxy, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl-oxy, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; or the following radical: 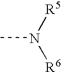
R³ represents alkyl; cycloalkyl; —CF₃; CF₃-alkyl-; alkoxy-alkyl; alkoxy-carbonyl; carboxyl; benzo[1,3]dioxol-5-yl; methoxy-benzo[1,3]dioxol-5-yl; chloro-benzo[1,3]dioxol-5-yl; benzo[1,3]dioxol-5-yl-alkyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenyl-alkyl, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenoxy-methyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; pyridyl-alkyl, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, —CF₃ and halogen; thienyl that can be mono- or di-substituted, wherein the substituents are independently selected from methyl and halogen; thienyl-alkyl; pyrazolyl that can be mono- or di-substituted, wherein the substituents are independently selected from methyl and halogen; benzothienyl; benzofuranyl; benzimidazolyl; benzopyrazolyl; indolyl; indolyl-alkyl; or morpholinyl-alkyl; or in case
Y represents 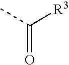
R³ in addition to the above mentioned possibilities may also represent thiomorpholinyl; piperidinyl that can be mono- or di-substituted, wherein the substituents are independently selected from alkyl, hydroxy-alkyl, and hydroxy; piperidinyl-alkyl; morpholinyl; or 1-piperazinyl which can be substituted at the nitrogen atom at position 4 with alkyl, benzyl, pyridyl, or phenyl, wherein the phenyl group can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl;
R⁴ represents hydrogen, methyl, ethyl, isopropyl, or cyclopropyl;
R⁵ and R⁶ represent hydrogen; alkyl; cycloalkyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenyl-alkyl, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyrrolidinyl-alkyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; pyridyl-alkyl, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; piperidinyl-alkyl; morpholinyl-alkyl; 4-methyl-piperazinyl-alkyl; 4-benzyl-piperazinyl-alkyl; alkoxy-alkyl or bis-alkyl-amino-alkyl and may be the same or different; or R⁵ and R⁶ can together form a morpholinyl ring; a thiomorpholinyl ring; a piperidinyl ring which can be mono- or di-substituted, wherein the substituents are independently selected from methyl and hydroxy; or 1-piperazinyl which can be substituted at the nitrogen atom at position 4 with alkyl, benzyl, pyridyl, or phenyl, wherein the phenyl group can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl;
and 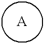
represents 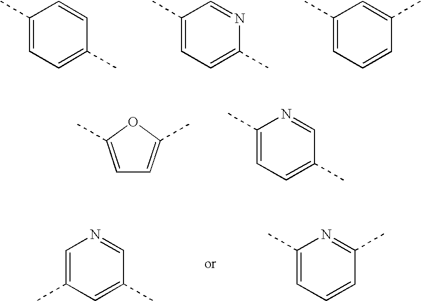
and optically pure enantiomers, mixtures of enantiomers, racemates, diastereomers, mixtures of diastereomers, diastereomeric racemates, mixtures of diastereomeric racemates and meso-forms, as well as salts and solvent complexes of such compounds, and morphological forms.

These substituted 4-aminopiperidines are novel and exhibit useful pharmacodynamic properties.

Objects of the present invention are the 4-aminopiperidines of the formula I above, their optically pure enantiomers, mixtures of enantiomers, racemates, diastereomers, mixtures of diastereomers, diastereomeric racemates, mixtures of diastereomeric racemates and meso-forms, as well as salts and solvent complexes of such compounds, and morphological forms, as such and for use as therapeutically active compounds, pharmaceutical compositions containing such compounds and the preparation of such compounds and pharmaceutical compositions as well as the use of such compounds and compositions for the treatment and/or prevention of diseases demanding the inhibition of parasite aspartic proteases.

The general terms used hereinbefore and hereinafter preferably have, within this disclosure, the following meanings, unless otherwise indicated:

Where the plural form is used for compounds, salts, pharmaceutical compositions, diseases and the like, this is intended to mean also a single compound, salt, or the like.

Any reference to a compound of formula I or a subformula thereof is to be understood as referring also to optically pure enantiomers, mixtures of enantiomers, racemates, diastereomers, mixtures of diastereomers, diastereomeric racemates, mixtures of diastereomeric racemates, and meso-forms, as well as salts (especially pharmaceutically acceptable salts) and solvent complexes (including hydrates) of such compounds, and morphological forms, as appropriate and expedient.

The expression alkyl—alone or in combination with other groups—as used in the present specification means straight or branched chain saturated hydrocarbon groups with 1 to 7, preferably 3 to 6, very preferably 1 to 3, carbon atoms, such as methyl, ethyl, n-propyl, iso-propyl, n-butyl, iso-butyl, sec.-butyl, tert.-butyl, n-pentyl, 2-methyl-propyl, 3,3-dimethyl-propyl, n-hexyl, or n-heptyl.

The expression alkenyl means straight or branched chain hydrocarbon groups with 2 to 7, preferably 3 to 6, carbon atoms, which contain at least one carbon-carbon double bond, such as vinyl, allyl, 2-butenyl, or 3-butenyl.

The expression alkynyl means straight or branched chain hydrocarbon groups with 2 to 7, preferably 3 to 6, carbon atoms, which contain a triple bond, such as ethinyl, propynyl, butynyl, pentynyl, or hexynyl.

The expression alkoxy—alone or in combination with other groups—means alkyl ether groups in which alkyl has the meaning given above, such as methoxy, ethoxy, propoxy, iso-propoxy, iso-butoxy, sec.-butoxy, or tert.-butoxy.

The expression cycloalkyl means a saturated cyclic hydrocarbon ring system with 3 to 6 carbon atoms, i.e. cyclopropyl, cyclobutyl, cyclopentyl and cyclohexyl.

The expression halogen means fluorine, chlorine, bromine or iodine, preferably fluorine, chlorine or bromine.

The expression pharmaceutically acceptable salts encompasses for example salts with inorganic acids or organic acids like hydrochloric or hydrobromic acid; sulfuric acid, phosphoric acid, nitric acid, citric acid, formic acid, acetic acid, maleic acid, tartaric acid, methylsulfonic acid, p-toluolsulfonic acid and the like or in case the compound of formula I is acidic in nature with an inorganic base like an alkali or earth alkali base, e.g. sodium hydroxide, potassium hydroxide, calcium hydroxide etc. For other examples of pharmaceutically acceptable salts, reference can be made to “Salt selection for basic drugs”, Int. J. Pharm. (1986), 33, 201-217.

The compounds of the formula I may contain one or more asymmetric carbon atoms and may be prepared in form of optically pure enantiomers, mixtures of enantiomers such as racemates, diastereomers, mixtures of diastereomers, diastereomeric racemates, mixtures of diastereomeric racemates, or meso-forms. The present invention encompasses all these forms. Mixtures can be separated in a manner known per se, e.g. by column chromatography, thin layer chromatography (TLC), high pressure liquid chromatography (HPLC), crystallization etc.

Preferred are compounds of formula I above wherein R¹ represents hydrogen; alkyl; cyclopropyl; cyclopentyl; cyclohexyl; cyclohexenyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, and bromine; or thienyl that can be mono-substituted with methyl or chlorine; and preferably represents hydrogen; methyl; ethyl; propyl; 2-methyl-propyl; 3,3-dimethyl-propyl; cyclopropyl; or imidazolyl; more preferably ethyl; propyl; 2-methyl-propyl or 3,3-dimethyl-propyl; most preferably 2-methyl-propyl. The preferred meaning of n is the integer 1. The preferred meaning of R² is pentyl or hexyl, preferably pentyl. The preferred meaning of R³ is alkyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenyl-alkyl, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; pyridyl-alkyl, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, —CF₃ and halogen; thienyl that can be mono- or di-substituted, wherein the substituents are independently selected from methyl and chlorine; thienyl-alkyl; benzo[1,3]dioxol-5-yl; or benzo[1,3]dioxol-5-yl-alkyl. The preferred meaning of R⁴ is hydrogen or methyl. The preferred meaning of R⁵ is alkyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenyl-alkyl, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; pyridyl-alkyl, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; piperidinyl-alkyl; morpholinyl-alkyl; 4-methyl-piperazinyl-alkyl; 4-benzyl-piperazinyl-alkyl; alkoxy-alkyl or bis-alkyl-amino-alkyl. More preferably R⁵ represents piperidinyl-alkyl, morpholinyl-alkyl, 4-methyl-piperazinyl-alkyl, benzyl, pyridyl-ethyl, phenyl, 2-pyridyl, 3-pyridyl or 4-pyridyl, wherein these phenyl and pyridyl rings may be mono-, di-, or tri-substituted, wherein the substituents are independently selected from methyl, methoxy, fluorine, chlorine and trifluoromethyl. Even more preferred R⁵ represents benzyl, pyridyl-ethyl, 2-pyridyl, 3-pyridyl or 4-pyridyl, wherein these phenyl and pyridyl rings may be mono- or di-substituted, wherein the substituents are independently selected from methyl, methoxy and chlorine. The preferred meaning of R⁶ is hydrogen. Another preferred meaning of R⁵ and R⁶ is that together they form a morpholinyl ring; a thiomorpholinyl ring; a piperidinyl ring which can be mono- or di-substituted, wherein the substituents are independently selected from methyl and hydroxy; or 1-piperazinyl which can be substituted at the nitrogen atom at position 4 with alkyl, benzyl, pyridyl, or phenyl, wherein the phenyl group can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; more preferably R⁵ and R⁶ together form 1-piperazinyl which can be substituted at the nitrogen atom at position 4 with alkyl, benzyl, pyridyl, or phenyl, wherein the phenyl group can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl. 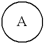
preferably represents 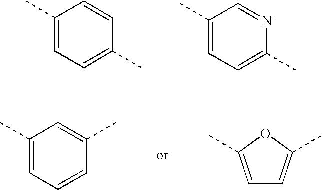
more preferably 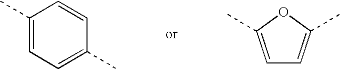 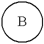
preferably represents 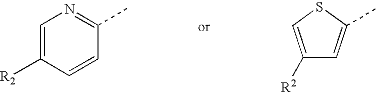
and Y preferably represents 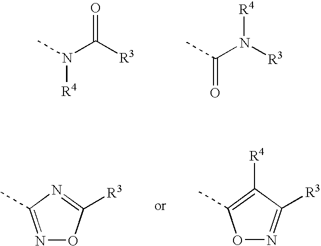
more preferably

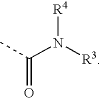

A preferred subgroup of compounds of formula I are compounds wherein 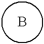
represents 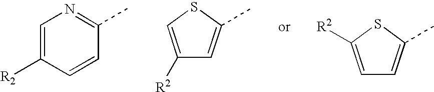
and 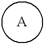
represents 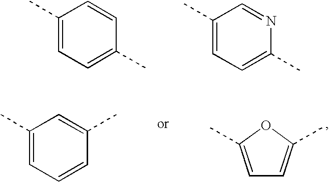
especially

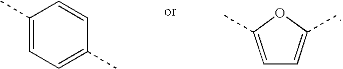

A preferred subgroup of compounds of formula I are those of the formula II 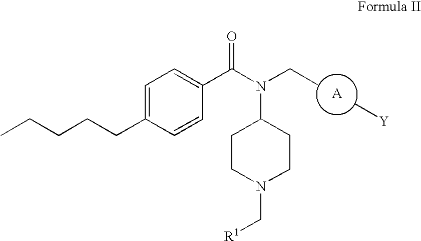
wherein
R¹ and 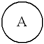
are as defined in formula I above,
Y represents 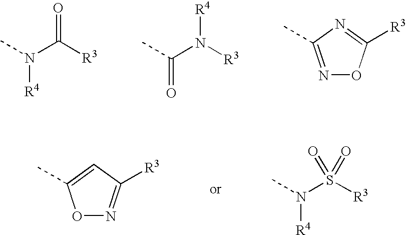
and R³ and R⁴ are as defined in formula I above.

Particular compounds of formula II are those wherein R¹ represents ethyl; propyl; 2-methyl-propyl; 3,3-dimethyl-propyl; cyclopropyl; or imidazolyl; R³ represents alkyl; cyclopropyl; cyclopentyl; cyclohexyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenyl-alkyl, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; pyridyl-alkyl, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, —CF₃ and halogen; thienyl that can be mono- or di-substituted, wherein the substituents are independently selected from methyl and chlorine; thienyl-alkyl; pyrazolyl that can be mono- or di-substituted, wherein the substituents are independently selected from methyl and halogen; benzothienyl; benzofuranyl; benzimidazolyl; benzopyrazolyl; indolyl; indolyl-alkyl; morpholinyl-alkyl; benzo[1,3]dioxol-5-yl; or benzo[1,3]dioxol-5-yl-alkyl; and R⁴ represents hydrogen or methyl.

A preferred group of compounds of formula II are those wherein 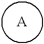
represents 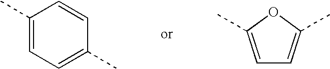
R¹ represents ethyl; propyl; 2-methyl-propyl; 3,3-dimethyl-propyl; cyclopropyl; or imidazolyl; R³ represents alkyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenyl-alkyl, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; pyridyl-alkyl, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, —CF₃ and halogen; thienyl that can be mono- or di-substituted, wherein the substituents are independently selected from methyl and chlorine; thienyl-alkyl; pyrazolyl that can be mono- or di-substituted, wherein the substituents are independently selected from methyl and halogen; benzothienyl; benzofuranyl; benzimidazolyl; benzopyrazolyl; indolyl; indolyl-alkyl; morpholinyl-alkyl; benzo[1,3]dioxol-5-yl; or benzo[1,3]dioxol-5-yl-alkyl; and R⁴ represents hydrogen or methyl.

A further preferred subgroup of compounds of formula I are those of the formula III 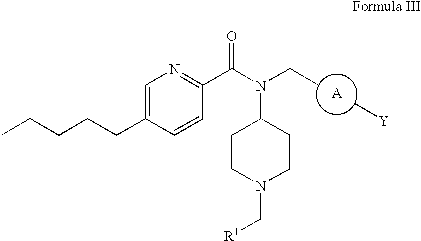
wherein
R¹ and 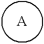
are as defined in formula I above, and
Y represents 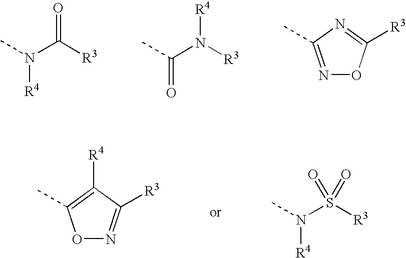
or phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, carboxyl, or the following radicals: 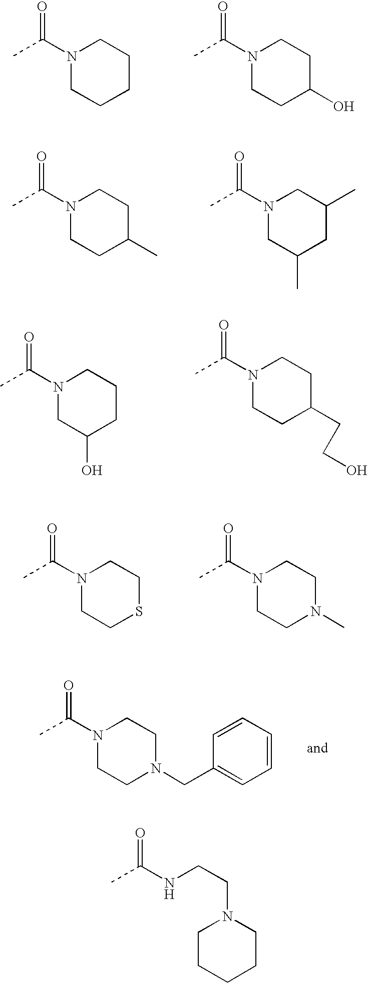
or Y represents pyridyl that can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, and bromine; thienyl that can be mono-substituted with methyl or chlorine; benzothienyl; benzofuranyl; quinolinyl; isoquinolinyl; benzo[1,3]dioxolyl; 2,2-diphenyl-ethyl; 2-phenyl-propyl; 1-[2,6,6-trimethyl-cyclohex-1-enyl]-methyl; 3-methyl-butyl; phenoxy, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl-oxy, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; or the following radical: 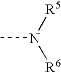
and R³, R⁴, R⁵ and R⁶ are as defined in formula I above.

Particular compounds of formula III are those wherein 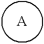
is as defined in formula I above, preferably 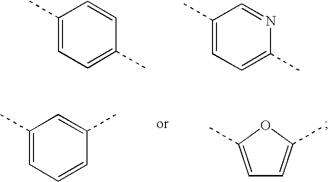
R¹ represents ethyl; propyl; 2-methyl-propyl; 3,3-dimethyl-propyl; cyclopropyl; or imidazolyl; R³ represents alkyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenyl-alkyl, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; pyridyl-alkyl, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, —CF₃ and halogen; thienyl that can be mono- or di-substituted, wherein the substituents are independently selected from methyl and chlorine; thienyl-alkyl; pyrazolyl that can be mono- or di-substituted, wherein the substituents are independently selected from methyl and halogen; benzothienyl; benzofuranyl; benzimidazolyl; benzopyrazolyl; indolyl; indolyl-alkyl; morpholinyl-alkyl; benzo[1,3]dioxol-5-yl; or benzo[1,3]dioxol-5-yl-alkyl; and R⁴ represents hydrogen or methyl.

A group of more preferred compounds of formula III are those wherein 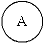
represents 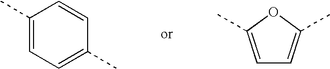
R¹ represents 2-methyl-propyl; 3,3-dimethyl-propyl; or cyclopropyl; R³ represents alkyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenyl-alkyl, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; pyridyl-alkyl, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, —CF₃ and halogen; thienyl that can be mono- or di-substituted, wherein the substituents are independently selected from methyl and chlorine; thienyl-alkyl; pyrazolyl that can be mono- or di-substituted, wherein the substituents are independently selected from methyl and halogen; benzothienyl; benzofuranyl; benzimidazolyl; benzopyrazolyl; indolyl; indolyl-alkyl; morpholinyl-alkyl; benzo[1,3]dioxol-5-yl; or benzo[1,3]dioxol-5-yl-alkyl; and R⁴ represents hydrogen or methyl.

Another preferred subgroup of compounds of formula I are those of the formula IV 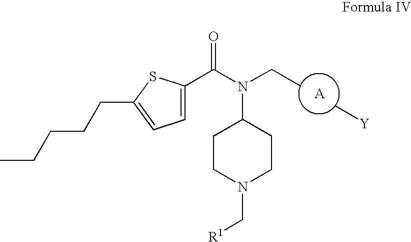
wherein
R¹ and 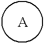
are as defined in formula I above, and
Y represents 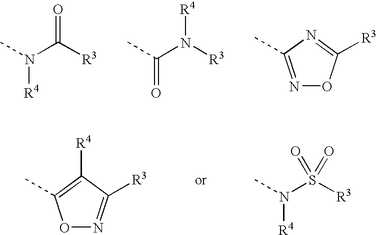
or phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, carboxyl, or the following radicals: 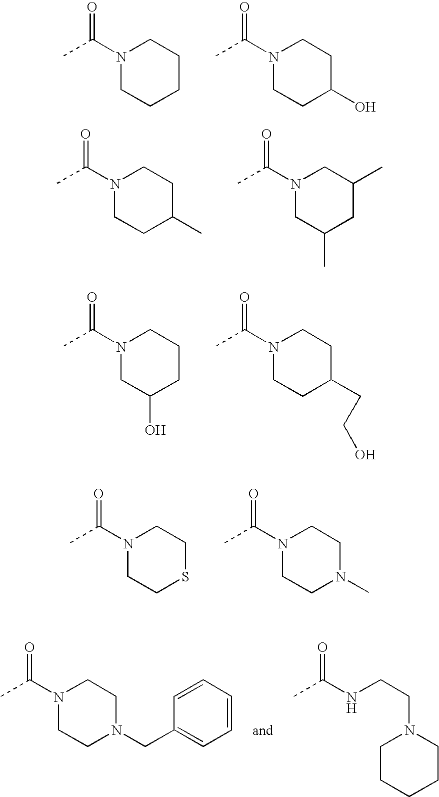
or Y represents pyridyl that can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, and bromine; thienyl that can be mono-substituted with methyl or chlorine; benzothienyl; benzofuranyl; quinolinyl; isoquinolinyl; benzo[1,3]dioxolyl; 2,2-diphenyl-ethyl; 2-phenyl-propyl; 1-[2,6,6-trimethyl-cyclohex-1-enyl]-methyl; 3-methyl-butyl; phenoxy, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl-oxy, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; or the following radical: 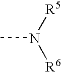
and R³, R⁴, R⁵ and R⁶ are as defined in formula I above.

Particular compounds of formula IV are those wherein R¹ represents ethyl; propyl; 2-methyl-propyl; 3,3-dimethyl-propyl; cyclopropyl; or imidazolyl; R³ represents alkyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenyl-alkyl, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; pyridyl-alkyl, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, —CF₃ and halogen; thienyl that can be mono- or di-substituted, wherein the substituents are independently selected from methyl and chlorine; thienyl-alkyl; pyrazolyl that can be mono- or di-substituted, wherein the substituents are independently selected from methyl and halogen; benzothienyl; benzofuranyl; benzimidazolyl; benzopyrazolyl; indolyl; indolyl-alkyl; morpholinyl-alkyl; benzo[1,3]dioxol-5-yl; or benzo[1,3]dioxol-5-yl-alkyl; and R⁴ represents hydrogen or methyl.

Preferred compounds of formula IV are those wherein 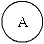
represents 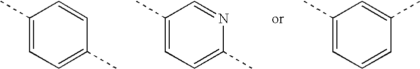
R¹ represents 2-methyl-propyl; 3,3-dimethyl-propyl; cyclopropyl; or imidazolyl; R³ represents alkyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenyl-alkyl, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; pyridyl-alkyl, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, —CF₃ and halogen; thienyl that can be mono- or di-substituted, wherein the substituents are independently selected from methyl and chlorine; thienyl-alkyl; pyrazolyl that can be mono- or di-substituted, wherein the substituents are independently selected from methyl and halogen; benzothienyl; benzofuranyl; benzimidazolyl; benzopyrazolyl; indolyl; indolyl-alkyl; morpholinyl-alkyl; benzo[1,3]dioxol-5-yl; or benzo[1,3]dioxol-5-yl-alkyl; and R⁴ represents hydrogen or methyl.

Still another preferred subgroup of compounds of formula I are those of the formula V 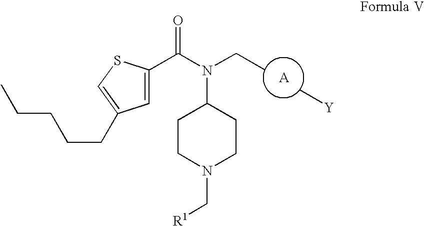
wherein
R¹ and 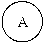
are as defined in formula I above, and
Y represents 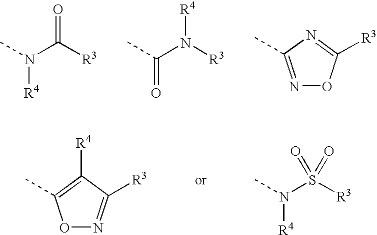
or phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, carboxyl, or the following radicals: 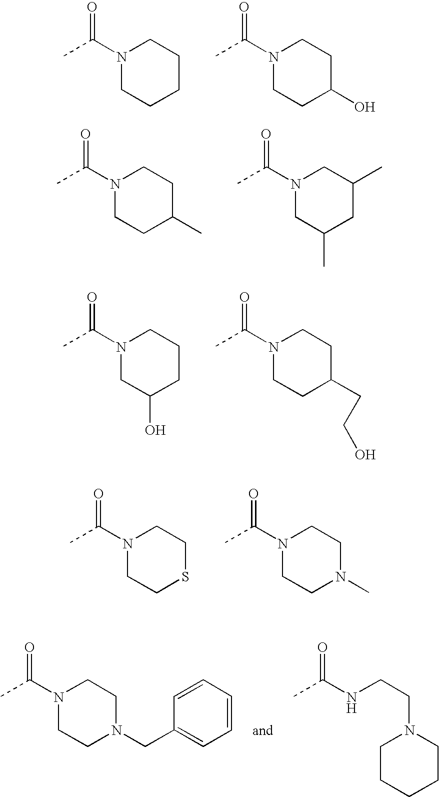
or Y represents pyridyl that can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, and bromine; thienyl that can be mono-substituted with methyl or chlorine; benzothienyl; benzofuranyl; quinolinyl; isoquinolinyl; benzo[1,3]dioxolyl; 2,2-diphenyl-ethyl; 2-phenyl-propyl; 1-[2,6,6-trimethyl-cyclohex-1-enyl]-methyl; 3-methyl-butyl; phenoxy, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl-oxy, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; or the following radical: 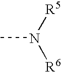
and R³, R⁴, R⁵ and R⁶ are as defined in formula I above.

Preferred compounds of formula V are those wherein 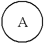
is as defined in formula I above, preferably 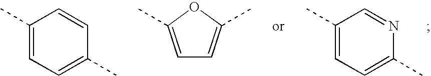
R¹ represents 2-methyl-propyl; 3,3-dimethyl-propyl; cyclopropyl; or imidazolyl; R³ represents alkyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenyl-alkyl, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; pyridyl-alkyl, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, —CF₃ and halogen; thienyl that can be mono- or di-substituted, wherein the substituents are independently selected from methyl and chlorine; thienyl-alkyl; pyrazolyl that can be mono- or di-substituted, wherein the substituents are independently selected from methyl and halogen; benzothienyl; benzofuranyl; benzimidazolyl; benzopyrazolyl; indolyl; indolyl-alkyl; morpholinyl-alkyl; benzo[1,3]dioxol-5-yl; or benzo[1,3]dioxol-5-yl-alkyl; and R⁴ represents hydrogen or methyl.

Another subgroup of preferred compounds of the formula I are those of the formula VI 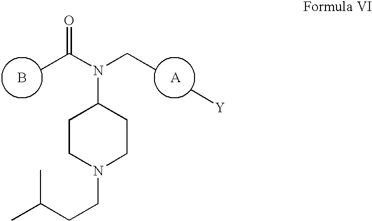
wherein
Y, 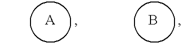
R³, R⁴, R⁵ and R⁶ are as defined in formula I above,
and R² represents pentyl or hexyl, preferably pentyl.

Preferred compounds of formula VI are those wherein 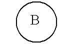
represents

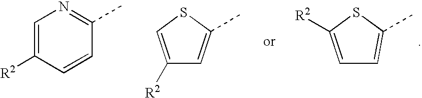

Another group of preferred compounds of formula VI are those wherein 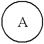
represents 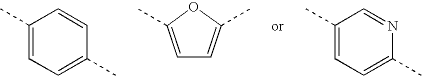 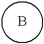
represents 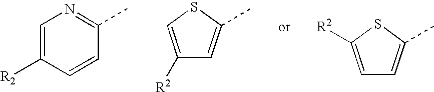
R² represents pentyl; and
Y represents

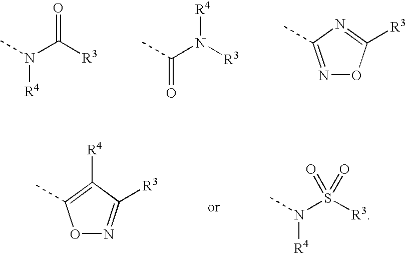

Another group of preferred compounds of formula VI are those wherein 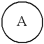
represents 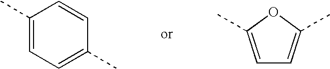 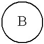
represents 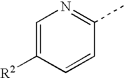
R² represents pentyl; and
Y represents

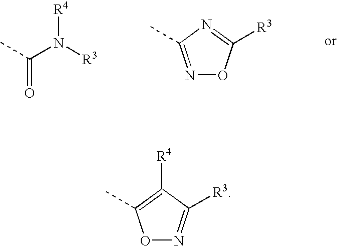

Another group of preferred compounds of formula VI are those wherein 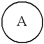
represents 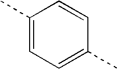 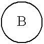
represents 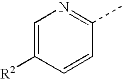
R² represents pentyl; and
Y represents

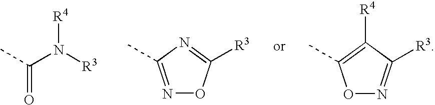

Another group of preferred compounds of formula VI are those wherein 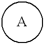
represents 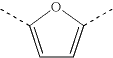 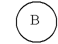
represents 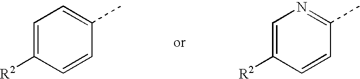
R² represents pentyl; and
Y represents phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, carboxyl, or the following radicals: 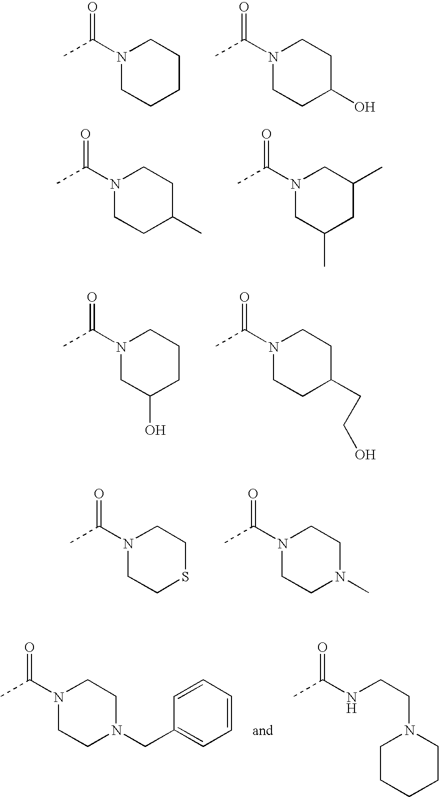
or Y represents pyridyl that can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, and bromine; thienyl that can be mono-substituted with methyl or chlorine; benzothienyl; benzofuranyl; quinolinyl; isoquinolinyl; benzo[1,3]dioxolyl; 2,2-diphenyl-ethyl; 2-phenyl-propyl; 1-[2,6,6-trimethyl-cyclohex-1-enyl]-methyl; 3-methyl-butyl; phenoxy, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; or pyridyl-oxy, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl.

Another group of preferred compounds of formula VI are those wherein 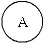
represents 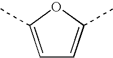 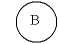
represents 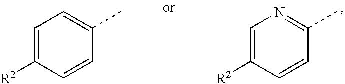
preferably 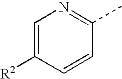
R² represents pentyl; and
Y represents phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, with methyl; thienyl that can be mono-substituted with methyl; benzothienyl; benzofuranyl; quinolinyl; or isoquinolinyl.

Another group of preferred compounds of formula VI are those wherein 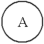
represents 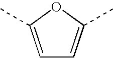 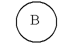
represents 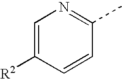
R² represents pentyl; and
Y represents phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; or pyridyl that can be mono-, or di-substituted, wherein the substituents are independently selected from halogen, alkyl, alkoxy, —CF₃, alkoxy-carbonyl, and carboxyl.

Another subgroup of preferred compounds of the formula I are those of the formula VII 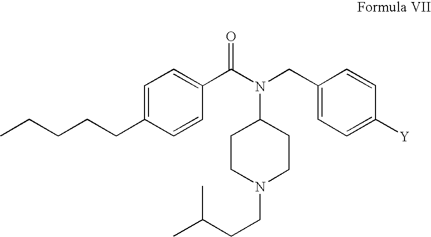
wherein Y represents 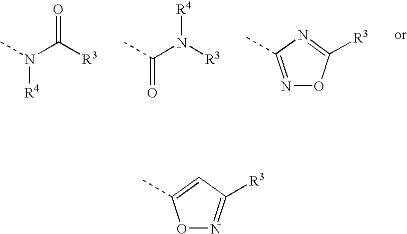
and R³ and R⁴ are as defined in formula I above. A preferred subgroup of compounds of formula VII are compounds wherein Y represents

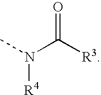

Another particular subgroup of compounds of formula VII are compounds wherein Y represents

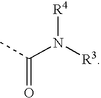

Another particular subgroup of compounds of formula VII are compounds wherein Y represents

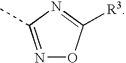

Another preferred subgroup of compounds of formula VII are compounds wherein Y represents 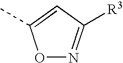
and R³ is as defined in formula I above.

Another subgroup of particularly preferred compounds of the formula I are compounds wherein 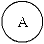
represents 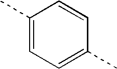 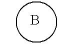
represents 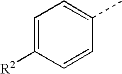
Y represents 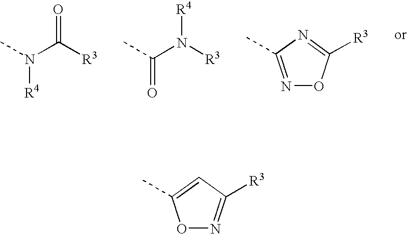
R³ represents alkyl; methoxy-alkyl; trifluoromethyl-alkyl; cyclopropyl; phenyl-alkyl, wherein the phenyl ring can be mono-, or di-substituted, wherein the substituents are independently selected from alkyl, alkoxy, and halogen; phenyl that can be mono-, di-, or tri-substituted, wherein the substituents are independently selected from halogen, alkoxy, —CF₃, —OCF₃, —CN, —COOH, and alkoxy-carbonyl; benzo[1,3]dioxol-5-yl; benzo[1,3]dioxol-5-yl-alkyl; pyridyl that can be mono-, or di-substituted, wherein the substituents are independently selected from alkyl, hydroxy, and alkoxy; pyridyl-alkyl; furanyl that can be mono- or di-substituted, wherein the substituents are independently selected from methyl, halogen, and —CF₃; thienyl that can be mono- or di-substituted, wherein the substituents are independently selected from methyl and halogen; thienyl-alkyl; benzothienyl; benzimidazolyl; indolyl-alkyl; or pyrazolyl that can be mono- or di-substituted, wherein the substituents are independently selected from methyl and halogen; and
R⁴ represents hydrogen or methyl.

Another subgroup of particularly preferred compounds of the formula I are compounds wherein 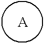
represents 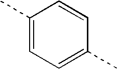 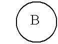
represents 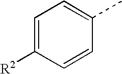
Y represents 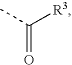
and
R³ represents thiomorpholinyl; piperidinyl that can be mono- or di-substituted, wherein the substituents are independently selected from alkyl, hydroxy-alkyl, and hydroxy; piperidinyl-alkyl; morpholinyl; morpholinyl-alkyl; or 1-piperazinyl which can be substituted at the nitrogen atom at position 4 with alkyl, benzyl, pyridyl, or phenyl, wherein the phenyl group can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl.

Another subgroup of especially preferred compounds of the formula I are compounds wherein

R¹ represents alkyl; cyclopropyl; cyclohexenyl; phenyl that can be mono-substituted with alkoxy or hydroxy; pyridyl; furanyl that can be mono-substituted with hydroxy-methyl; thienyl; pyrrolyl; thiazolyl; or imidazolyl;

n represents the integer 1, 2, or 3; 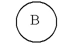
represents 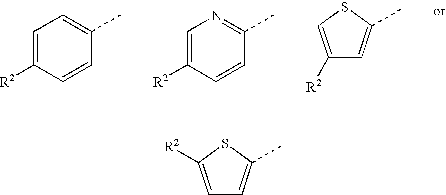
R² represents pentyl or

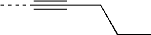

In case 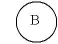
represents 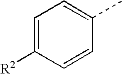
Y represents

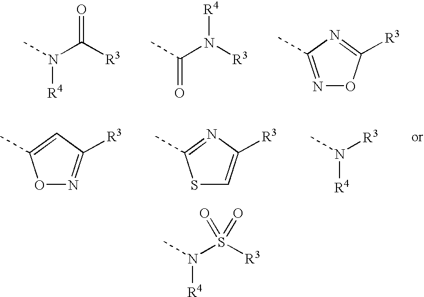

In case 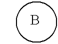
represents 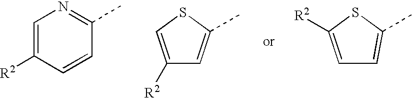
or in case 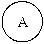
represents 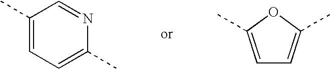
Y represents 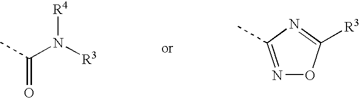
or phenyl that can be mono- or di-substituted, wherein the substituents are independently selected from halogen, alkyl, alkoxy, —CF₃, hydroxy, cyano, carboxyl, or the following radicals: 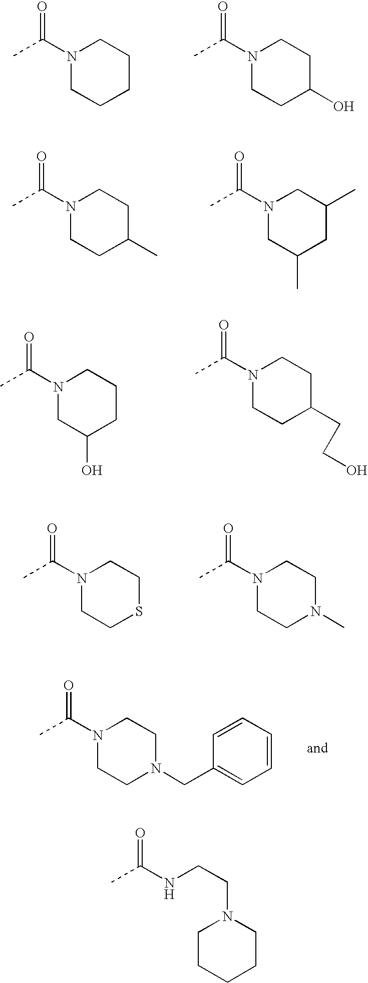
or in case 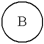
represents 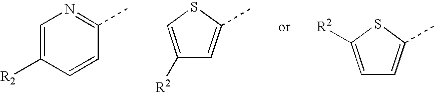
or in case 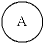
represents 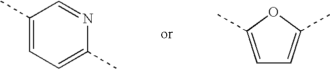
Y can also represent the following radical: 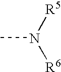
R³ represents alkyl; cycloalkyl; —CF₃; CF₃-alkyl-; alkoxy-alkyl; alkoxy-carbonyl; phenyl that can be mono-, di-, or tri-substituted, wherein the substituents are independently selected from halogen, alkyl, alkoxy, —CF₃, —OCF₃, and cyano; phenyl-alkyl, preferably benzyl or phenyl-ethyl, wherein the phenyl ring can be mono- or di-substituted, wherein the substituents are independently selected from alkyl, halogen, alkoxy, and —CF₃; phenoxy-methyl; pyridyl that can be mono-substituted with alkyl or alkoxy; pyridyl-alkyl, preferably pyridyl-methyl; furanyl that can be di-substituted, wherein the substituents are independently selected from methyl and —CF₃; thienyl that can be mono- or di-substituted, wherein the substituents are independently selected from halogen; thienyl-alkyl; pyrazolyl that is di-substituted, wherein the substituents are independently selected from methyl and halogen; benzothienyl; benzimidazolyl; benzopyrazolyl; indolyl-alkyl; morpholinyl-alkyl; benzo[1,3]dioxol-5-yl; or benzo[1,3]dioxol-5-yl-alkyl;
R⁴ represents hydrogen or methyl;
R⁵ and R⁶ represent alkyl; phenyl-alkyl, preferably benzyl; or pyridyl; or R⁵ and R⁶ together form a morpholinyl ring; a thiomorpholinyl ring; a piperidinyl ring; or 1-piperazinyl which can be substituted at the nitrogen atom at position 4 with alkyl, benzyl, or pyridyl; and 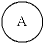
represents

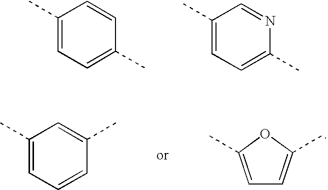

The present invention also relates to compounds of formulae I to VII wherein the meanings of one or more of the substituents and symbols as defined for formulae I to VII, are replaced by their preferred meanings as defined herein, such as those defined for the above-given preferred compounds.

Preferred compounds of the present invention are:

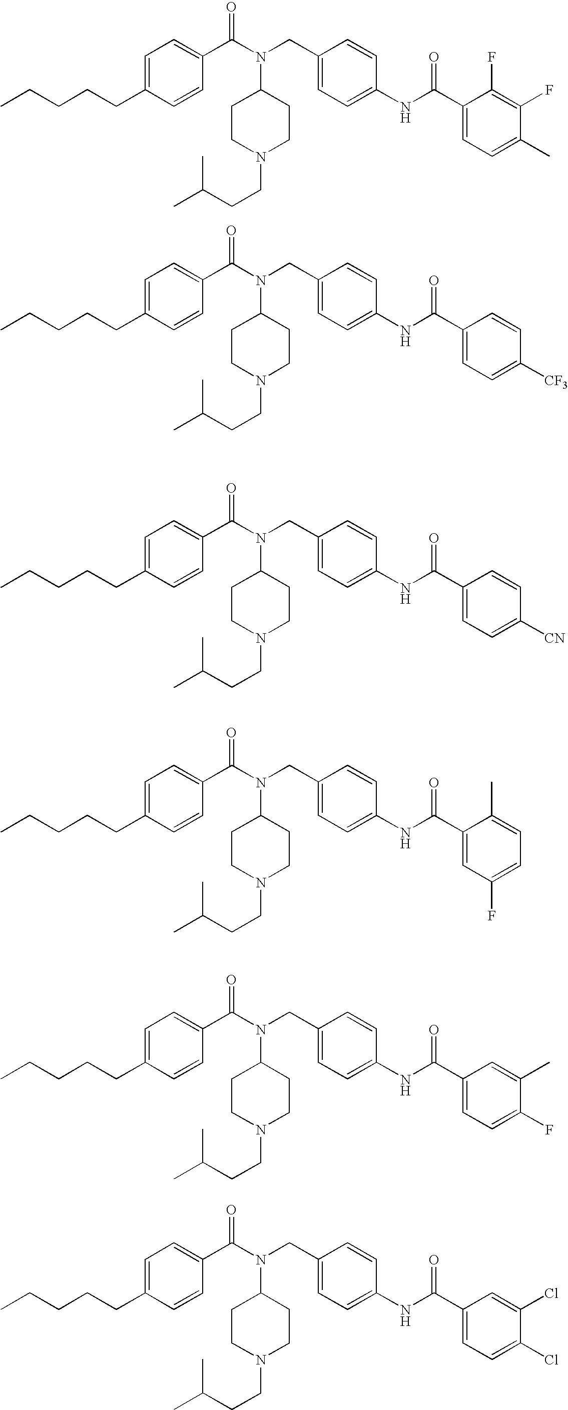

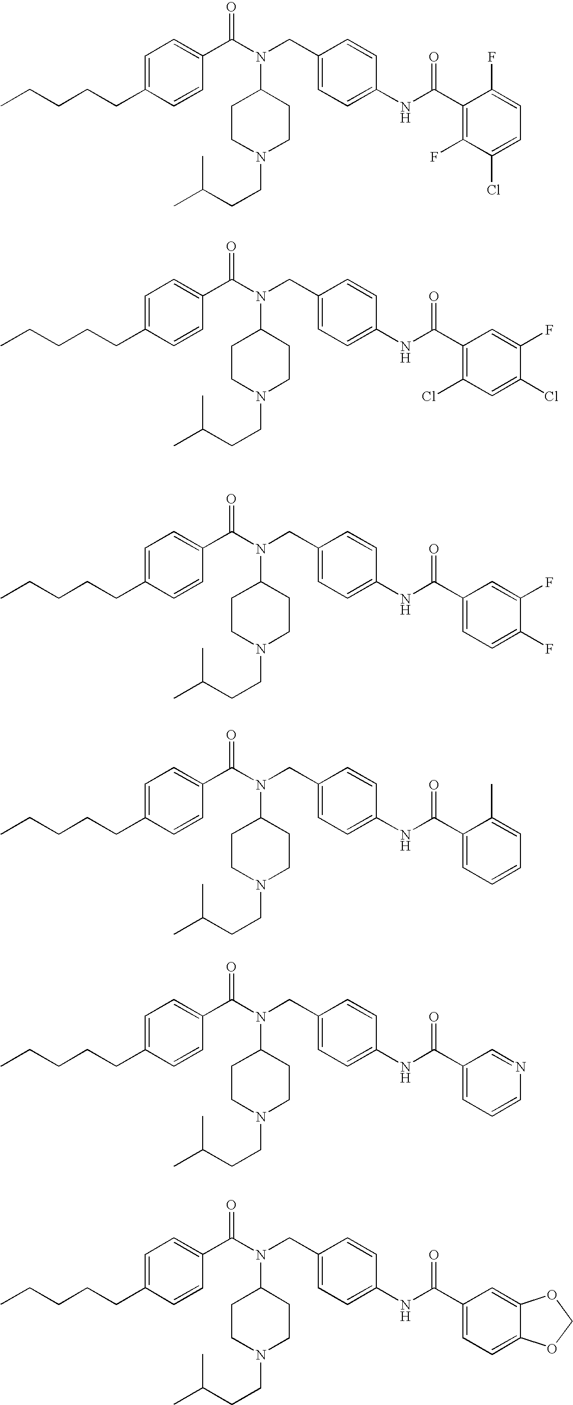

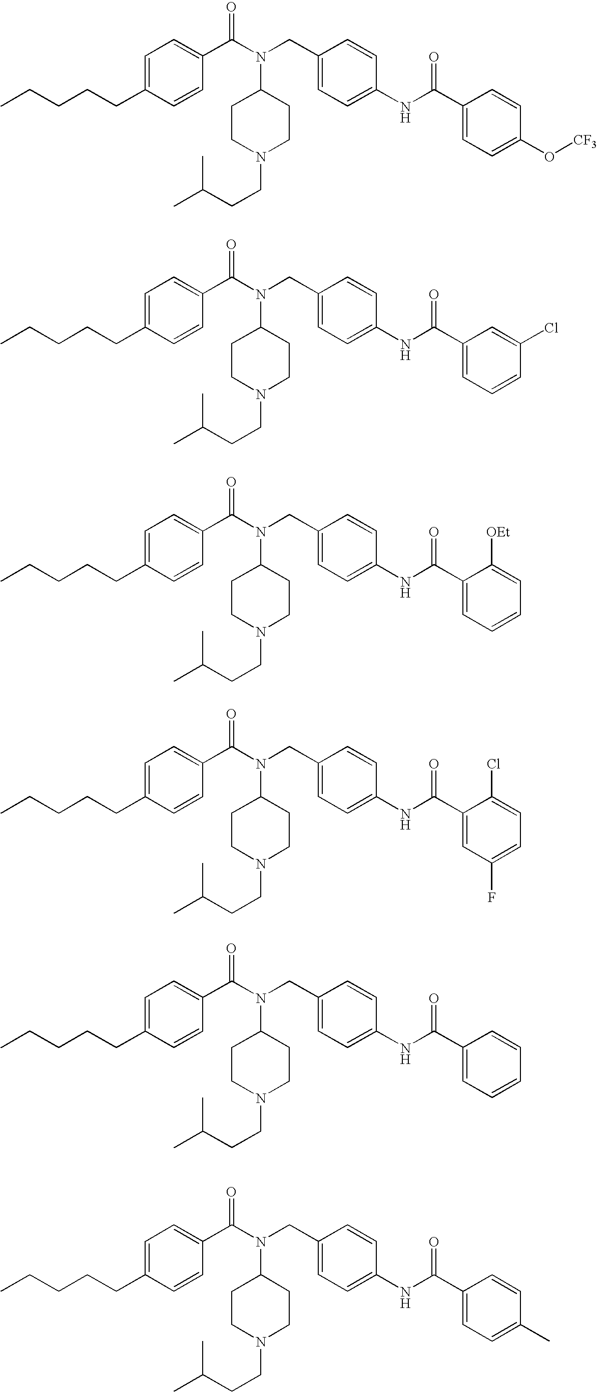

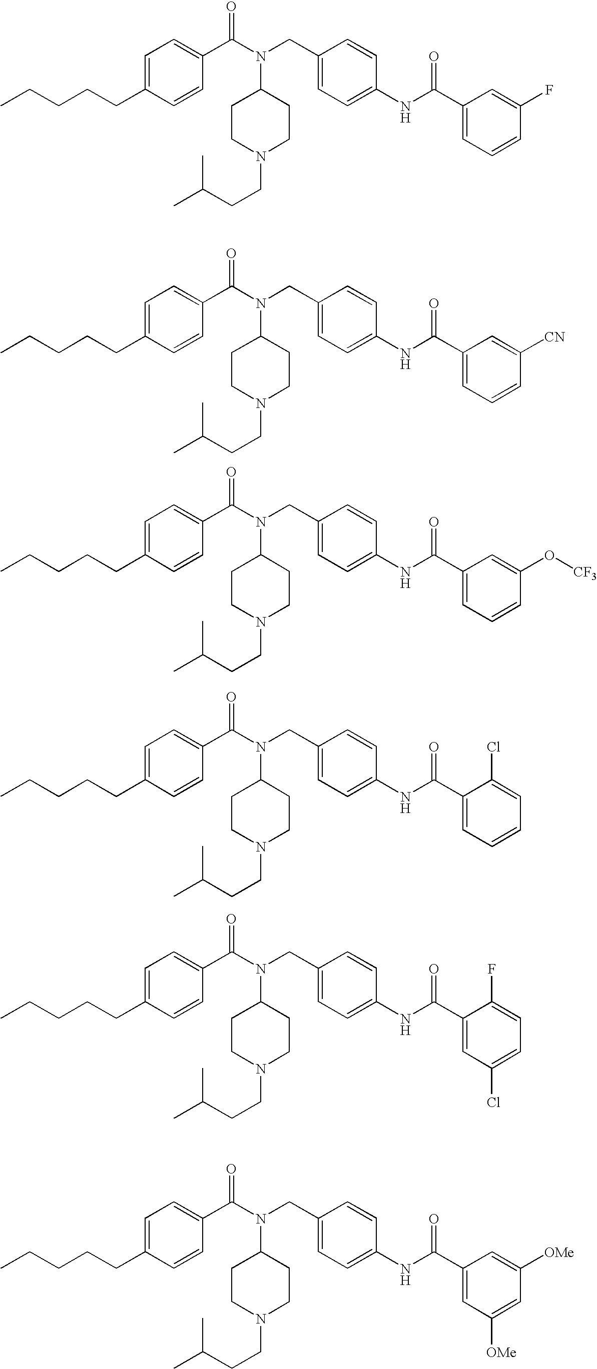

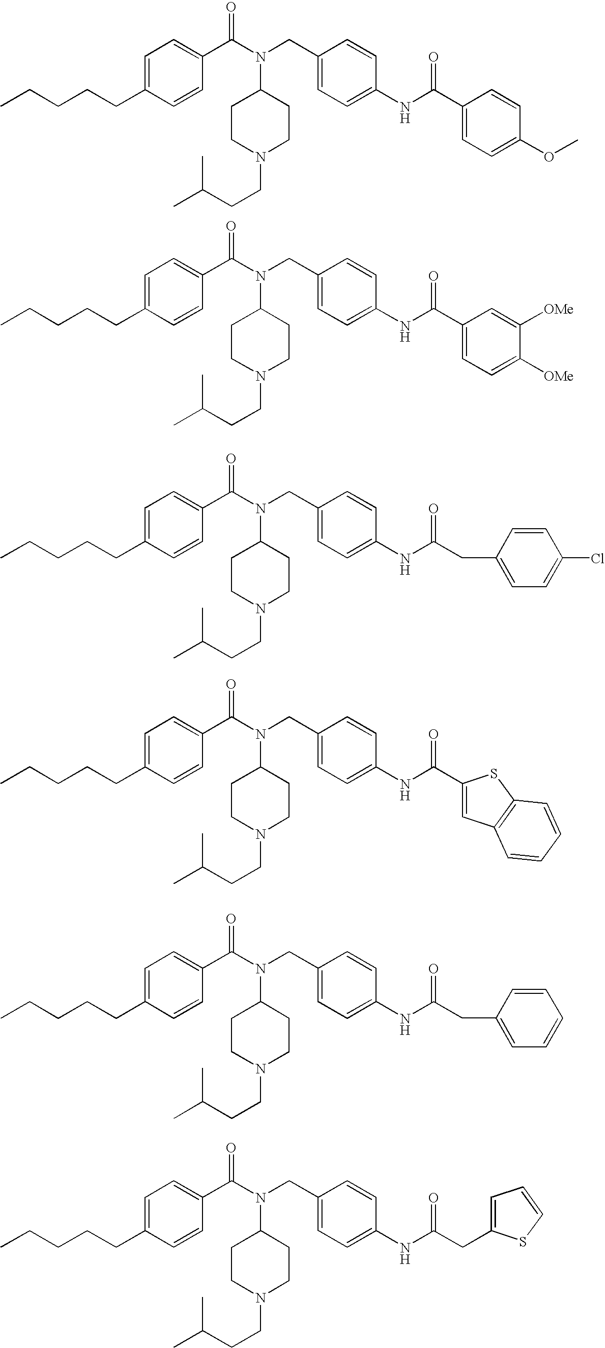

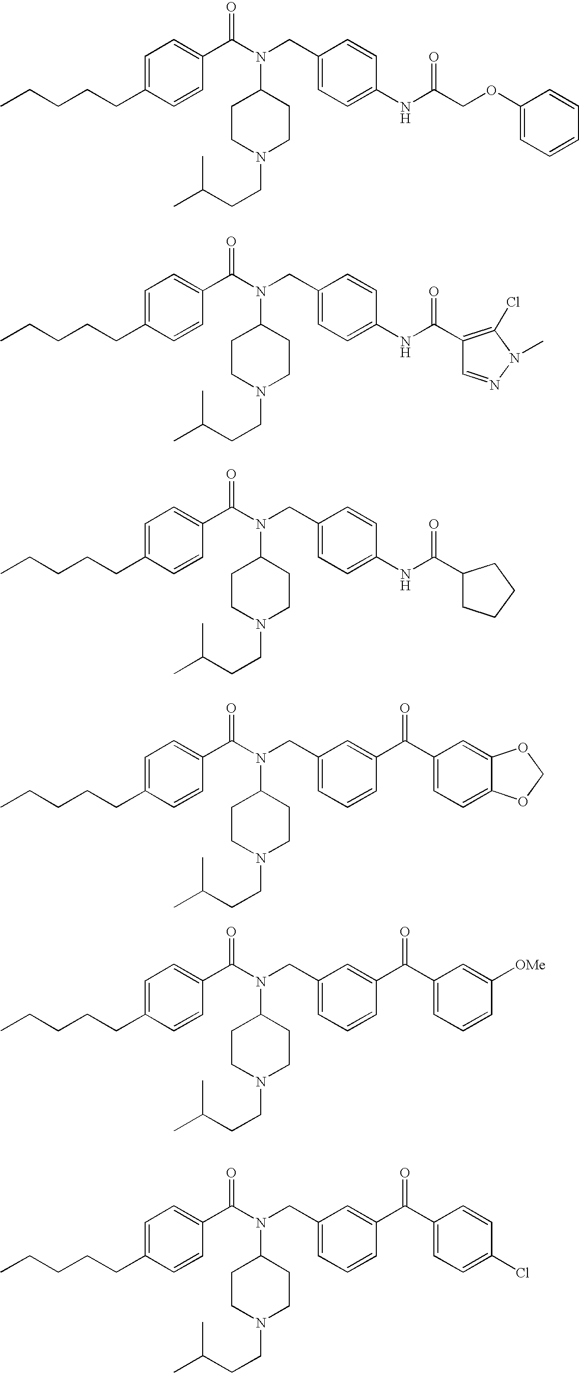

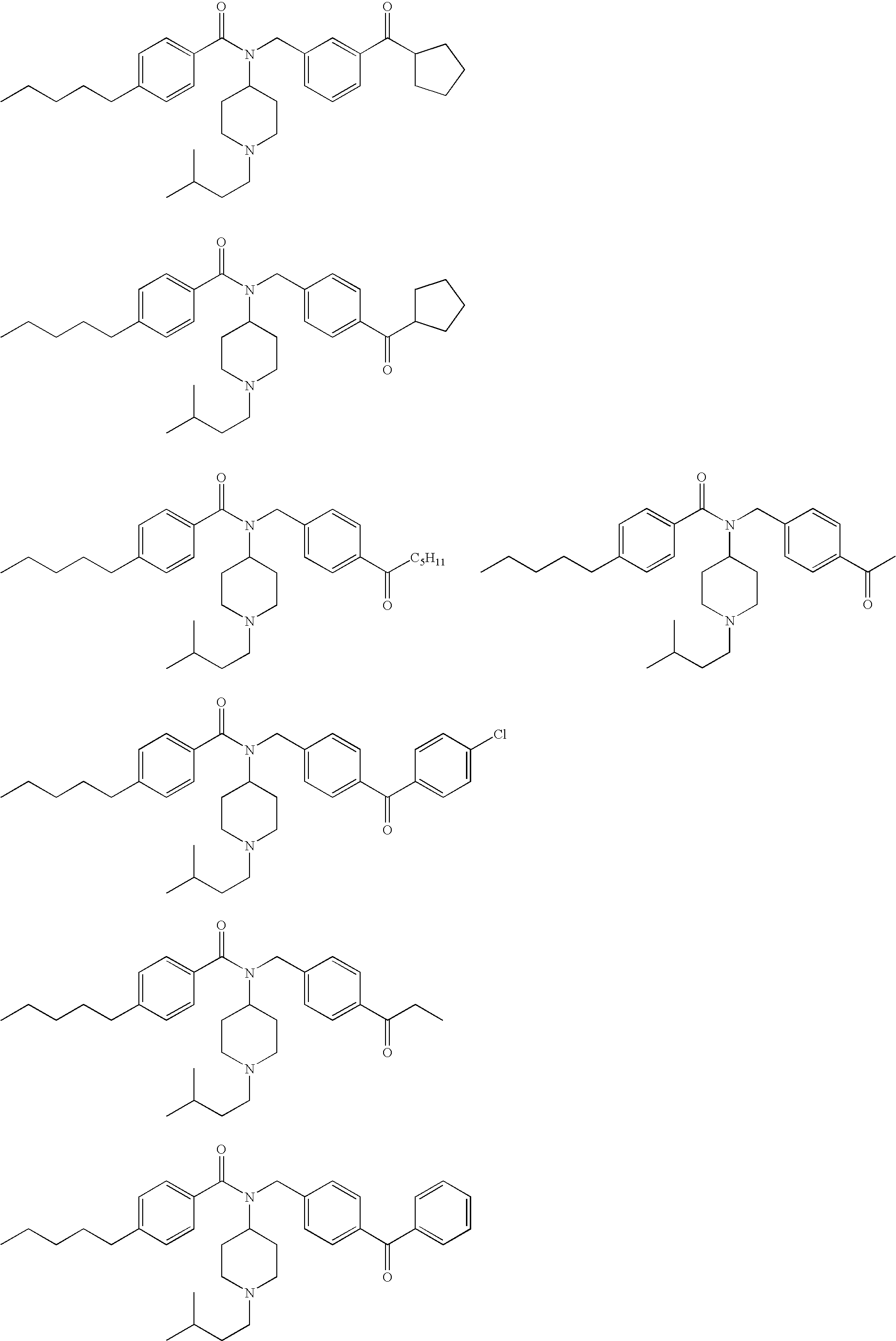

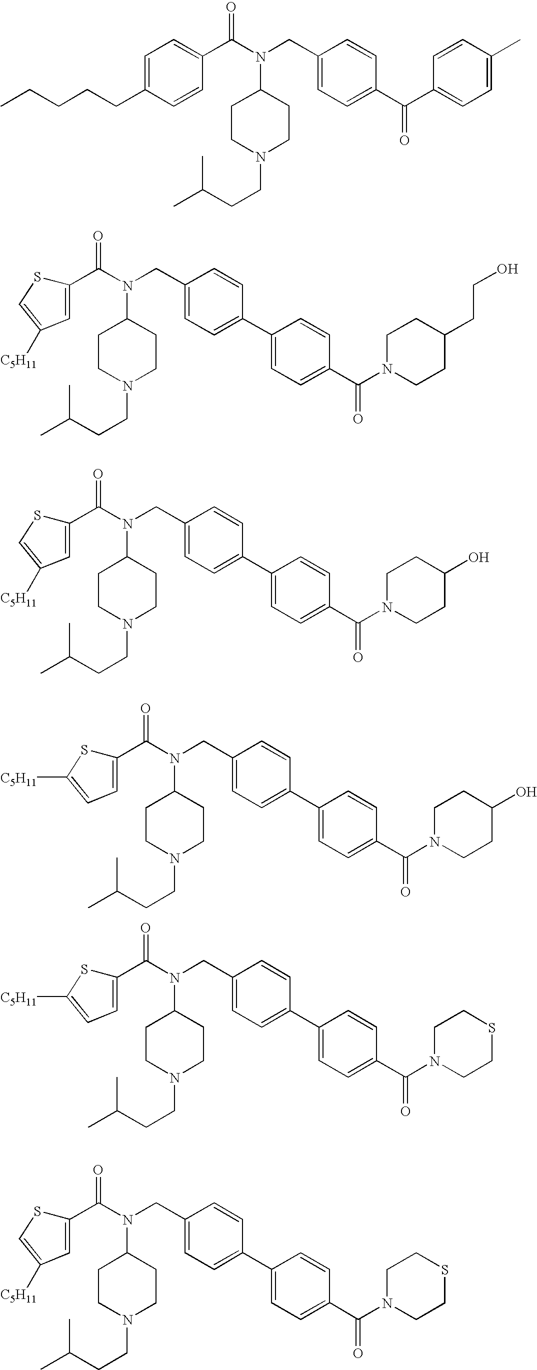

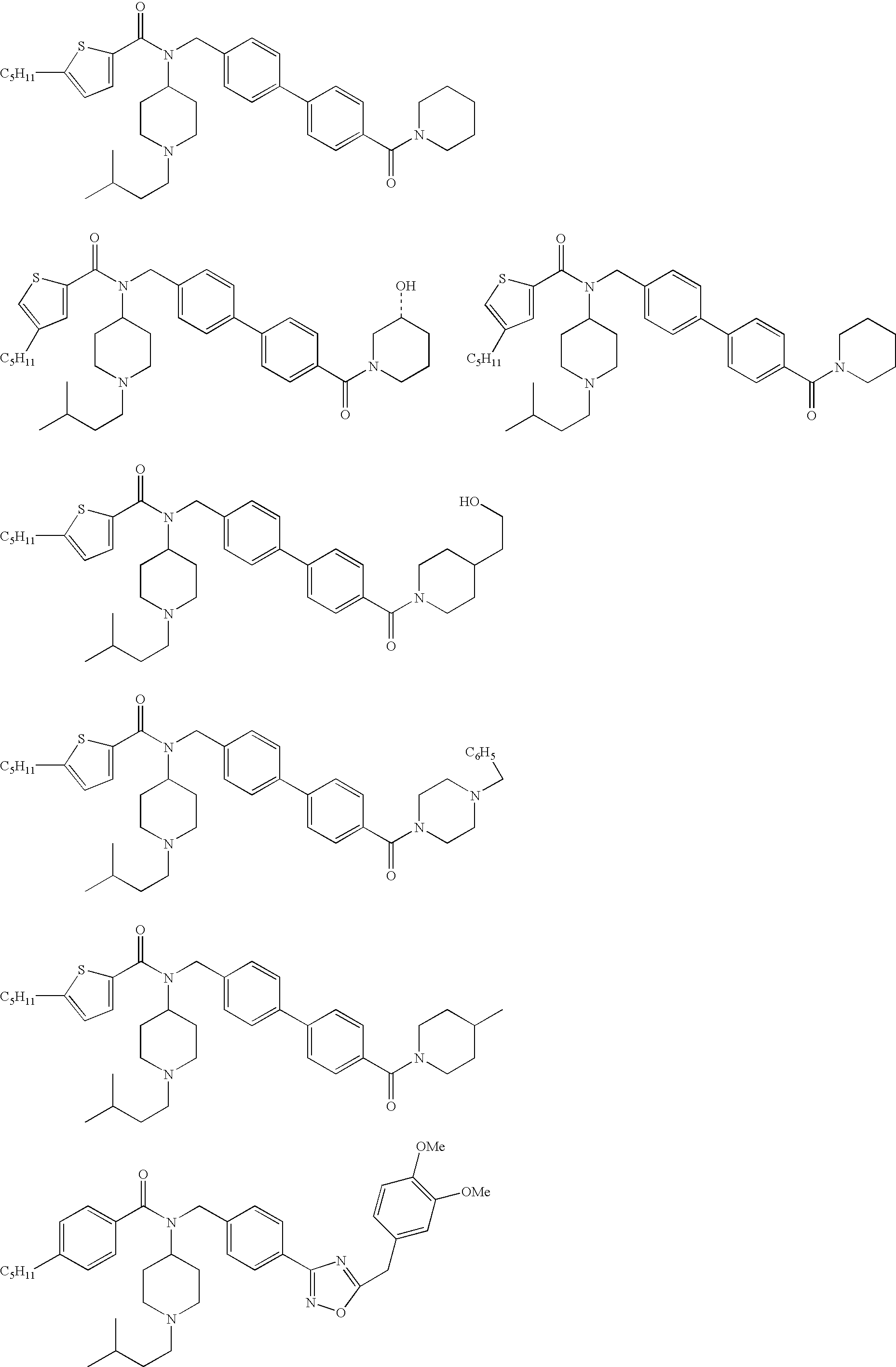

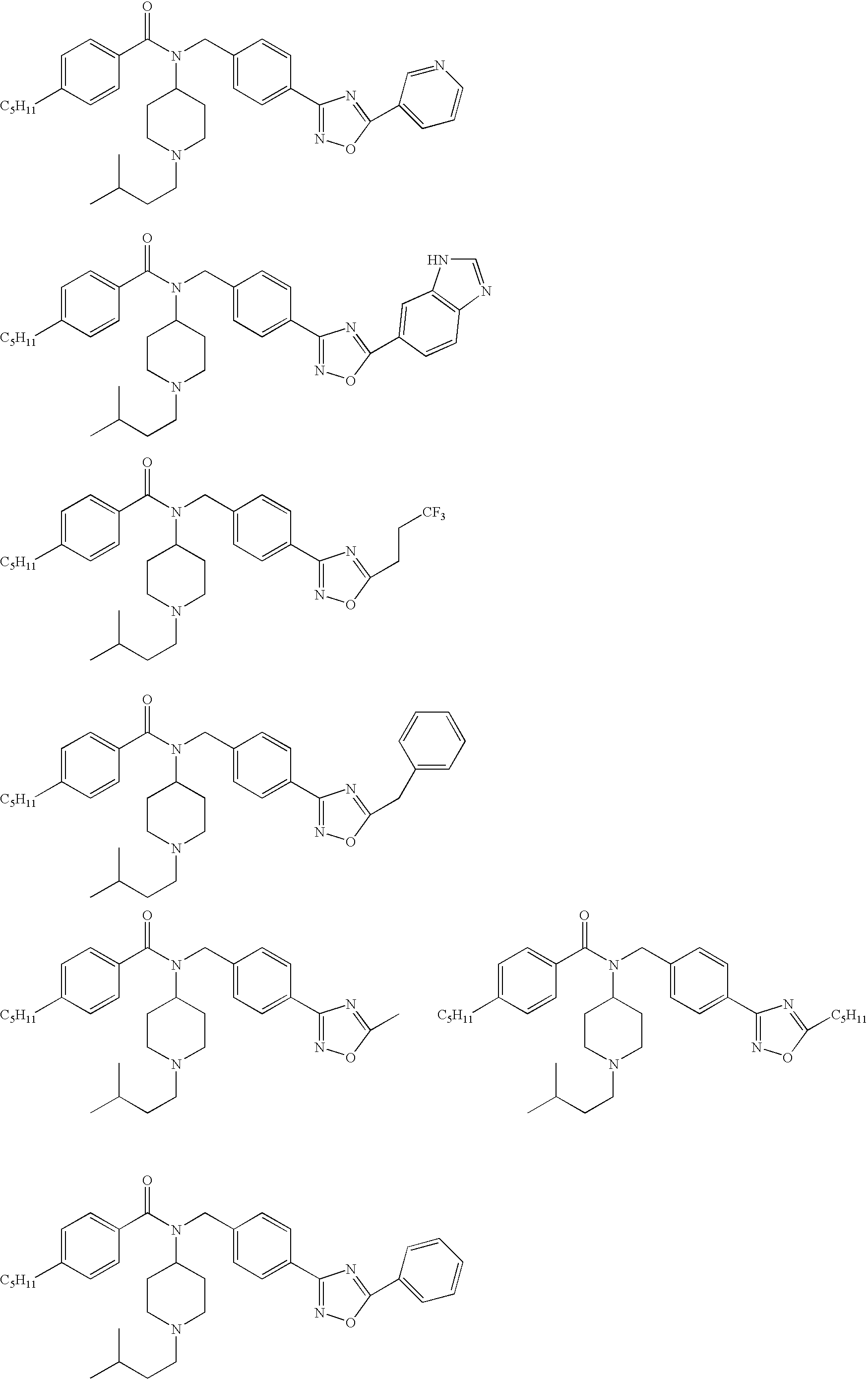

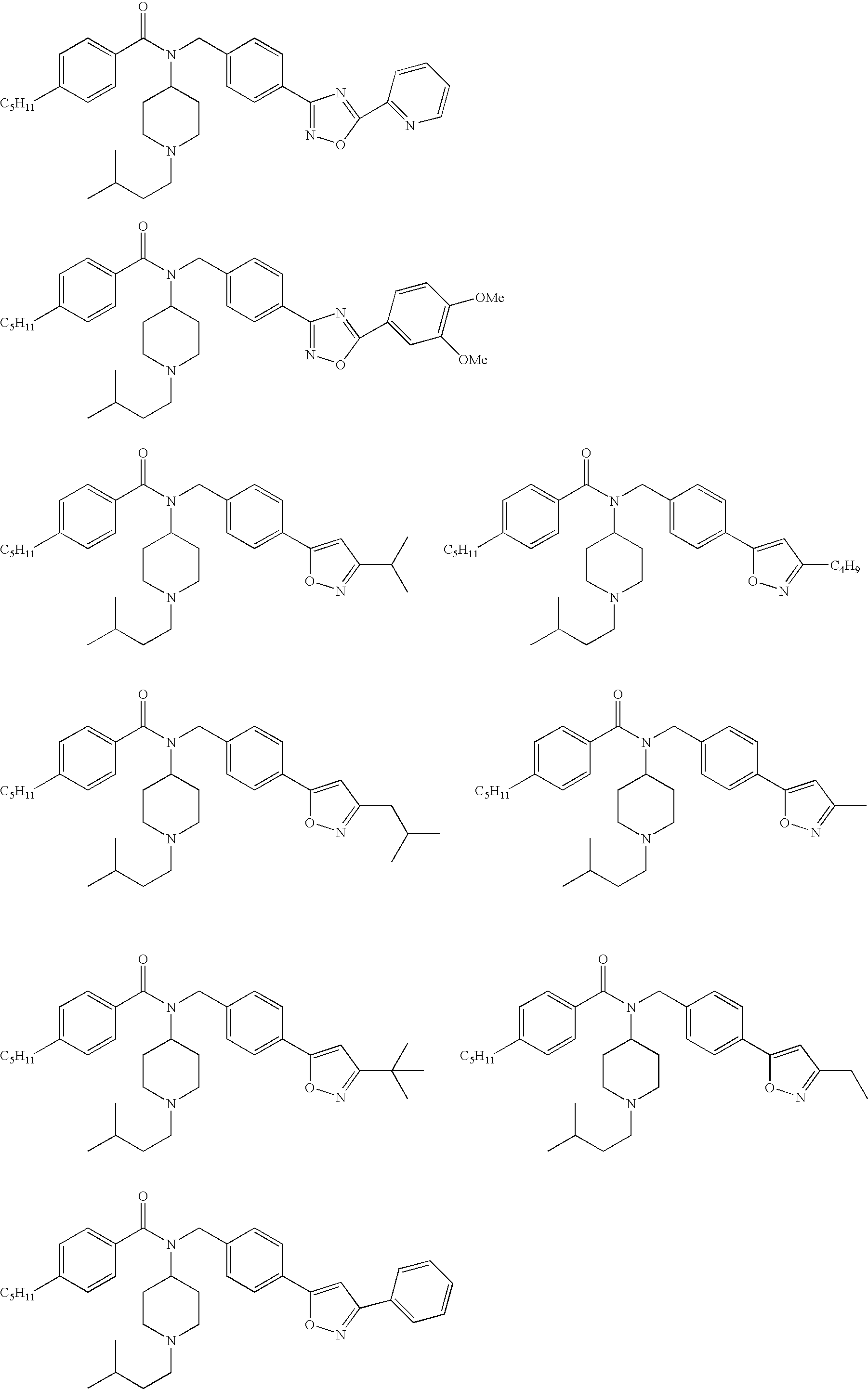

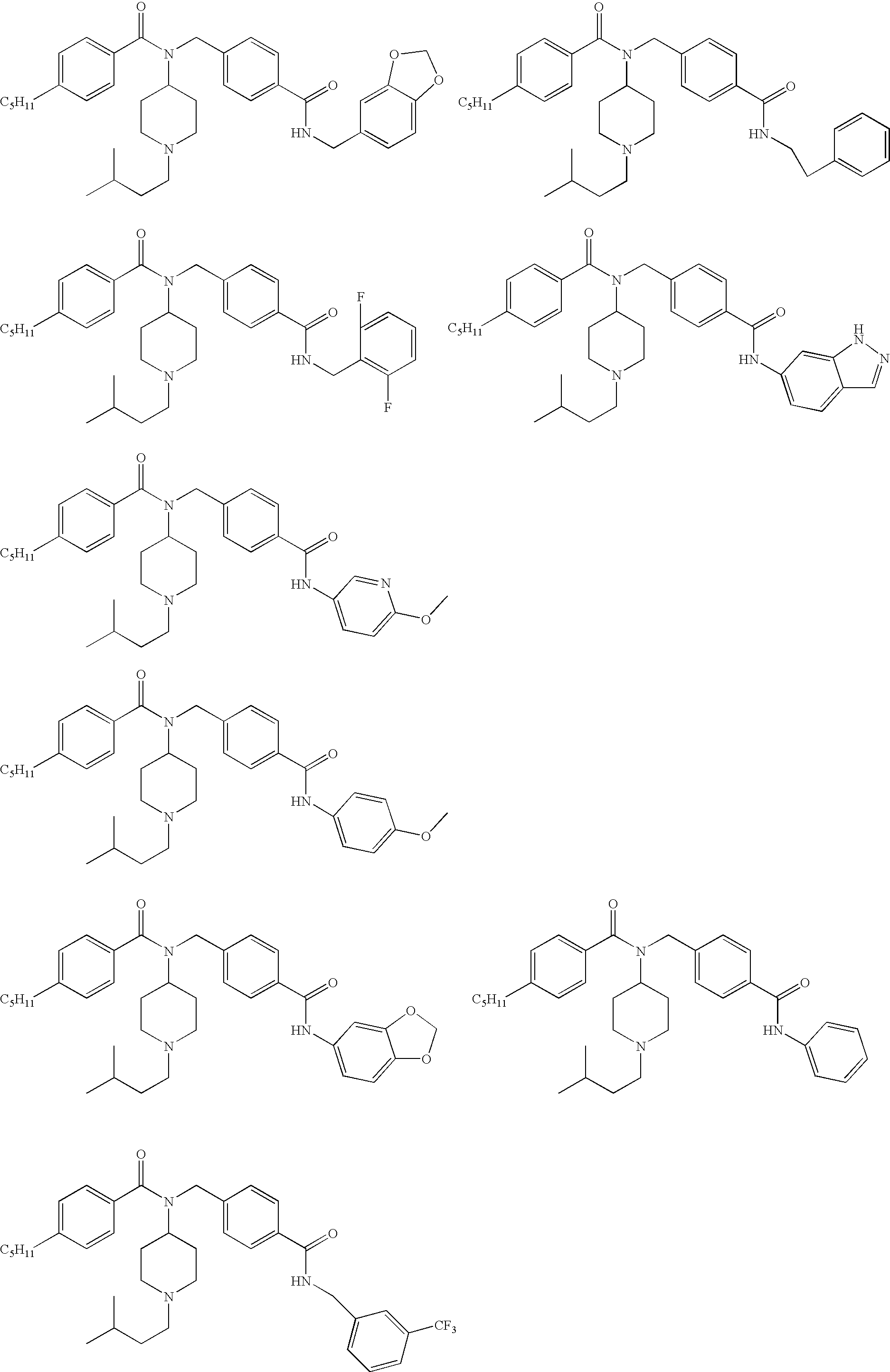

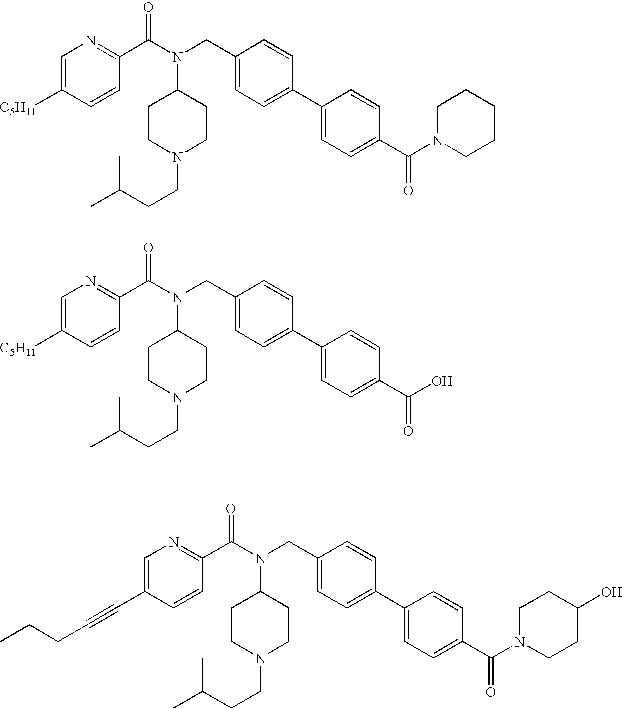

Further very preferred compounds of the present invention are:

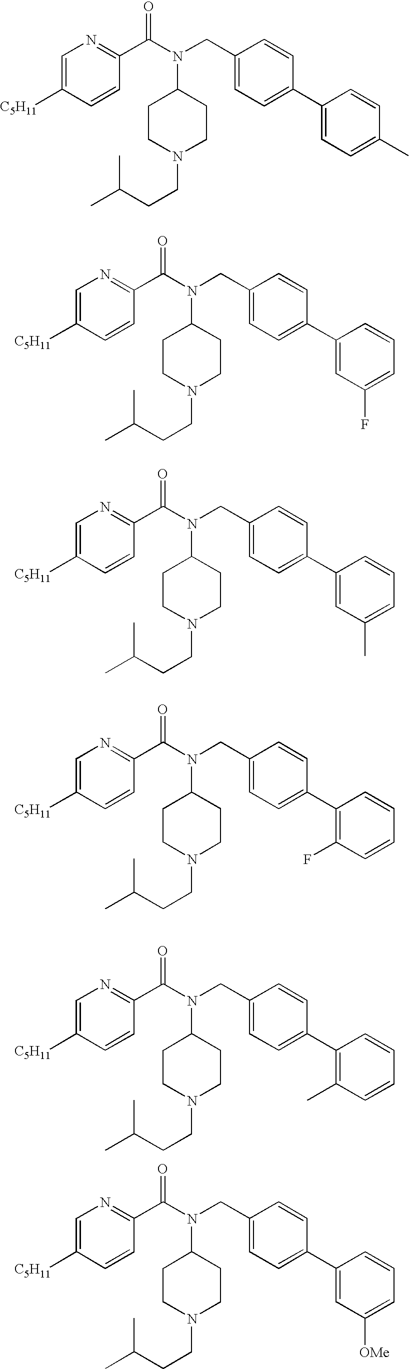

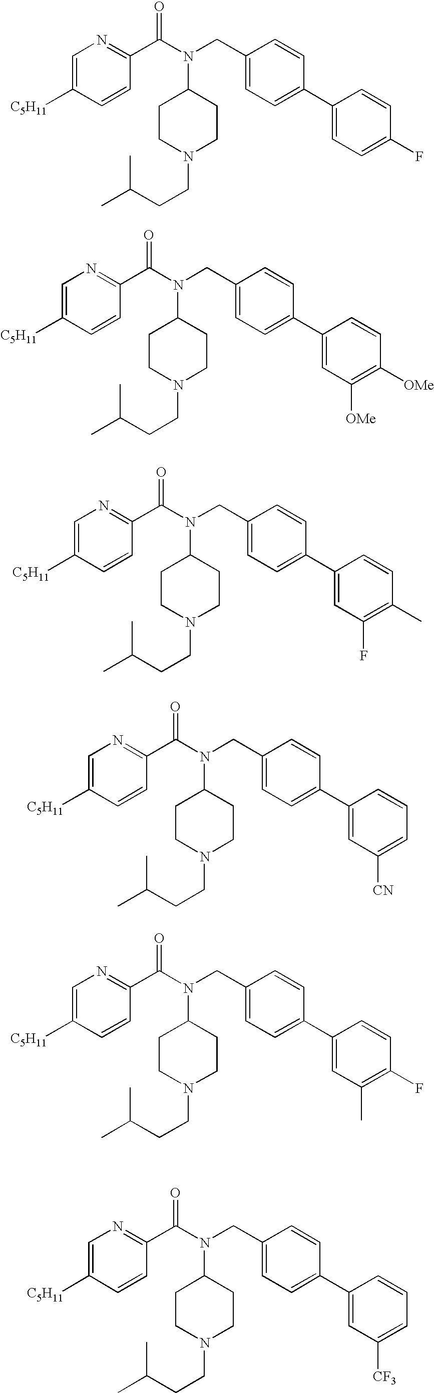

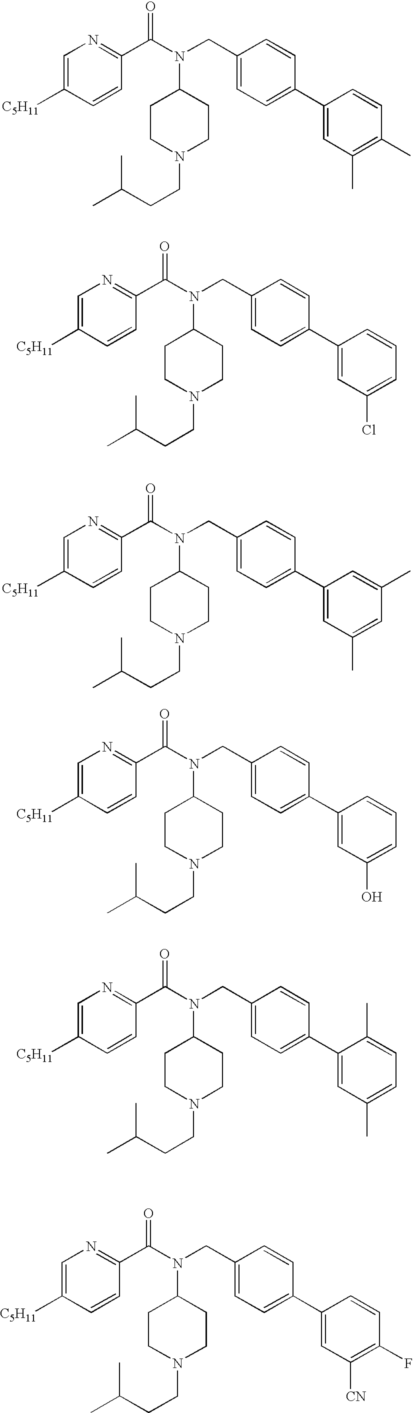

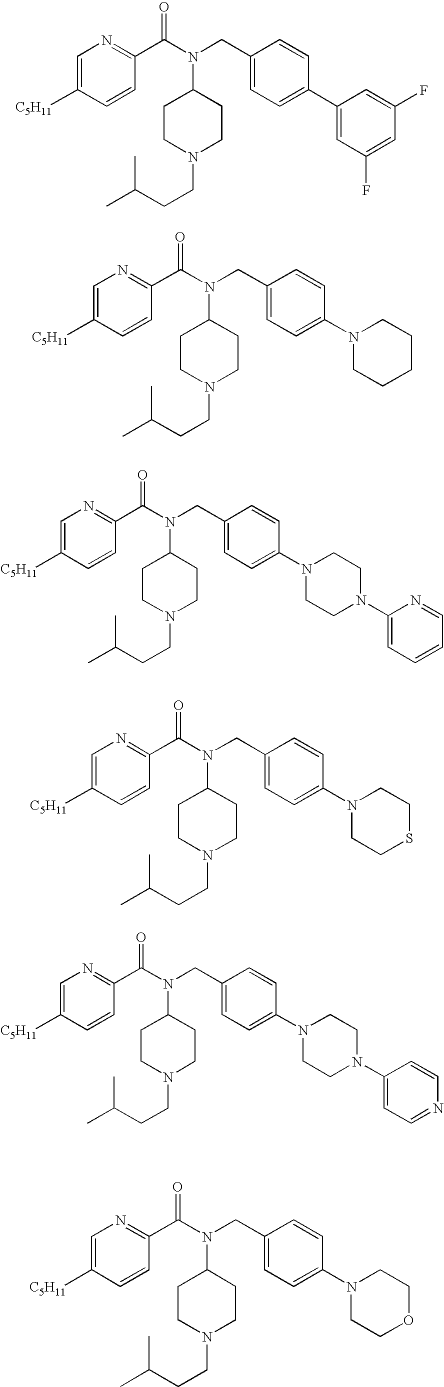

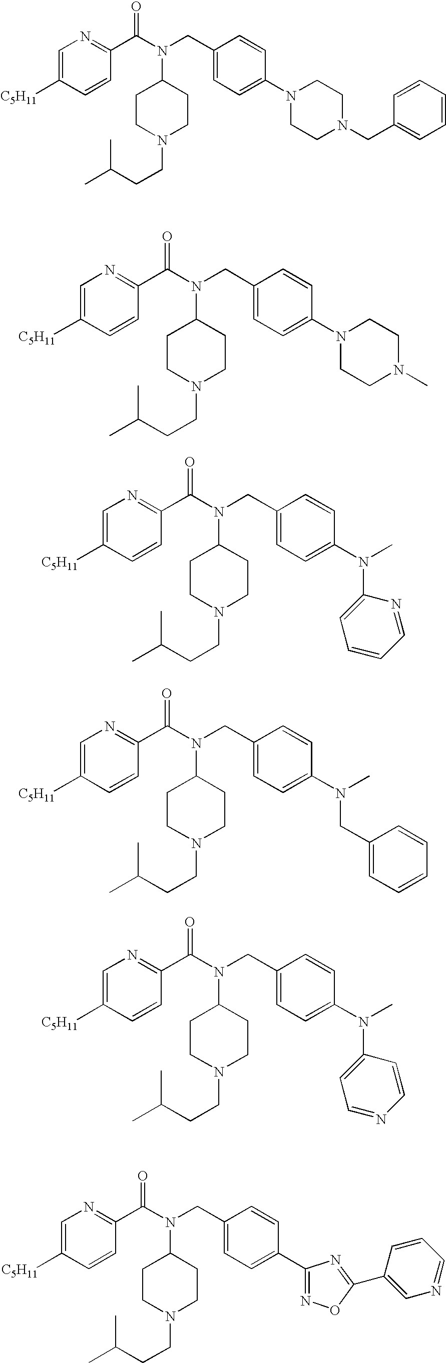

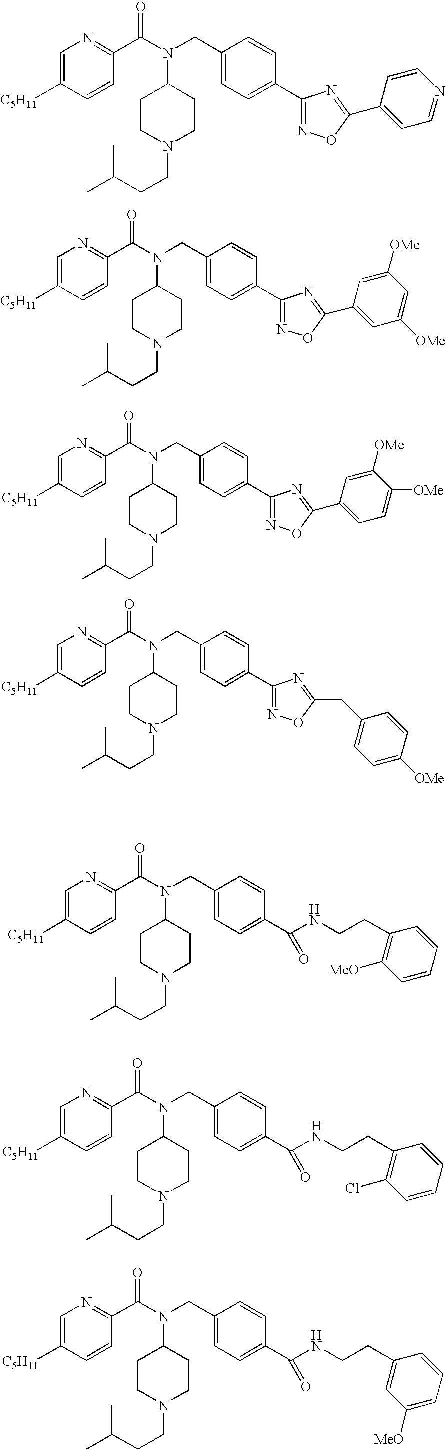

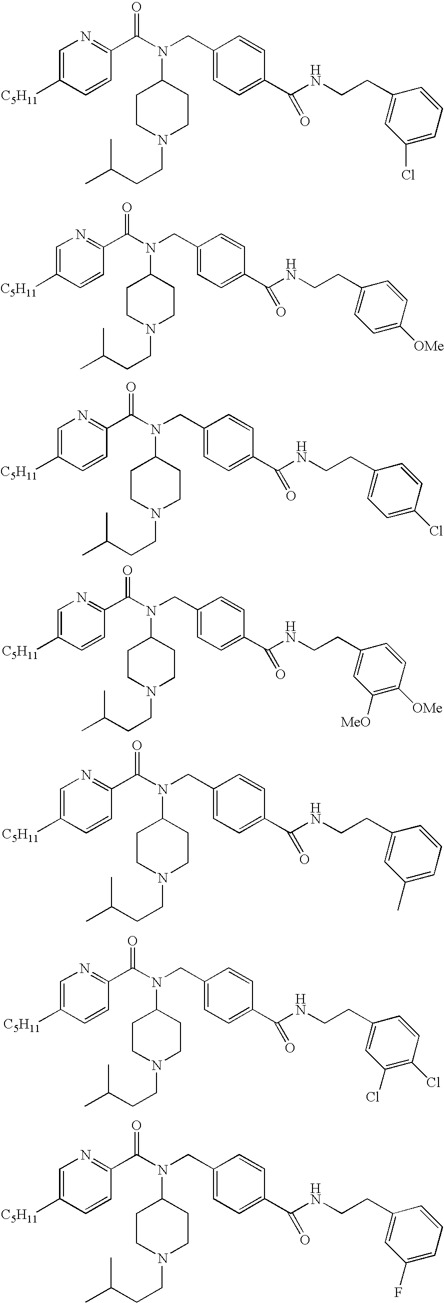

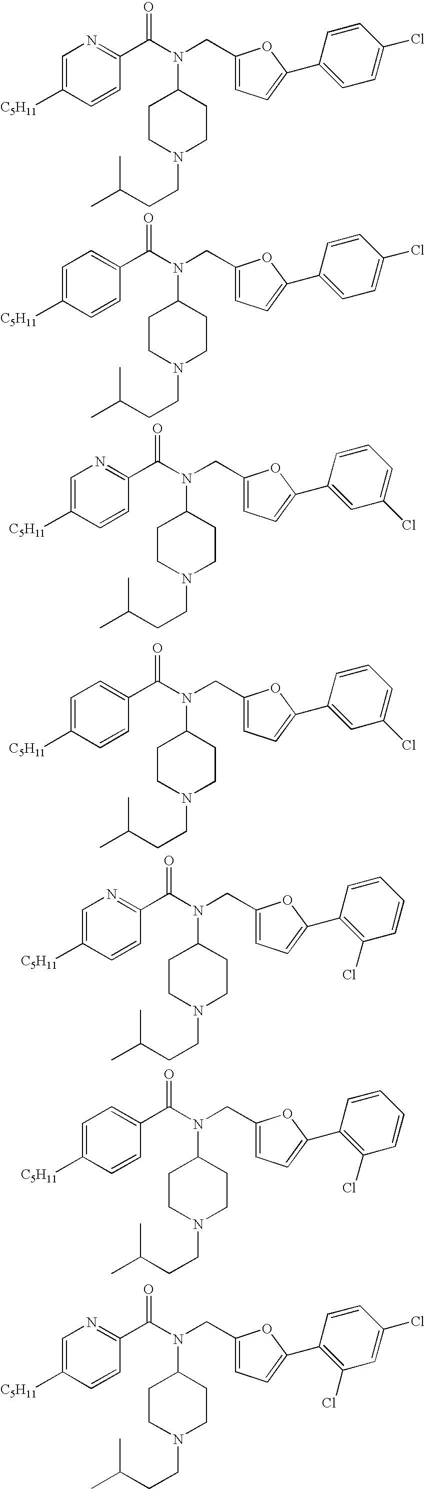

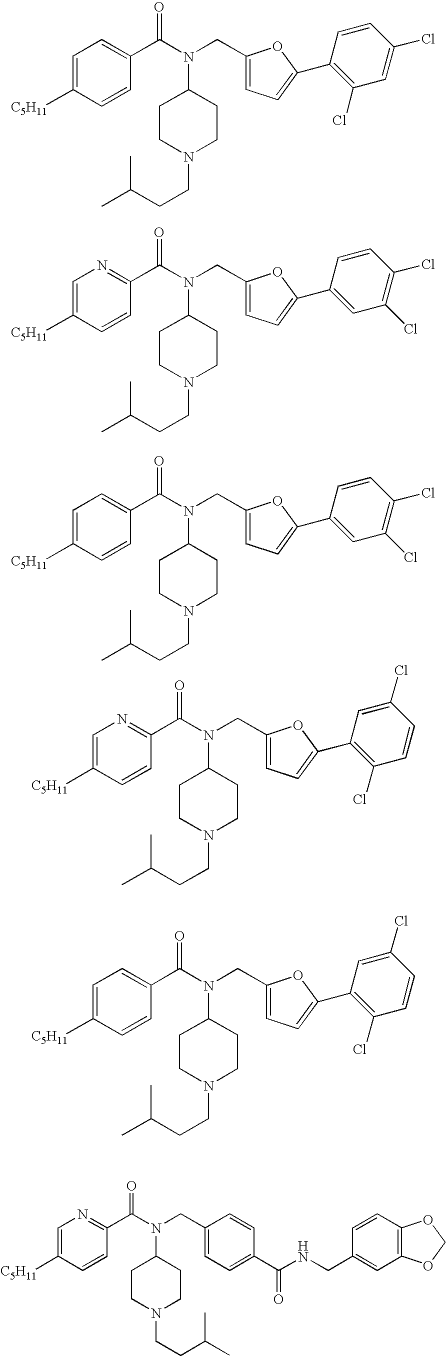

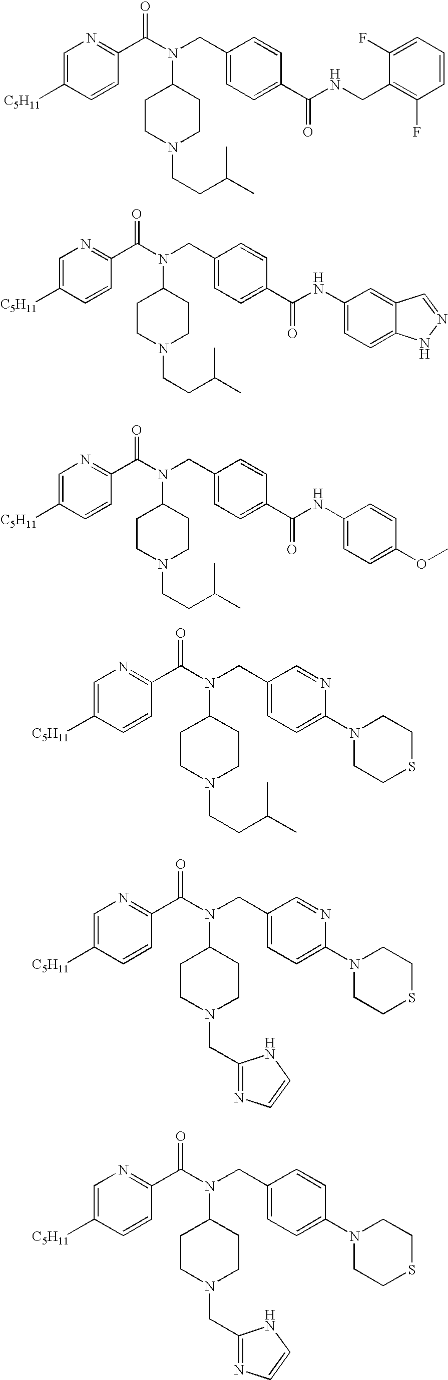

The compounds of the formula I are useful for the treatment and/or prevention of diseases demanding the inhibition of parasite aspartic proteases, such as especially plasmepsin II and/or plasmepsin IV. In particular, the compounds of the formula I are useful for the treatment and/or prevention of protozoal infections, especially in the treatment and/or prevention of malaria, in particular plasmodium falciparum malaria.

In one embodiment, the invention relates to a method for the treatment and/or prevention of the diseases mentioned herein, especially malaria, said method comprising administering to a subject a pharmaceutically active amount of a compound of formula I.

A further aspect of the present invention relates to pharmaceutical compositions comprising a compound of formula I and a pharmaceutically acceptable carrier material. These pharmaceutical compositions may be used for the treatment and/or prevention of the above-mentioned diseases. The pharmaceutical compositions can be used for enteral, parenteral, or topical administration. They can be administered, for example, perorally, e.g. in the form of tablets, coated tablets, dragées, hard and soft gelatine capsules, solutions, emulsions or suspensions, nasal, e.g. in the form of sprays, rectally, e.g. in the form of suppositories, parenterally, e.g. in the form of injection solutions or infusion solutions, or topically, e.g. in the form of ointments, creams or oils.

The invention also relates to the use of a compound of formula I for the preparation of pharmaceutical compositions for the treatment and/or prevention of the above-mentioned diseases.

The production of the pharmaceutical compositions can be effected in a manner which will be familiar to any person skilled in the art (see for example Mark Gibson, Editor, Pharmaceutical Preformulation and Formulation, IHS Health Group, Englewood, Colo., USA, 2001; Remington, The Science and Practice of Pharmacy, 20th Edition, Philadelphia College of Pharmacy and Science). In particular, the pharmaceutical compositions may contain the compounds of formula I or their pharmaceutically acceptable salts in combination with inorganic and/or organic excipients which are usual in the pharmaceutical industry like lactose, maize or derivatives thereof, talcum, stearinic acid or salts of these materials.

For gelatine capsules vegetable oils, waxes, fats, liquid or half-liquid polyols etc. may be used. For the preparation of solutions and syrups e.g. water, polyols saccharose, glucose etc. are used. Injectables are prepared by using e.g. water, polyols, alcohols, glycerin, vegetable oils, lecithin, liposomes etc. Suppositories are prepared by using natural or hydrogenated oils, waxes, fatty acids (fats), liquid or half-liquid polyols etc.

The compositions may contain in addition preservatives, stability improving substances, viscosity improving or regulating substances, solubility improving substances, sweeteners, dyes, taste improving compounds, salts to change the osmotic pressure, buffer, anti-oxidants etc.

The compounds of formula I or the above-mentioned pharmaceutical compositions may further also be used in combination with one or more other therapeutically useful substances e.g. with other antimalarials like quinine, chloroquine, amodiaquine, mefloquine, primaquine, tafenoquine, artemisinin and artemisinine-derivatives like artemether, arteether or artesunat, pyrimethamine-sulfadoxine (Fansidar), mepacrine, halofantrine, proguanil, chloroproguanil, lumefantrine, pyronaridine, atovaquone and the like and/or antibiotics like rifampicine, doxycycline, clindamycine or azithromycine and the like.

The dosage of a compound of formula I may vary within wide limits but should be adapted to the specific situation. In general the dosage given in oral form should daily be between about 3 mg and about 3 g, preferably between about 10 mg and about 1 g, especially preferred between 5 mg and 300 mg, per adult with a body weight of about 70 kg. The dosage should be administered preferably in 1 to 3 doses per day which are of equal weight. As usual, children should receive lower doses which are adapted to body weight and age.

The present invention also relates to pro-drugs of a compound of formula I that convert in vivo to the compound of formula I as such. Any reference to a compound of formula I is therefore to be understood as referring also to the corresponding pro-drugs of the compound of formula I, as appropriate and expedient.

The compounds of the formula I of the present invention can be prepared according to the sequences of reactions outlined below in Schemes 1 to 10. (for simplicity and clarity reasons, only parts of the synthetic possibilities which lead to compounds of formulae I to VII are described). The Schemes are structured according to the different structural classes of the compounds of formula I. All chemical transformations can be performed according to well-known standard methodologies as described in the literature or as described in the preparation of certain specific examples.

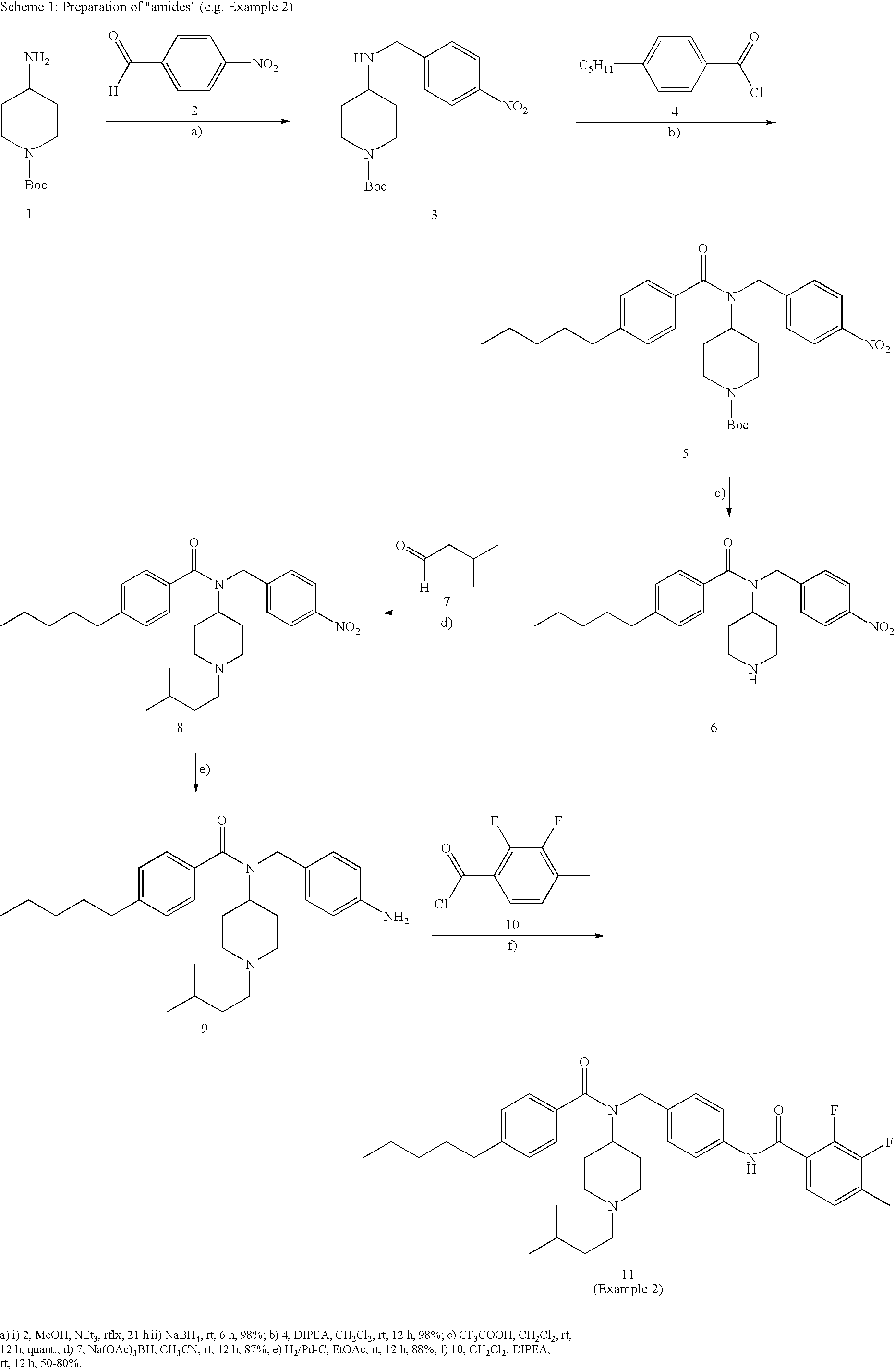

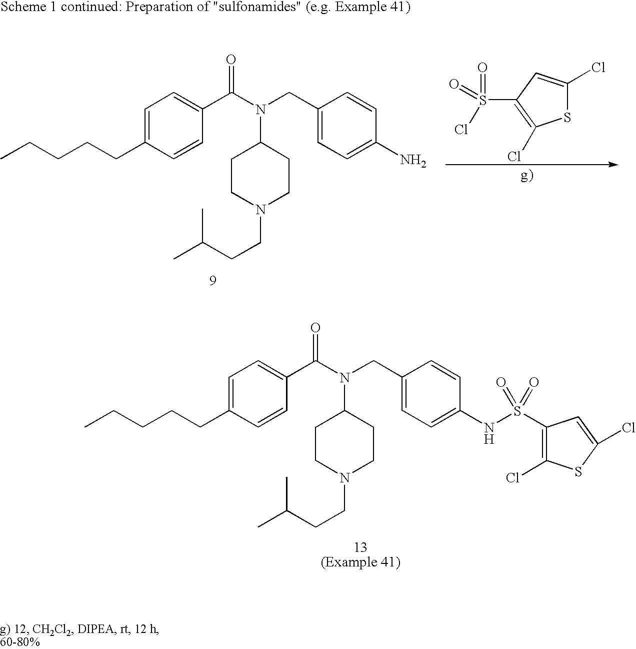

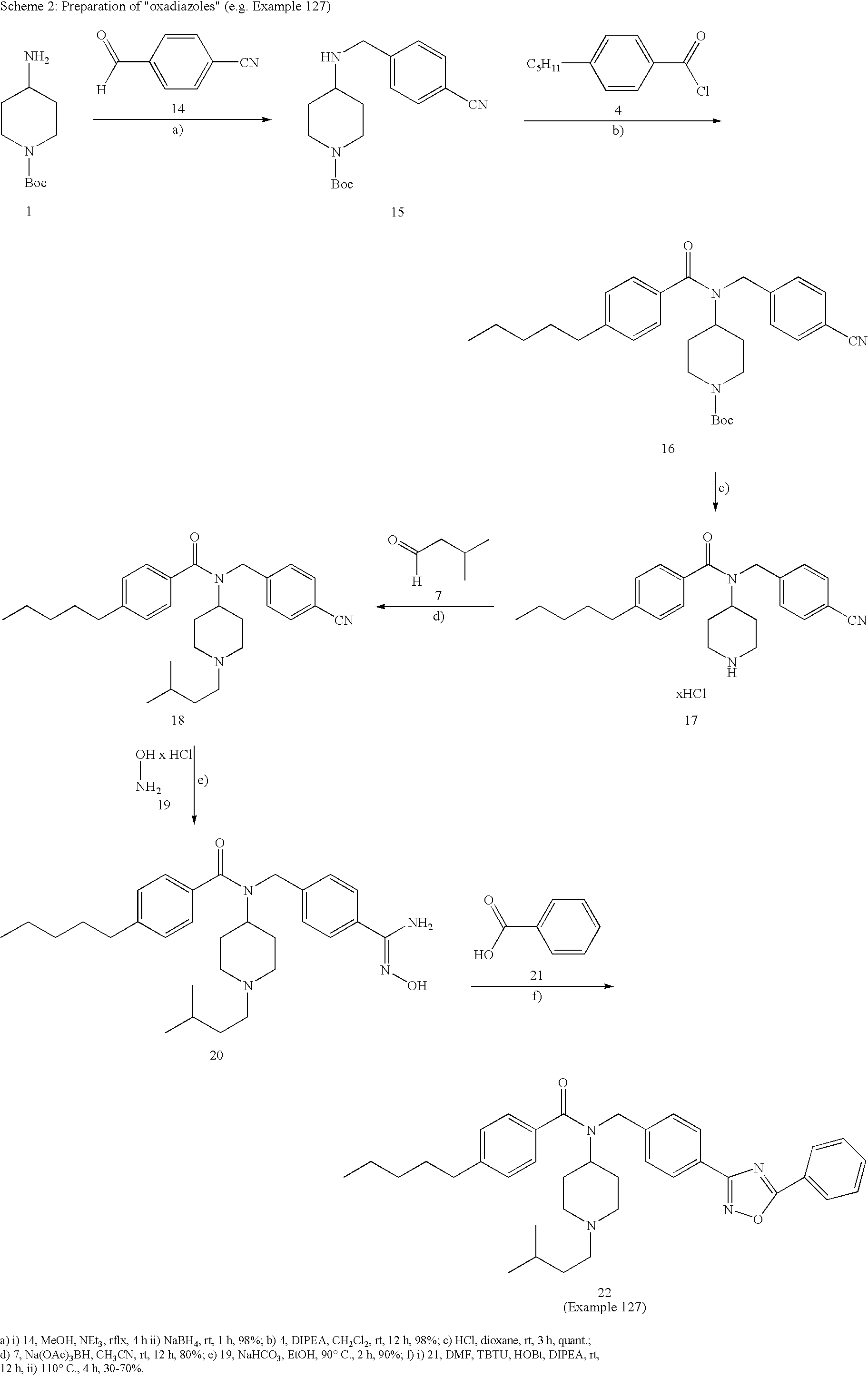

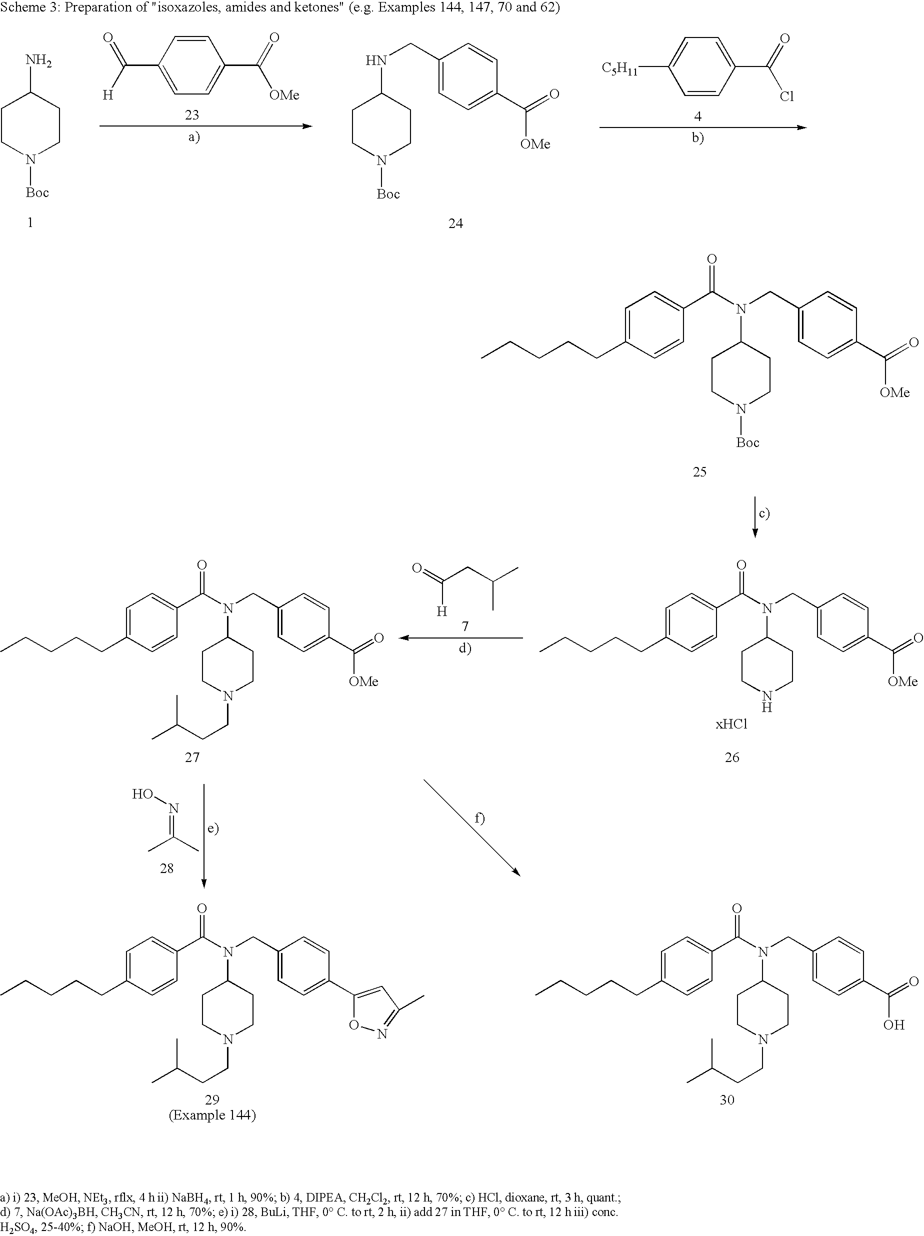

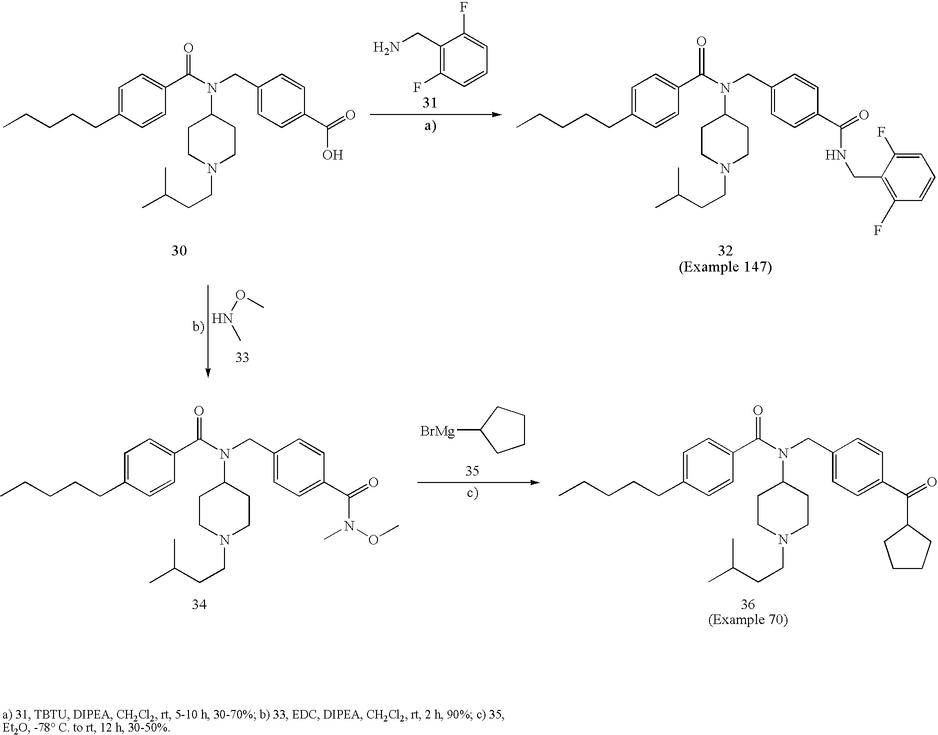

According to the sequence given above, derivatives 37 and 38 (meta substitution) are as well accessible: 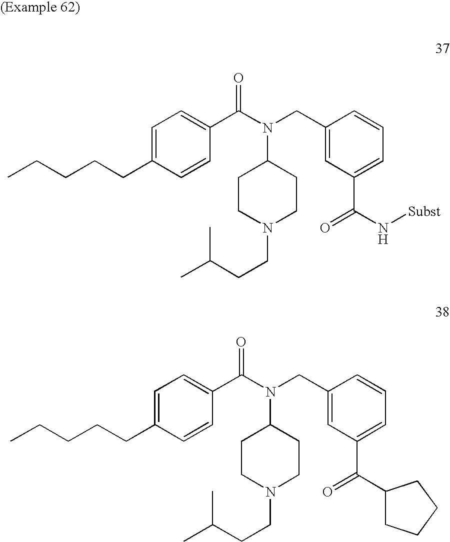 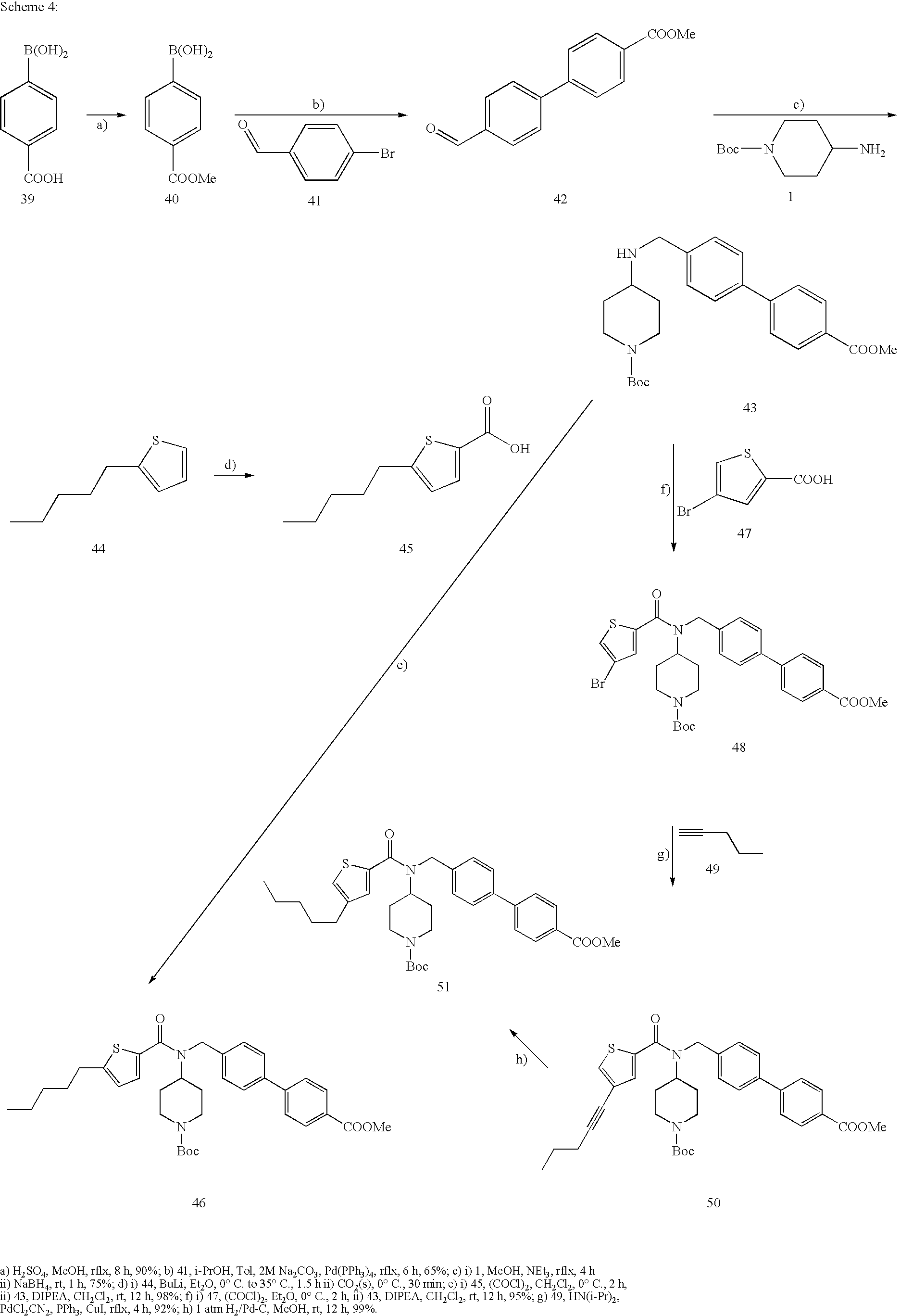 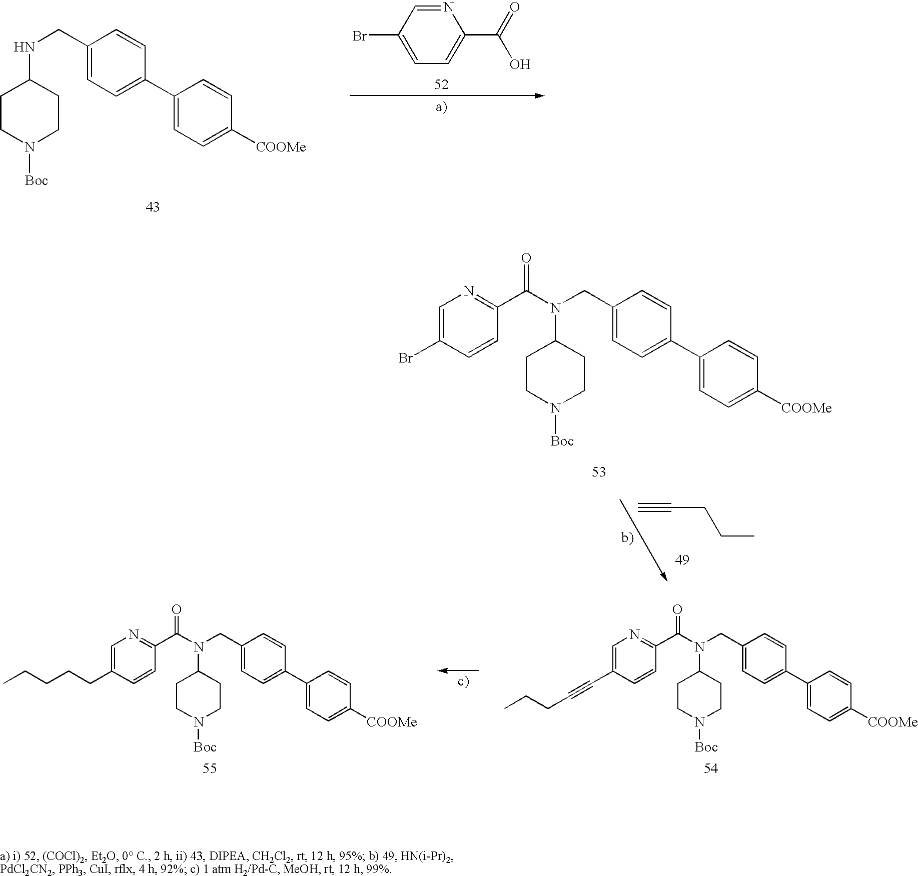 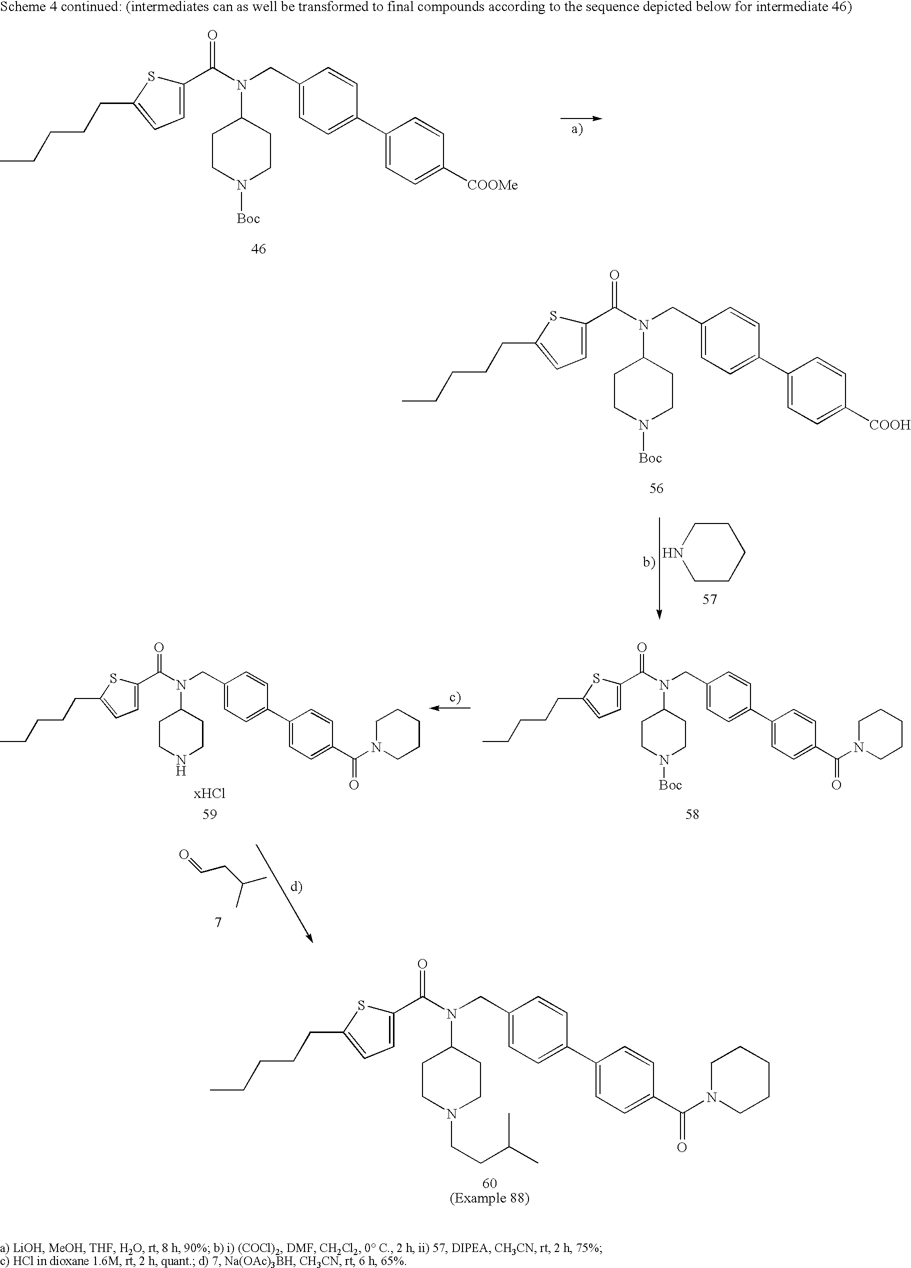 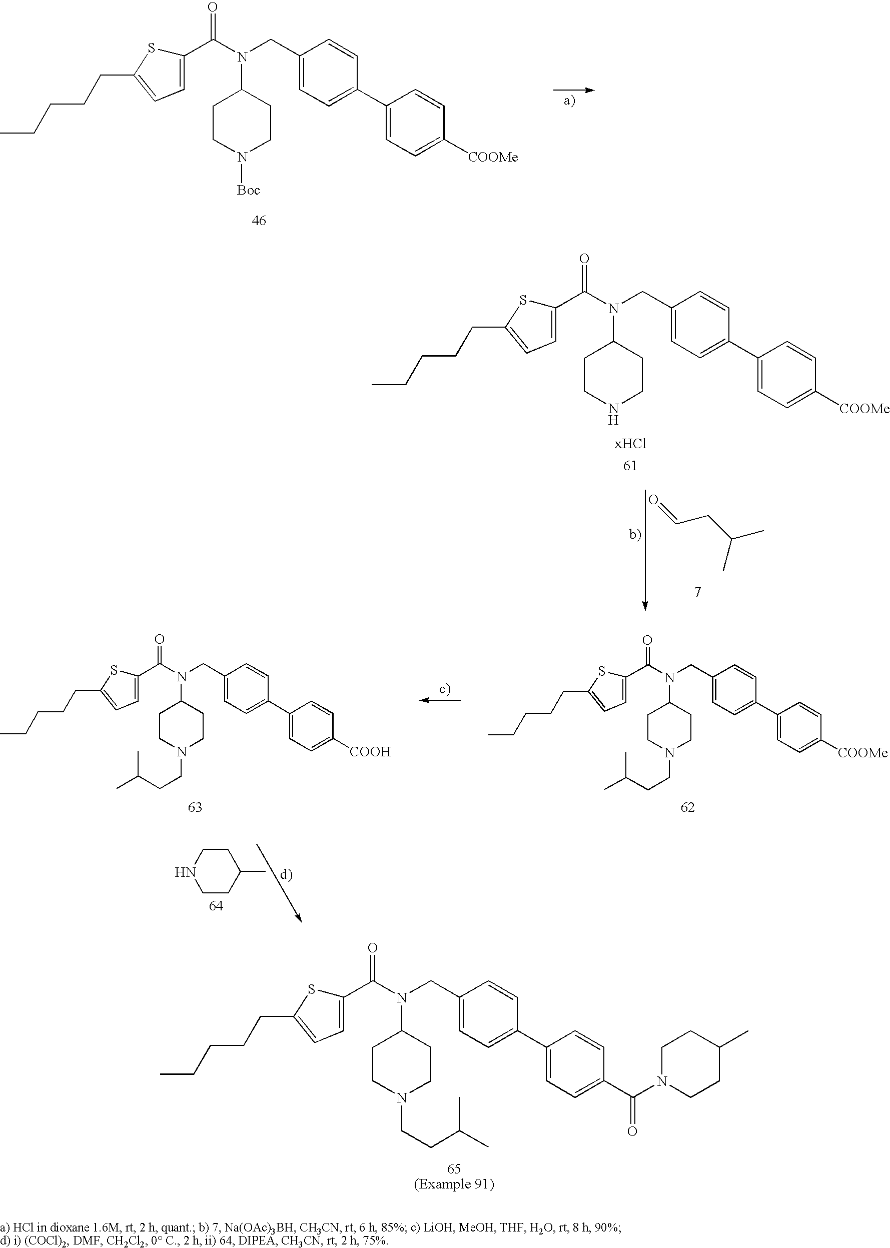 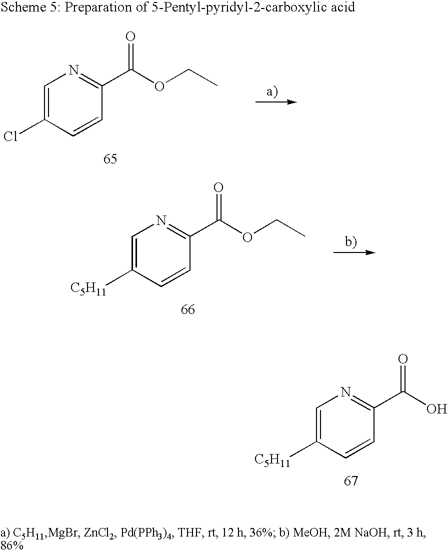 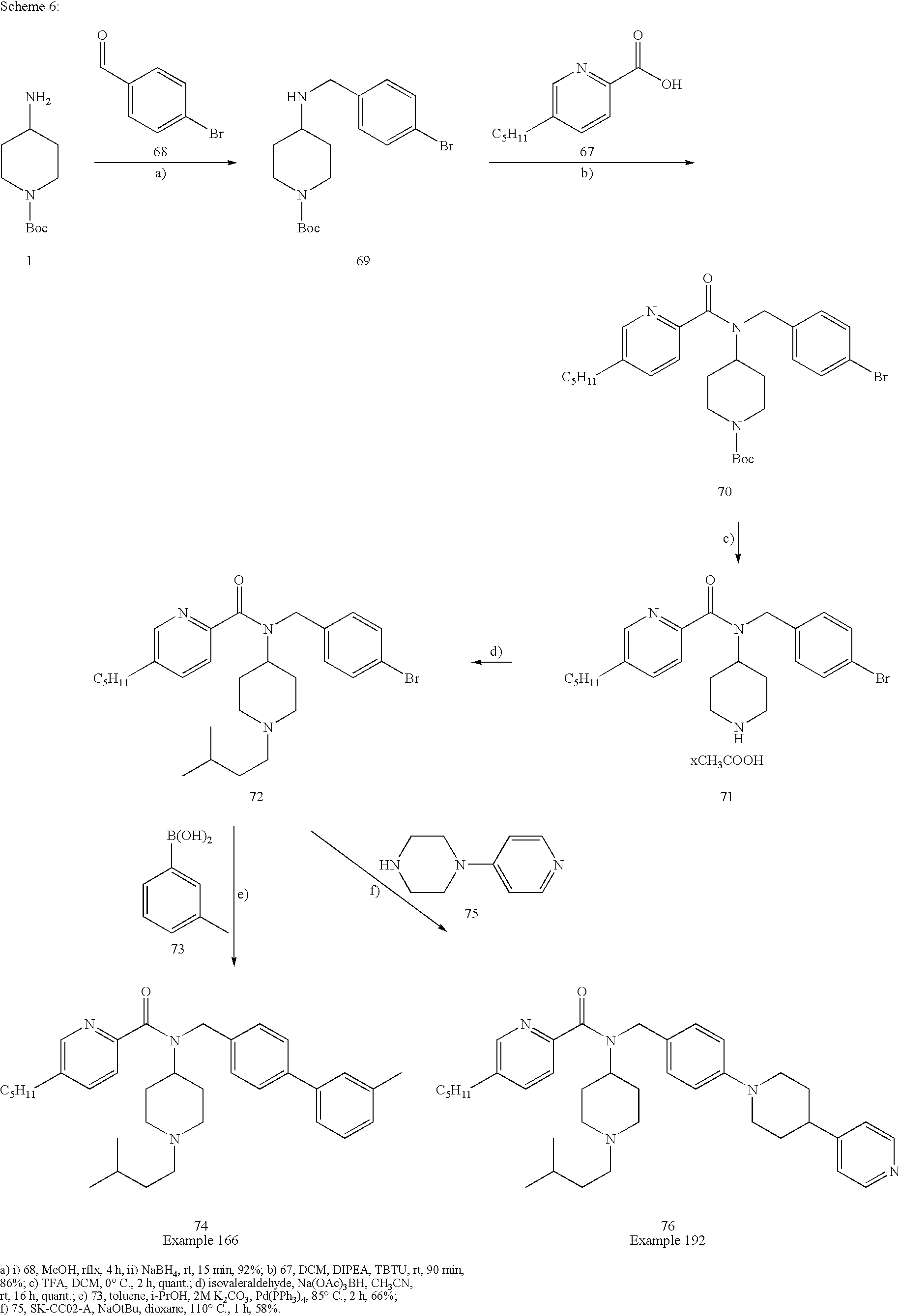 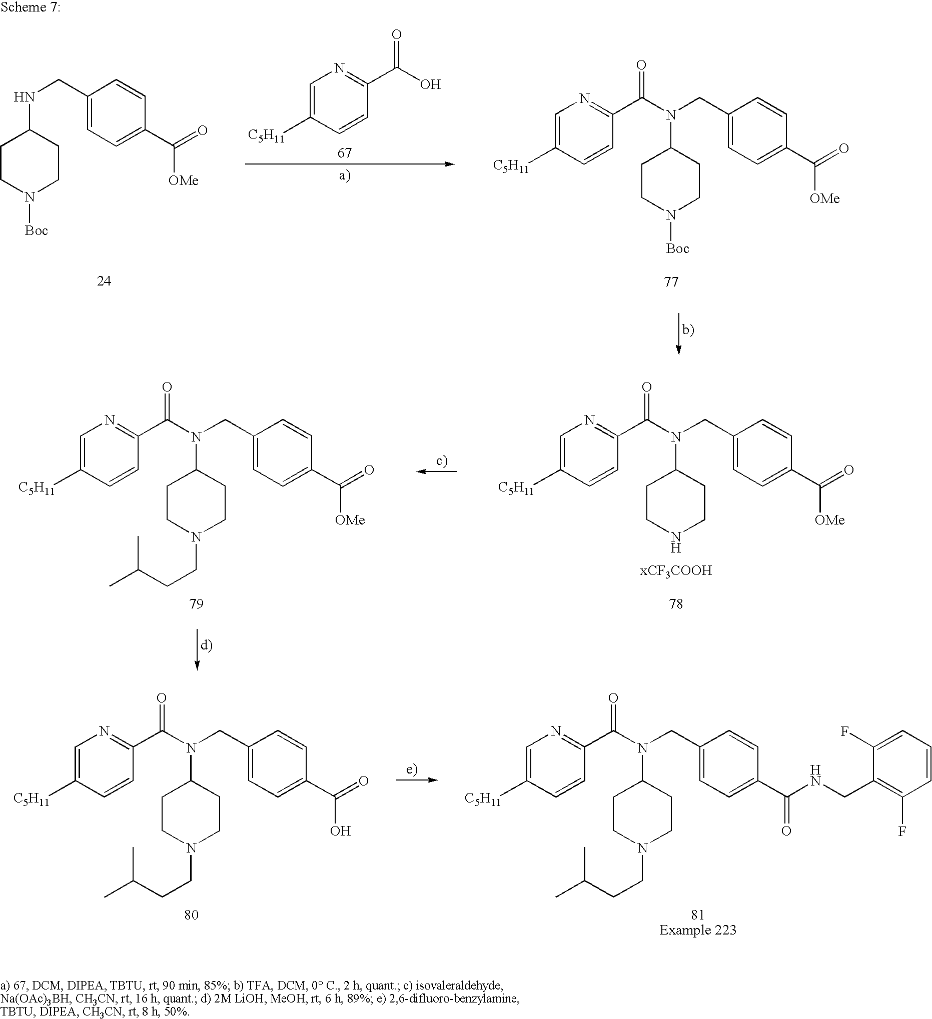 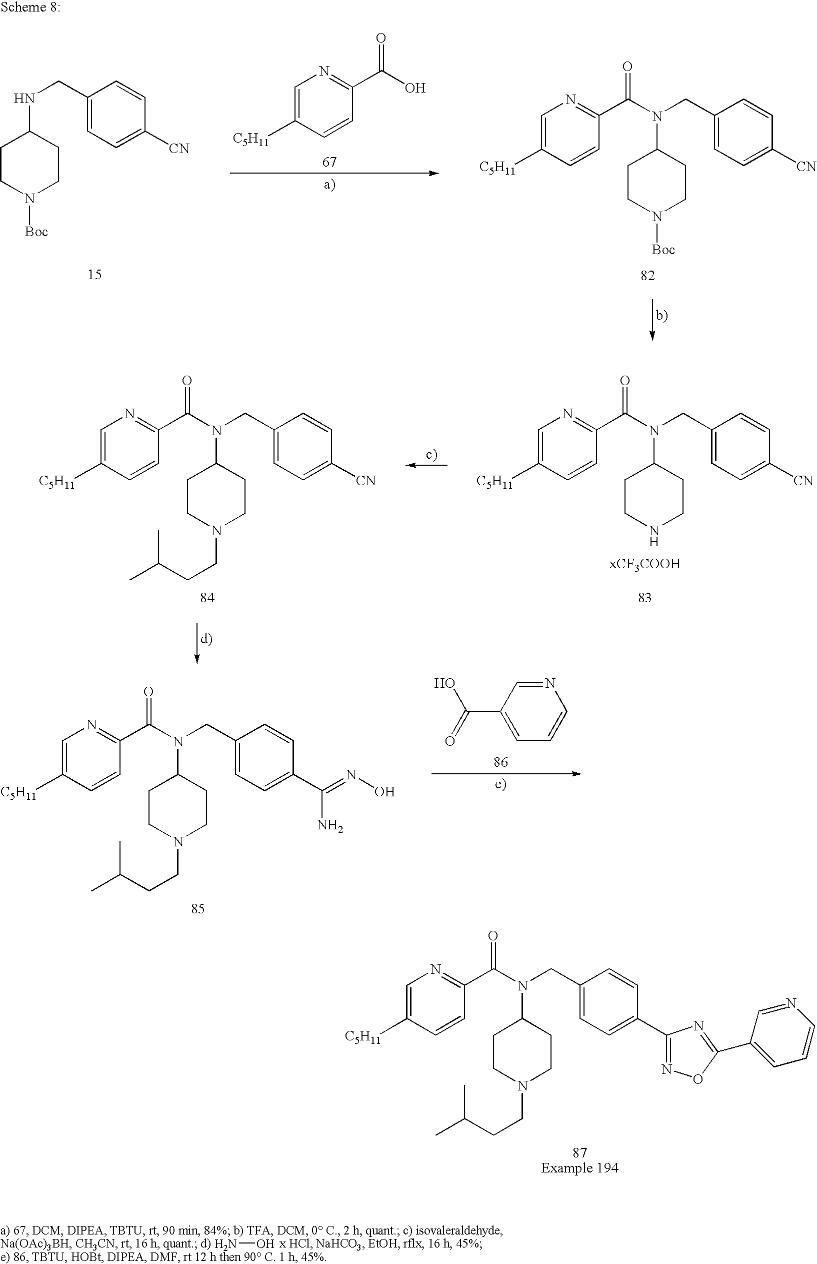 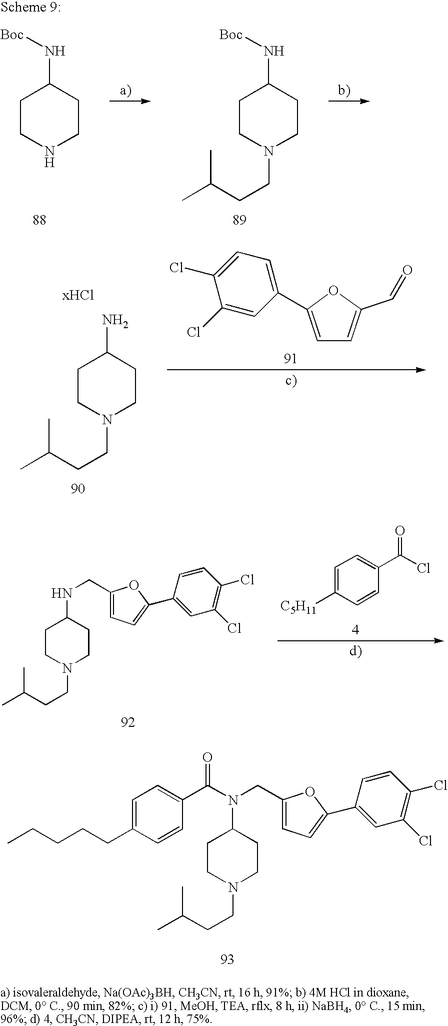 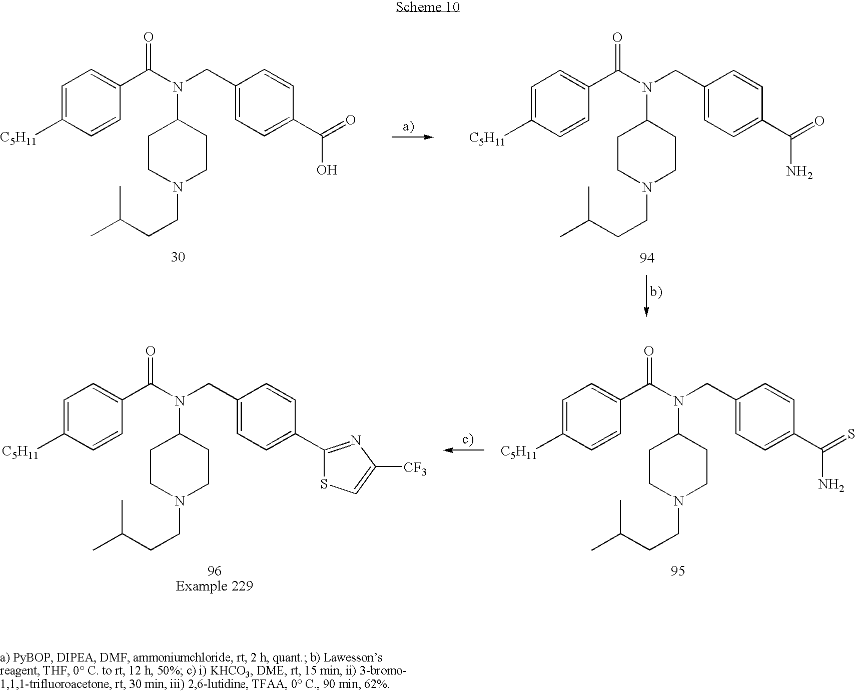
Notes to Schemes 1 to 10:

Boc-4-aminopiperidine (1) is commercially available from Neosystems.

The reductive aminations with sodium borohydride as the reducing agent as well as the acylations with acid chlorides were performed as described in Mueller, R. et al., Molecules, 2003, 8, 556-564.

Boc-deprotection generally was achieved by stirring compounds in 4 M HCl in dioxane for 1 h at room temperature followed by evaporation to dryness [T. W. Greene, P. G. M. Wuts, Protective groups in organic synthesis, Wiley-Interscience, 1991; P. J. Kocienski, Protecting Groups, Thieme, 1994; Mueller, R. et al., Molecules, 2003, 8, 556-564].

Reductive amination with sodium triacetoxyborohydride was performed as described in Mueller, R. et al., Molecules, 2003, 8, 556-564; Abdel-Magid, A. F. et al., J. Org. Chem., 1996, 61, 3849-3862.

Acylations with sulfonylchlorides were performed in analogy to the acylations with acid chlorides.

Condensations of carboxylic acids with amines were performed with the help of a condensation reagent (examples of such reagents given in Novabiochem 2004/05 Catalog, p. 353-373, procedures according to the references cited there with the respective reagent).

Aromatic nitro-group reduction to the aniline functionality was performed as described in the detailed synthetic sequence of Example 2 in Scheme 1.

Weinreb amide chemistry for the synthesis of ketones was performed according to procedures described in B. Chen et al, J. Org. Chem., 2003, 68, 4195-4205; J. H. Chan et al, J. Med. Chem., 2004, 47, 1175-1182; F. A. David et al, Org. Lett., 2003, 5, 3856-3857.

Isoxazole synthesis from carboxylic esters was performed according to procedures described in F. I. Carroll et al, J. Med. Chem., 2004, 47, 296-302; J. R. Malpass et al, J. Org. Chem., 2004, 69, 5328-5334; J. M. Malpass et al, J. Org. Chem., 2003, 68, 9348-9355.

Oxadiazole synthesis from nitriles was performed according or in analogy to procedures given in the following papers: A. Hamze et al, J. Org. Chem., 2003, 68, 7316-7321; E. Meyer et al, Synthesis, 2003, 899-905; Y. Huang et al, Bioorg. Med. Chem., 2001, 9, 3113-3122; G.-D. Zhu et al, J. Med. Chem., 2001, 44, 3469-3487.

Esterhydrolysis was performed according or in analogy to procedures described in B. Jaun et al, Liebigs Ann./Recueil, 1997, 1697-1710.

Sonogashira couplings were performed according to S. Thorand et al, J. Org. Chem., 1998, 63, 8551-8553; D. Trachsel, Helv. Chim. Acta, 2003, 86, 2754-2759; G. Reginato et al, J. Org. Chem., 1997, 62, 6187-6192; J. Dogan et al, Heterocycles, 1995, 41, 1659-1666; C. Dhih et al, J. Med. Chem., 1992, 35, 1109-1116; U. Dahlmann et al, Helv. Chim. Acta, 1996, 79, 755-766; J. J. Song et al, J. Org. Chem., 2001, 66, 605-608.

Reductions of triple bonds to single bonds were performed in ethanol with 10% Pd—C as the catalyst and at 2 to 5 bar hydrogen pressure for 2 to 6 h.

Suzuki couplings to biaryl-systems were performed according to the procedure described in C. Boss et al., Curr. Med. Chem., 2003, 10, 886-907 and reference [57] cited there.

The aryl-amination reactions were usually performed in an inert atmosphere (Argon or N₂-gas) with a suitable catalyst like SK-CC01-A or SK-CC02-A [commercially available from Solvias AG or eventually Fluka and especially designed for aryl-aminations, see Anita Schnyder et al., Angew. Chem. Int. Ed., 2003, 41, 3668-3671; Ricci, A. (Editor); Modern Amination Methods; Wiley-VCH, Germany, 2000, especially Chapter 7, pp 195-262 and references cited there].

5-Chloro-2-ethoxycarbonyl-pyridin (65) was prepared from 2,5-dichloropyridin by Solvias AG, Basel via a procedure described in Heterocycles, 1999, 51, 11, p 2589. Negishi reaction for the introduction of the C₅-chain was performed according to procedures described in e.g. WO 03/093267.

Esterhydrolyses were performed according to a procedure described in Liebigs Ann./Recueil, 1997, 1697-1710.

Thiazole synthesis (see scheme 10) was performed according to procedures described in Tetrahedron, 1997, 53, 8149-8154; Tetrahedron Lett., 1995, 36, 5057-5060, Angew. Chem. Int. Ed. Engl., 1996, 35, 1503-1506, and Synth. Commun., 1990, 20, 2235-2249.

The following examples illustrate the invention but do not limit the scope thereof. All temperatures are given in ° C.

Abbreviations (as used herein):
aq. aqueous
Boc or boc tert.-butyloxycarbonyl
BSA Bovine Serum Albumin
BuLi n-Butyllithium
conc. concentrated
DCM dichloromethane
DIPEA di-isopropyl-ethyl-amine (Hünigs base)
DME 1,2-dimethoxyethane
DMF dimethylformamide
DMSO dimethylsulfoxide
EDC N-(3-dimethylaminopropyl)-N′-ethylcarbodiimide
ELSD evaporative light-scattering detection
eq. equivalent(s)
Et ethyl
EtOAc ethyl acetate
Ex. example
h hour(s)
HOBt N-hydroxybenzotriazole mono-hydrate
HPLC High Performance Liquid Chromatography
HV High Vacuum
i-Pr isopropyl
i-PrOH isopropanol
LC-MS Liquid Chromatography-Mass Spectroscopy
MeOH methanol
min minute(s)
N₂ nitrogen as protective gas/inert atmosphere
NEt₃ triethylamine
NMM N-methyl-morpholine
PBS phosphate buffered saline
Ph phenyl
PM plasmepsin
PyBOP benzotriazole-1-yl-oxy-tris-pyrrolidino-phosphonium hexafluorophosphate
quant. quantitative
rflx reflux
rt room temperature
(s) solid
sat. saturated
SK-CC02-A Chloro(di-2-norbornyl-phosphino) (2-dimethyl-amino-methylferrocen-1-yl) palladium (III)
Subst substituent
TBTU 2-(1H-benzotriazole-1-yl)-1,1,3,3-tetramethyluronium tetrafluoroborate
t-Bu tert.-butyl
TEA triethylamine
TFA trifluoroacetic acid
TFAA trifluoroacetic anhydride
THF tetrahydrofuran
TLC thin layer chromatography
Tol toluene
t_{R} retention time (in minutes)
UV ultra violet
wt % weight percentage

### GENERAL PROCEDURES AND EXAMPLES

All solvents were stored over molecular sieves. All reagents were used without further purification as received from commercial sources.

All compounds were characterized by ¹H-NMR (300 MHz) and occasionally by ¹³C-NMR (75 MHz) (Varian Oxford, 300 MHz), by LC-MS (Finnigan AQA/HP 1100; Column: Develosil C30 Aqua, 50×4.6 mm, 5 μm; Gradient: 5-95% acetonitrile in water, 1 min, with 0.03% TFA, flow: 4.5 ml/min), by TLC (TLC-plates from Merck, Silica gel 60 F₂₅₄).

Preparative HPLC-System: Column: Zorbax SB-AQ 5 mM, 21.2×50 mm; flow: 40 ml/min; Gradient: 10-95% acetonitirle in water, 3.5 min, with 0.5% formic acid; detection by UV/ELSD.

a) Typical Procedures:

Typical procedure A) for the Reductive Amination:

The amine and the aldehyde (0.97 eq.) (which are used as starting materials, are known compounds), are mixed in anhydrous MeOH and stirred under reflux for 4 h. The reaction mixture is cooled to rt followed by the addition of sodium borohydride (1.5 eq.). Stirring is continued for 15 min. Small amounts of water are carefully added and the methanol is removed under reduced pressure. Water is added to the residue which is then extracted 3× with EtOAc. The combined organic layers are washed with brine, dried over sodium sulfate, filtered and the solvent is evaporated. The secondary amine is usually obtained in high purity and can be used in subsequent transformations without further purification. In case purification seems to be necessary, either flash chromatography over silica gel with solvent mixtures like DCM/MeOH=9/1 or HPLC-purifications were performed.

Typical Procedure B) for the Acylation:

To a solution of the amine in anhydrous EtOAc (or acetonitirle or DCM) is added a base like NEt₃ (or DIPEA, or NMM) followed by the addition of the carboxylic acid chloride (1.2 eq.). The reaction mixture is stirred for 2 to 14 h at rt, followed by standard aqueous work-up and purification, either by flash chromatography over silica gel with an appropriate solvent mixture (usually EtOAc/hexane) or by HPLC, to give the amide intermediate.

The carboxylic acid chlorides {R₁—(CO)—Cl} may be obtained in situ from the corresponding carboxylic acid as described in the literature (i.e.: Devos, A., Rémion, J., Frisque-Hesbain, A.-M., Colens, A., Ghosez, L., J. Chem. Soc., Chem. Commun. 1979, 1180).

Typical Procedure C) for the Boc-Deprotection:

The Boc-protected intermediate is dissolved in dioxane followed by the addition of 4M HCl in dioxane (commercially available from Aldrich) at rt. Stirring is continued for 1 to 2 h. The reaction mixture is evaporated to dryness. In case of very sensitive intermediates, the Boc-protected compound is dissolved in DCM followed by the addition of TFA at rt. Stirring is usually continued for 3 to 4 h followed by evaporation to dryness [Kocienski, P. J., Protecting Groups, Thieme Verlag Stuttgart, 1994; Greene, T. W., Wuts, P. G. M., Protective Groups in Organic Synthesis, Wiley-Interscience, 2^{nd} Edition, 1991].

Typical Procedure D) for the Second Reductive Amination:

The amine and the aldehyde (1.5 eq.) are mixed in anhydrous dichloromethane (or THF, or acetonitrile) and sodium triacetoxyborohydride (1.3 eq.) is added. After stirring the solution for 48 h, methanol is added and the reaction mixture is treated in the same manner as described in procedure A).

Typical Procedure E) for the Aryl/Heteroaryl-Amination Reaction:

[see also: Ricci, A. (Editor), Modern Amination Methods, Wiley-VCH, Germany, 2000, especially Chapter 7, pp 195-262 and references cited there]

In a dry reaction flask, toluene is degassed for 30 min with N₂. The aryl-halogenide or the heteroaryl-halogenide, the amine and sodium tert.-butoxide are added. The mixture is heated to 100° C. for 30 min followed by the addition of the appropriate palladium-catalyst (e.g. SK-CC01A or SK-CC02-A from Solvias AG; M. Thommen et al., sp2, September 2003, p 32-35, and references cited therein) suspended in toluene. Stirring at 10° C. was continued for 2 to 8 h followed by standard aqueous work up and purification of the compounds by preparative TLC or by HPLC.

All chemical transformations can be performed according to well known standard methodologies as described in the literature or as described in the typical procedures above or according to further procedures given below in the detailed description for the preparation of Example 2, Example 70, Example 127, Example 144, Example 166, Example 192, Example 194, Example 220, Example 223, Example 231 and some precursors. All compounds described as examples can be prepared by the appropriate combination of the described procedures, the literature procedures and the choice of the appropriate starting materials by the person skilled in the art of organic synthesis.

### Synthesis of N-[1-(3-Methyl-butyl)-piperidin-4-yl]-4-pentyl-N-(4-(2,3-difluoro-4-methyl-benzoyl)-amino-benzyl)-benzamide

### EXAMPLE 2

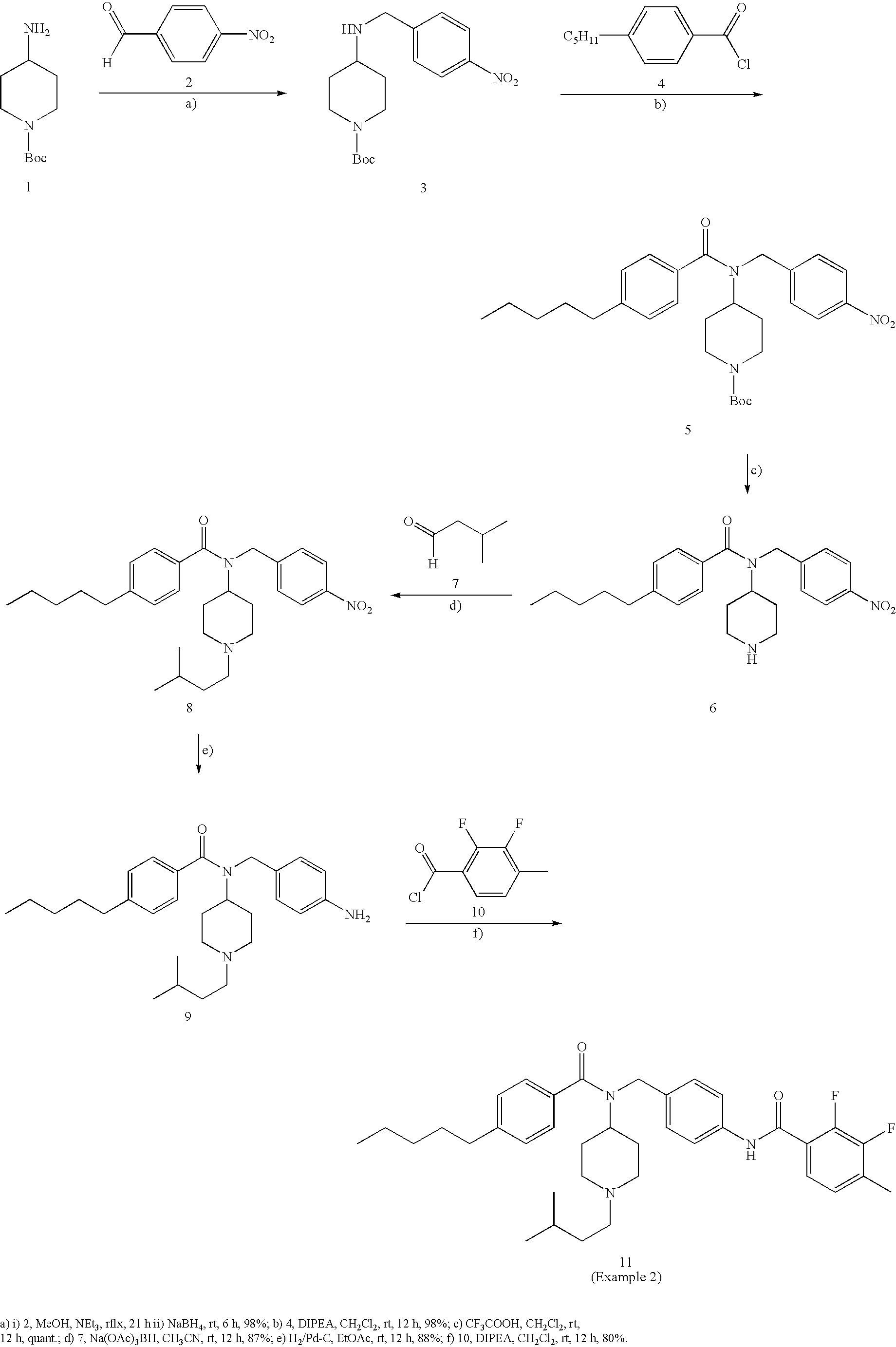

4-(4-Nitro-benzylamino)-piperidine-1-carboxylic acid tert-butyl ester (3): A solution of 4-amino-N-Boc-piperidine hydrochloride (1) (5.0 g, 21.12 mmol), 4-nitrobenzaldehyde (2) (3.19 g, 21.12 mmol) and triethylamine (2.9 ml, 21.12 mmol) was refluxed in methanol (100 ml) for 21 h followed by the addition of sodium borohydride (1.28 g, 33.79 mmol) at rt. Stirring was continued for 6 h. Saturated sodium bicarbonate solution was added to the reaction mixture and the product was extracted with ethyl acetate (3×100 ml). The combined organic layers were washed with brine, dried over magnesium sulfate, filtered and concentrated under reduced pressure to give 7.2 g (98%) of 3 as an orange oil.

4-[(4-Nitro-benzyl)-(4-pentyl-benzoyl)-amino]-piperidine-1-carboxylic acid tert-butyl ester (5): Compound 3 (7.65 g, 22.81 mmol) was dissolved in dichloromethane (530 ml) followed by the addition of Hünigs base (DIPEA, 8.84 g, 68.43 mmol) and the slow addition of 4-pentylbenzoyl chloride (4) (4.81 g; 22.81 mmol). The reaction mixture was stirred at rt for 12 h, poured onto saturated sodium bicarbonate solution and the organic phase was separated, washed with brine, dried over magnesium sulfate, filtered and concentrated under reduced pressure to give 12 g (98%) of 5 as an orange glassy solid.

N-(4-Nitro-benzyl)-4-pentyl-N-piperidin-4-yl-benzamide (6): Compound 5 (12.09 g, 23.72 mmol) was dissolved in dichloromethane (270 ml) and trifluoroacetic acid (27 g, 237.2 mmol) was added. Stirring was continued for 12 h. The reaction mixture was concentrated in vacuo and the product was purified by flash chromatography (silica gel, dichloromethane/methanol=7/1) to give 10 g (quant.) of 6 as yellow foam.

N-[1-(3-Methyl-butyl)-piperidin-4-yl]-N-(4-nitro-benzyl)-4-pentyl-benzamide (8): Compound 6 (10 g, 24.8 mmol) was dissolved in acetonitrile (120 ml), followed by the addition of isovaleraldehyde (7) (2.57 g, 29.83 mmol) and sodium triacetoxyborohydride (8.43 g, 39.78 mmol). The reaction mixture was stirred at rt for 12 h. Water 200 ml was added and the product was extracted with dichloromethane (3×100 ml). The combined organic layers were washed with brine, dried over magnesium sulfate, filtered and concentrated under reduced pressure. The product was purified by flash chromatography (silica gel, dichloromethane/methanol=95/5) to give 10.34 g (87%) of 8 as an orange powder.

N-(4-Amino-benzyl)-N-[1-(3-methyl-butyl)-piperidin-4-yl]-4-pentyl-benzamide (9): Compound 8 (5 g, 10.42 mmol) was dissolved in ethyl acetate (170 ml) and Pd/C (1.87 g, 10% containing 50 wt % of water) was added. The reaction mixture was put under an atmosphere of hydrogen and vigorously stirred for 12 h at rt, subsequently filtered over celite and concentrated under reduced pressure. The product was purified by flash chromatography (silica gel, dichloromethane/methanol=9.25/0.75) to give 4.1 g (88%) of 9 as a slightly brown solid.

N-[1-(3-Methyl-butyl)-piperidin-4-yl]-4-pentyl-N-(4-(2,3-difluoro-4-methyl-benzoyl)amino-benzyl)-benzamide (11): The following reaction was performed in a parallel chemistry setting: Compound 9 (50 mg, 0.111 mmol) was dissolved in dichloromethane (2 ml) followed by the addition of Hünigs base (DIPEA, 43 mg, 0.333 mmol) and the acid chloride 10 (2,3-difluoro-4-methyl-benzylchloride, 21.2 mg, 0.111 mmol). The reaction mixture was shaken at rt for 12 h. The solvent was evaporated and the residue dissolved in acetonitrile/formic acid=1/1 (1 ml) and purified by preparative HPLC to give 35 mg (52%) of 11 as a white solid.

All final compounds prepared by parallel chemistry techniques were analyzed by LC-MS. 5% of the library-compounds were analyzed by ¹H-NMR. The precursors were analyzed by LC-MS, ¹H-NMR and occasionally by ¹³C-NMR.

### Synthesis of N-[1-(3-Methyl-butyl)-piperidin-4-yl]-4-pentyl-N-[4-(5-phenyl-[1,2,4]oxadiazol-3-yl)-benzyl]-benzamide

### EXAMPLE 127

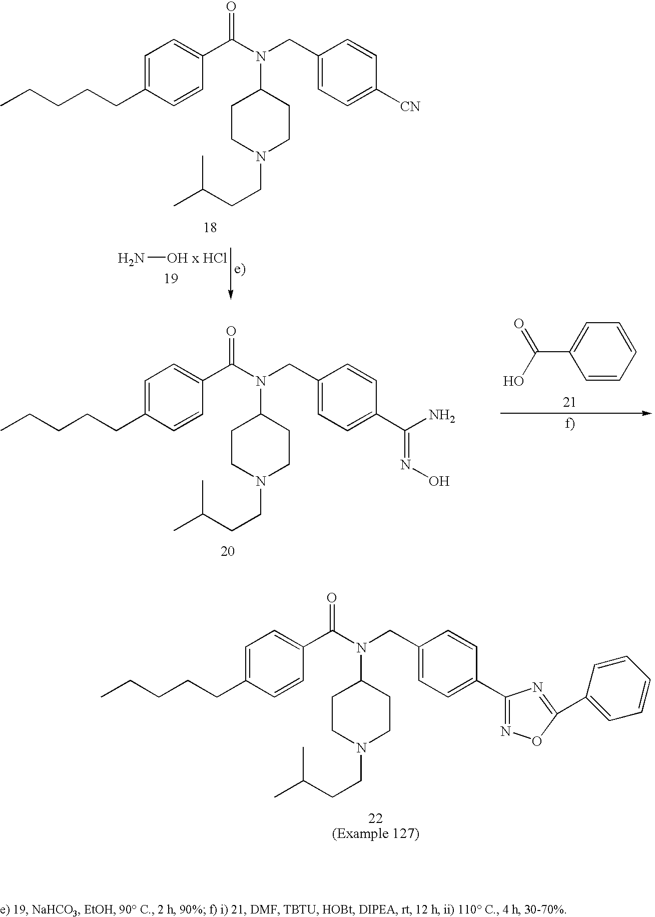

Compound 18 was prepared according to procedures described in the synthetic protocols for the preparation of example 2.

N-[4-(N-Hydroxycarbamimidoyl)-benzyl]-N-[1-(3-methyl-butyl)-piperidin-4-yl]-4-pentyl-benzamide (20): Compound 18 (9 g, 19.58 mmol) was dissolved in ethanol (40 ml) followed by the addition of hydroxylamine hydrochloride (1.5 g, 21.6 mmol) and sodium hydrogencarbonate (1.81 g, 21.6 mmol). The reaction mixture was heated to reflux for 1 h. The ethanol was removed under reduced pressure. The residue was taken up into water (100 ml) and extracted with ethyl acetate (3×60 ml). The combined organic layers were dried over magnesium sulfate, filtered and evaporated to give the product 20 (5.8 g, 60%), which was used in the subsequent parallel chemistry step without further purification.

N-[1-(3-Methyl-butyl)-piperidin-4-yl]-4-pentyl-N-[4-(5-phenyl-[1,2,4]oxadiazol-3-yl)-benzyl]-benzamide (22): Benzoic acid (21, 24.4 mg, 0.2 mmol) was dissolved in DMF (1 ml) and TBTU (64.2 mg, 0.2 mmol), HOBt (5.4 mg, 0.04 mmol) and DIPEA (129 mg, 1 mmol) were added. The reaction mixture was stirred for 5 min followed by the addition of compound 20 (98.5 mg, 0.2 mmol) dissolved in DMF (3 ml). Stirring was continued for 14 h. The reaction mixture was then heated to 110° C. for 4 h and subsequently poured onto ice/water (20 ml). The product was extracted with ethyl acetate (3×15 ml). The combined organic layers were washed with brine, dried over magnesium sulfate, filtered and the solvent was evaporated under reduced pressure. The crude product was purified by preparative HPLC to give compound 22 (13 mg, 11%).

### Synthesis of N-[1-(3-Methyl-butyl)-piperidin-4-yl]-N-[4-(3-methyl-isoxazol-5-yl)-benzyl]-4-pentyl-benzamide

### EXAMPLE 144

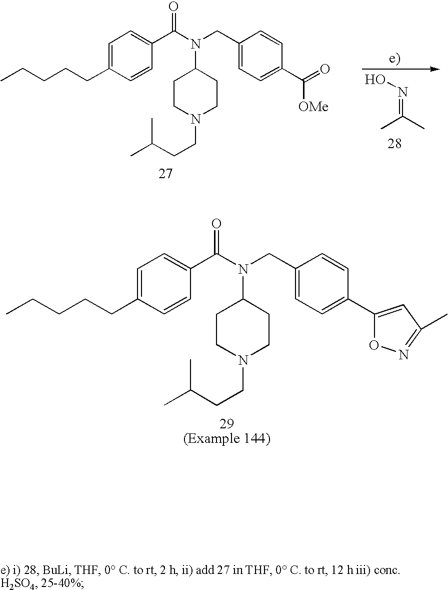

Compound 27 (4-{[[1-(3-methyl-butyl)-piperidin-4-yl]-(4-pentyl-benzoyl)-amino]-methyl}-benzoic acid methyl ester) was prepared according to procedures described above and served as the starting material for compound 29 and analoguous derivatives in a parallel chemistry setting.

N-[1-(3-Methyl-butyl)-piperidin-4-yl]-N-[4-(3-methyl-isoxazol-5-yl)-benzyl]-4-pentyl-benzamide (29): Acetone oxime (28, 44 mg, 0.6 mmol)) was dissolved in THF (1.2 ml) and cooled to 0° C. BuLi (0.83 ml of 1.6M solution in hexane) was added and the reaction mixture was allowed to warm to rt for 1 h followed by the addition of a solution of compound 27 (98.5 mg, 0.2 mmol) in THF (2 ml) at 0° C. The reaction mixture was stirred at rt for 14 h followed by slow addition of conc. sulfuric acid (0.08 ml) and stirring was continued for 15 min. The mixture was then poured onto sat. sodium carbonate solution (4 ml) and the product extracted with ethyl acetate (3×4 ml). The combined organic layers were dried over magnesium sulfate, filtered and the solvent was evaporated. The residue was purified by preparative HPLC to give compound 29 (15.4 mg, 15%).

### Synthesis of N-(4-Cyclopentanecarbonyl-benzyl)-N-[1-(3-methyl-butyl)-piperidin-4-yl]-4-pentyl-benzamide

### EXAMPLE 70

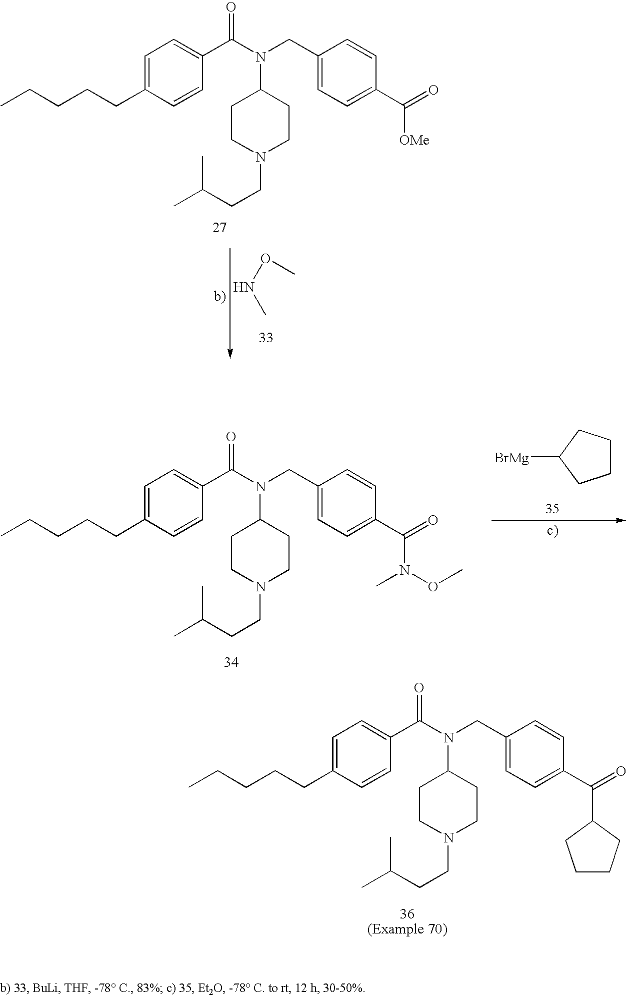

The precursor 27 was prepared according to procedures described above.

Compound 34: N,O-Dimethylhydroxylamine hydrochloride (4.03 g, 41.28 mmol) was dissolved in THF 67 ml and cooled to −78° C. followed by the addition of BuLi (51.5 ml 1.6M solution in hexane, 82.53 mmol). The reaction mixture was stirred for 15 min without cooling, then cooled again to −78° C. followed by the addition of a solution of 27 (2.26 g, 4.59 mmol) in THF (25 ml). The resulting reaction mixture was stirred for an additional 90 min at −78° C. and then quenched at that temperature by the addition of sat. ammonium chloride solution (250 ml). The product was extracted with ethyl acetate (3×100 ml). The combined organic layers were dried over magnesium sulfate, filtered and the solvent was evaporated under reduced pressure. The crude residue was purified by flash chromatography (silica gel; DCM/MeOH=9.25/0.75) to give intermediate 34 (4.0 g, 83%) as yellow oil.

N-(4-Cyclopentanecarbonyl-benzyl)-N-[1-(3-methyl-butyl)-piperidin-4-yl]-4-pentyl-benzamide (36, Example 70): Example 70 was prepared in a parallel chemistry setting. Intermediate 34 (50 mg, 0.096 mmol) was dissolved in diethylether (0.5 ml) and cooled to −78° C. followed by the addition of cyclopentyl magnesium bromide (35, excess). Stirring at −78° C. was continued for 30 min. The reaction mixture was then allowed to warm to rt and stirring was continued for 12 h, methanol (1 ml) was added and the mixture was filtered in order to remove the precipitate. The solvents were evaporated under reduced pressure and the residue was purified by preparative HPLC to give compound 36 (14 mg, 27%).

### Synthesis of 5-Pentyl-thiophene-2-carboxylic acid (45)

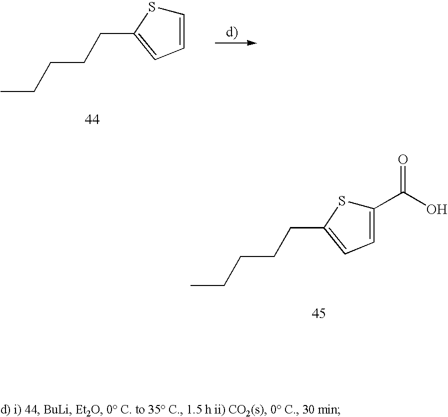

BuLi (1.6M in hexane, 20.3 ml, 32.41 mmol) was cooled to 0° C. under an atmosphere of nitrogen. 2-pentylthiophene (44) (5.0 g, 32.41 mmol), dissolved in diethylether (9.5 ml) was slowly added and stirring continued for 20 min. The reaction mixture was then refluxed for 1 h and cooled again to 0° C. The reaction mixture was poured onto a mixture of solid CO₂ in diethylether at 0° C. and stirring continued for 30 min followed by the addition of water and further stirring for 1 h. The pH was then adjusted to 2 by the addition of conc. HCl. The product was extracted with ethyl acetate (3×60 ml). The combined organic layers were dried over magnesium sulfate, filtered and the solvent was evaporated under reduced pressure. The residue was purified by flash chromatography (silica gel, heptane/ethyl acetate=1/1) to give compound 45 (4.99 g, 88%).

### Synthesis of 4-[(4′-Methoxycarbonyl-biphenyl-4-ylmethyl)-(5-pentyl-thiophene-2-carbonyl)-amino]-piperidine-1-carboxylic acid tert-butyl ester (46)

(represents a general method for the acylation of amines with heteroaryl-carboxylic acids)

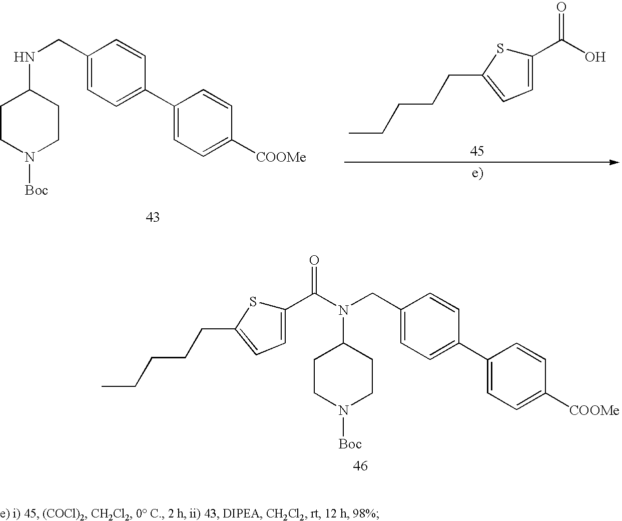

Compound 43 (1.87 g, 9.422 mmol) was dissolved in diethylether (50 ml) and cooled to 0° C. followed by the addition of oxalylchloride (5.02 ml, 59.36 mmol) and 5 drops of DMF. The reaction mixture was stirred at 0° C. for 3 h. The solvent was evaporated under reduced pressure at rt, CCl₄ (20 ml) was added and evaporated again under reduced pressure. This procedure was repeated 3 times in order to fully remove the oxalylchloride. The residue was dissolved in DCM (50 ml) followed by the addition of DIPEA (4.84 ml, 28.26 mmol) and compound 45 (4.0 g, 9.42 mmol). The reaction mixture was stirred at rt for 12 h, then poured onto a mixture of 2M HCl/DCM. The organic layer was separated, the aqueous phase extracted with DCM (2×), the combined organic layers were washed with sat. sodium hydrogencarbonate solution and brine, dried over magnesium sulfate, filtered and the solvent was removed under reduced pressure. The residue was purified by flash chromatography (silica gel, DCM/methanol=9.75/0.25) to give compound 46 (5.78 g, quant.).

### Synthesis of 4-[(4′-Methoxycarbonyl-biphenyl-4-ylmethyl)-(4-pentyl-thiophene-2-carbonyl)-amino]-piperidine-1-carboxylic acid tert-butyl ester (51)

(represents a general method for the Sonogashira reaction onto heteroaryl bromides and the subsequent reduction of the triple bond to the single bond)

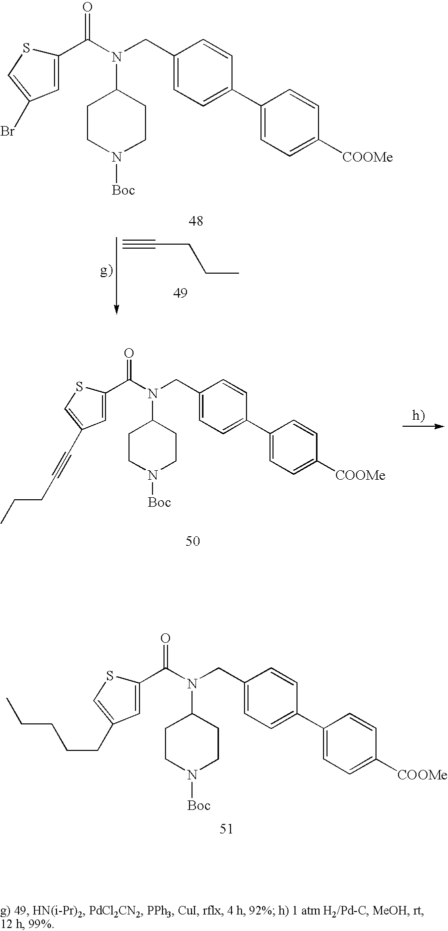

Compound 48 (4.38 g, 7.14 mmol) was dissolved in diisopropylamine (12 ml). Dichloro-bis-(benzonitrile)palladium (1.1 g, 2.86 mmol), triphenylphosphine (1.5 g, 2.86 mmol) and copper iodide (0.544 g, 2.86 mmol) were added and the mixture was degassed with nitrogen for 10 min followed by the addition of pentyne (49) (1.4 ml, 14.277 mmol). The reaction mixture is refluxed for 4 h, then evaporated to dryness. The residue is purified by flash chromatography (silica gel, ethyl acetate/heptane=3/7) to give compound 50 (3.96 g, 92%).

Compound 50 (2.04 g, 3.4 mmol) was dissolved in methanol (40 ml) and placed in an atmosphere of nitrogen. The catalyst Pd—C (10% on charcoal, 0.361 g) was added and the reaction mixture was stirred at rt for 40 h under an atmosphere of hydrogen (1 atm). The mixture was filtered over celite, the solvent evaporated under reduced pressure and the residue purified by flash chromatography (silica gel, ethyl acetate/heptane=4/6) to give product 51 (1.69 g, 83%).

The precursor 55 was prepared according to the same sequence of reactions with the appropriate starting materials.

### Synthesis of 5-Pentyl-pyridine-2-carboxylic acid (67)

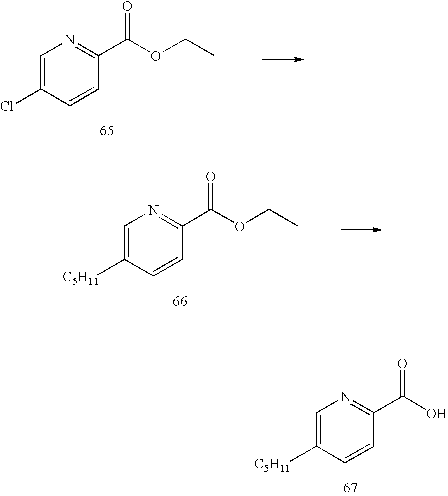

Compound 66: To a solution of pentylmagnesiumbromide (1 M in THF, 64.6 ml, 64.6 mmol) was added a solution of zinc chloride (1 M in THF, 70.5 ml, 70.5 mmol) at rt and stirring was continued for 15 min followed by the addition of bis-triphenylphosphine palladium(II) dichloride (1.32 g, 1.84 mmol) and 5-chloropyridine-2-carboxylic acid ethyl ester (6 g, 32.3 mmol). Stirring was continued at rt overnight. Hydrochloride acid (1 M aq) was added to pH=4. The product was extracted with DCM (3×220 ml). The combined organic layers were dried over magnesium sulfate, filtered and the solvent was evaporated under reduced pressure. The residue was purified by column chromatography (silica gel, heptane/EtOAc=3/1) to give 2.02 g (28%) of 5-pentyl-pyridine-2-carboxylic acid ethylester (66). LC-MS: t_{R}=0.95 min; [M+H]⁺=222.25.

Compound 67: The ethylester 66 (7.37 g, 33.3 mmol) was dissolved in MeOH (150 ml) followed by the addition of aq. NaOH (2 M, 53 ml, 106 mmol). The reaction mixture was stirred at rt for 2.5 h and concentrated under reduced pressure. To the aqueous residue was added EtOAc (80 ml) followed by the extraction with NaOH (10%, 2×80 ml). The combined aqueous layers were acidified to pH=5 by the addition of hydrochloride acid (1 M aq). The product was extracted with EtOAc (2×250 ml). The combined organic layers were dried with magnesium sulfate, filtered and concentrated under reduced pressure to give 5.53 g (85%) of 5-pentyl-pyridine-2-carboxylic acid (67). LC-MS: t_{R}=0.66 min; [M+H]⁺=194.16.

### Synthesis of 4-(4-Bromo-benzylamino)-piperidine-1-carboxylic acid tert-butyl ester (69)

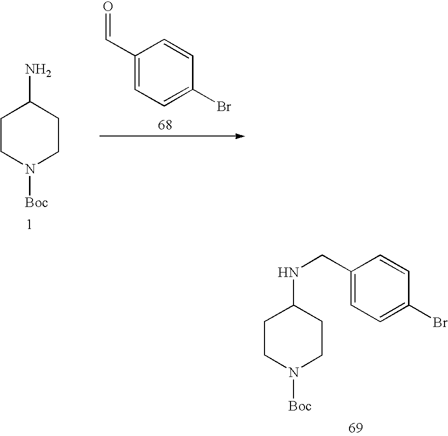

According to the typical procedure A): From 4-bromobenzaldehyde (68) (3.9 g, 0.021 mol) and 1-Boc-4-amino-piperidine (5 g, 0.021 mol) was obtained 7.23 g (92%) of 4-(4-bromo-benzylamino)-piperidine-1-carboxylic acid tert-butyl ester (69). LC-MS: t_{R}=0.78 min; [M+H]⁺=370.28.

### Synthesis of 4-[(4-Bromo-benzyl)-(5-pentyl-pyridine-2-carbonyl)-amino]-piperidine-1-carboxylic acid tert-butyl ester (70)

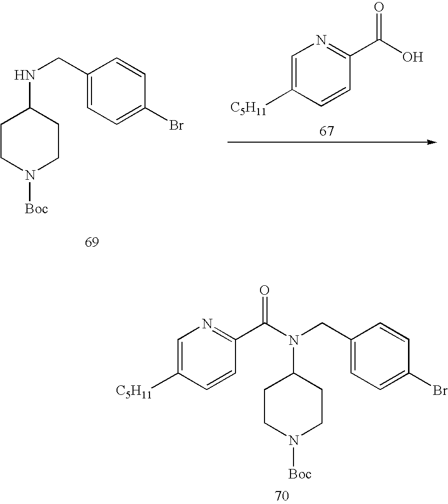

The carboxylic acid 67 (575 mg, 2.97 mmol) was dissolved in DCM (45 ml) followed by the addition of TBTU (956 mg, 2.98 mmol) and DIPEA (1.05 g, 8.12 mmol). Stirring was continued at rt for 5 min followed by the addition of the amine 69 (1 g, 2.7 mmol). Stirring was continued at rt for 90 min. The organic solvent was removed under reduced pressure, and water was added (60 ml) followed by extraction with EtOAc (3×60 ml). The combined organic layers were washed with brine (2×70 ml) dried over magnesium sulfate, filtered and concentrated under reduced pressure. The residue was purified by column chromatography (silica gel, EtOAc/heptane=1/1) to give 1.27 g (86%) of 4-[(4-bromo-benzyl)-(5-pentyl-pyridine-2-carbonyl)-amino]-piperidine-1-carboxylic acid tert-butyl ester (70). LC-MS: t_{R}=1.13 min; [M+H]⁺=544.44.

### Synthesis of 5-Pentyl-pyridine-2-carboxylic acid (4-bromo-benzyl)-piperidin-4-yl-amide trifluoroacetate (71)

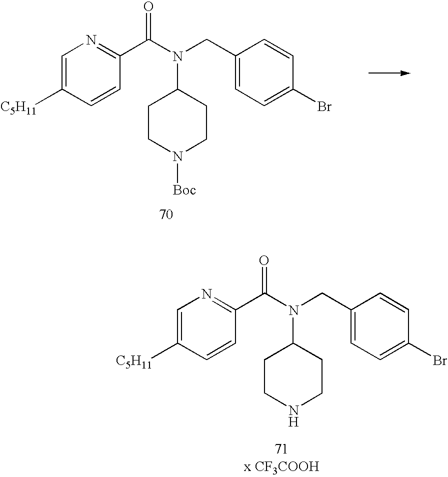

Compound 70 (1.27 g, 2.33 mmol) was dissolved in DCM (20 ml) and cooled to 0° C. followed by slow addition of TFA (3.9 g, 34.9 mmol). The reaction mixture was stirred at 0° C. for 2 h and at rt for 1 h. The solvent was removed under reduced pressure and the product was dried at HV to give 1.29 g (quantitative yield) of 5-pentyl-pyridine-2-carboxylic acid (4-bromo-benzyl)-piperidin-4-yl-amide trifluoroacetate (71). LC-MS: t_{R}=0.84 min; [M+H]⁺=446.35.

### Synthesis of 5-Pentyl-pyridine-2-carboxylic acid (4-bromo-benzyl)-[1-(3-methylbutyl)-piperidin-4-yl]-amide (72)

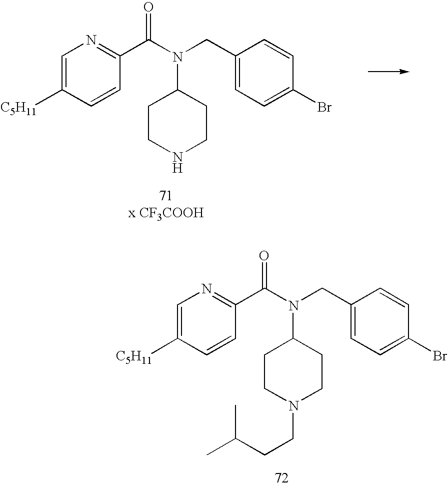

According to the typical procedure D): Compound 71 (1.03 g, 2.33 mmol) was transformed into 5-pentyl-pyridine-2-carboxylic acid (4-bromo-benzyl)-[1-(3-methylbutyl)-piperidin-4-yl]-amide (72) (1.2 g; quantitative yield). LC-MS: t_{R}=0.96 min; [M+H]⁺=516.48.

### Synthesis of 5-Pentyl-pyridine-2-carboxylic acid (3′-methyl-biphenyl-4-ylmethyl)-[1-(3-methyl-butyl)-piperidin-4-yl]-amide (74)

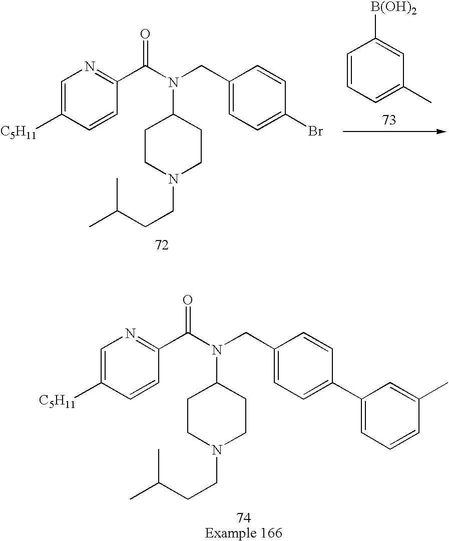

The arylbromide 72 (100 mg, 0.194 mmol) was dissolved in a mixture of i-propanol/toluene (1/1, 2 ml) followed by the addition of an aqueous solution of potassium carbonate (2 M, 0.5 ml) and the boronic acid 73 (31 mg, 0.21 mmol). The mixture was degassed with argon for 5 min then heated to 85° C. and tetrakistriphenylphosphine palladium (6.7 mg, 0.006 mmol) was added. Heating was continued for 2 h followed by cooling to rt, the addition of water (2 ml) and extraction with EtOAc (3×1.5 ml). The combined organic layers were dried over magnesium sulfate, concentrated under reduced pressure and the residue was purified by preparative HPLC to give 62.2 mg (61%) of 5-pentyl-pyridine-2-carboxylic acid (3′-methyl-biphenyl-4-ylmethyl)-[1-(3-methyl-butyl)-piperidin-4-yl]-amide (74, Example 166). LC-MS: t_{R}=0.98 min; [M+H]⁺=526.24.

Examples 165 to 183 were prepared according to this procedure.

### Synthesis of 5-Pentyl-pyridine-2-carboxylic acid [1-(3-methyl-butyl)-piperidin-4-yl]-[4-(3,4,5,6-tetrahydro-2H-[4,4′]bipyridinyl-1-yl)-benzyl]-amide (76)

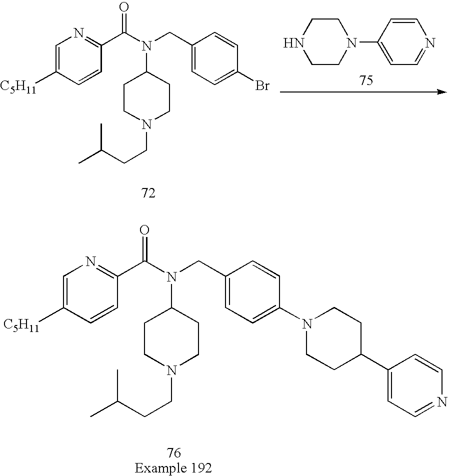

The arylbromide 72 (45 mg, 0.087 mmol) was dissolved in dioxane (1 ml) followed by the addition of sodium tert.-butoxide (12 mg, 0.122 mmol) and the piperazine derivative 75 (17.66 mg, 0.105 mmol). The mixture was degassed with argon and heated to 110° C. followed by the addition of the catalyst SK-CC02-A (1 mg, 0.002 mol). The mixture was stirred at 110° C. for 30 min, cooled to rt, water (2 ml) was added followed by extraction with EtOAc (3×1 ml), and the combined organic layers were dried over magnesium sulfate, filtered and concentrated under reduced pressure. The residue was purified by preparative HPLC to give 27 mg (52%) 5-pentyl-pyridine-2-carboxylic acid [1-(3-methyl-butyl)-piperidin-4-yl]-[4-(3,4,5,6-tetrahydro-2H-[4,4′]bipyridinyl-1-yl)-benzyl]-amide (76, Example 192). LC-MS: t_{R}=0.76 min; [M+H]⁺=597.28.

Examples 184 to 193 were prepared according to this procedure.

### Synthesis of 4-[(4-Methoxycarbonyl-benzyl)-(5-pentyl-pyridine-2-carbonyl)amino]-piperidine-1-carboxylic acid tert-butyl ester (77)

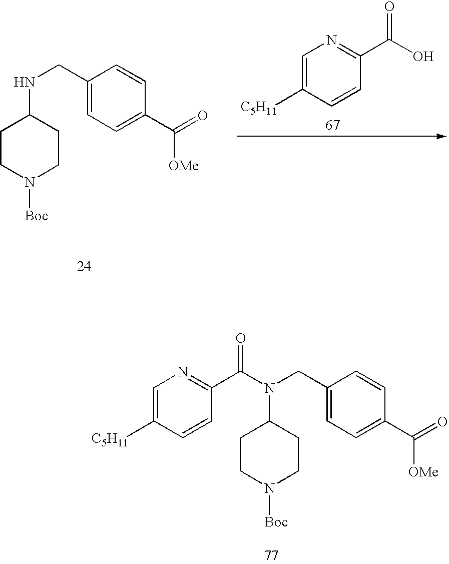

According to the procedure described for the preparation of compound 70, amine 24 (1.5 g, 4.3 mmol) was reacted with acid 67 (915 mg, 4.73 mmol) to give 1.93 g (86%) of 4-[(4-methoxycarbonyl-benzyl)-(5-pentyl-pyridine-2-carbonyl)-amino]-piperidine-1-carboxylic acid tert-butyl ester (77). LC-MS: t_{R}=1.09 min; [M+H]⁺=524.3.

### Synthesis of 4-{[(5-Pentyl-pyridine-2-carbonyl)-piperidin-4-yl-amino]-methyl}-benzoic acid methyl ester trifluoroacetate (78)

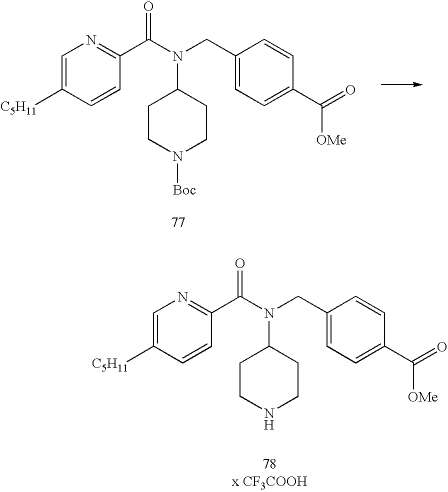

According to the procedure described for the preparation of compound 71, the derivative 77 (1.93 g, 3.69 mmol) was Boc-deprotected to give 1.95 g (quantitative yield) of 4-{[(5-pentyl-pyridine-2-carbonyl)-piperidin-4-yl-amino]-methyl}-benzoic acid methyl ester trifluoroacetate (78). LC-MS: t_{R}=0.80 min; [M+H]⁺=424.39.

### Synthesis of 4-{[[1-(3-Methyl-butyl)-piperidin-4-yl]-(5-pentyl-pyridine-2-carbonyl)-amino]-methyl}-benzoic acid methyl ester (79)

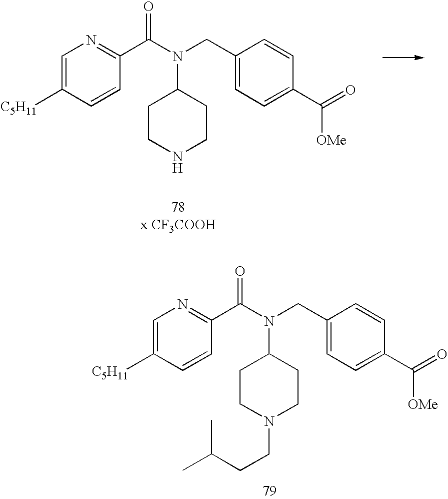

According to procedure described for the preparation of compound 72, the precursor 78 (1.56 g, 3.69 mmol) was reacted with isovaleraldehyde (318 mg, 3.69 mmol) to give 1.81 g (quantitative yield) of 4-{[[1-(3-methyl-butyl)-piperidin-4-yl]-(5-pentyl-pyridine-2-carbonyl)-amino]-methyl}-benzoic acid methyl ester (79). LC-MS: t_{R}=0.93 min; [M+H]⁺=494.54.

### Synthesis of 4-{[[1-(3-Methyl-butyl)-piperidin-4-yl]-(5-pentyl-pyridine-2-carbonyl)-amino]-methyl}-benzoic acid (80)

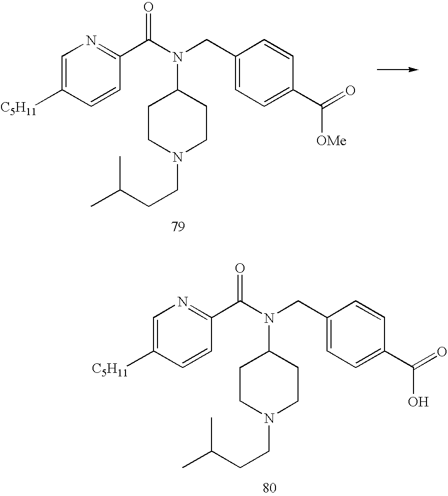

Methylester 79 (1.17 g, 2.39 mmol) was dissolved in methanol (30 ml) followed by the addition of lithiumhydroxide solution (2M, 4.83 ml) and stirring of the reaction mixture at rt for 14 h. Citric acid solution (10%) was added to adjust the pH of the mixture to 5 and the methanol was evaporated under reduced pressure. The remaining aqueous layer was extracted with EtOAc (2×60 ml). The combined organic layers were dried over magnesium sulfate, filtered and concentrated under reduced pressure to give 1.03 g (89%) of Synthesis of 4-{[[1-(3-methyl-butyl)-piperidin-4-yl]-(5-pentyl-pyridine-2-carbonyl)-amino]-methyl}-benzoic acid (80) as a white powder. LC-MS: t_{R}=0.82 min; [M+H]⁺=480.38.

### Synthesis of 5-Pentyl-pyridine-2-carboxylic acid [4-(2,6-difluoro-benzylcarbamoyl)-benzyl]-[1-(3-methyl-butyl)-piperidin-4-yl]-amide (81)

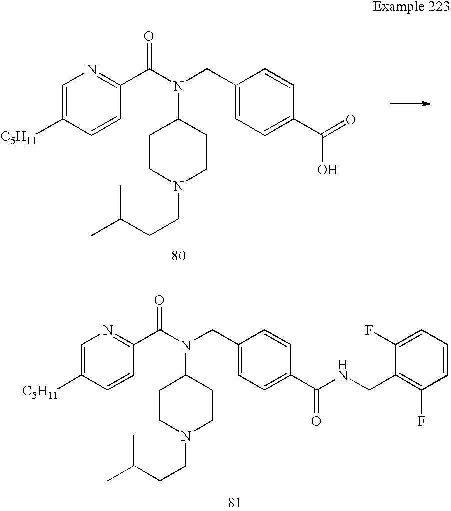

The acid 80 (50 mg, 0.104 mmol) was dissolved in acetonitirle (1 ml) followed by the addition of TBTU (36.8 mg, 0.115 mmol) and DIPEA (40.49 mg, 0.313 mmol). Stirring was continued for 5 min. 2,6-Difluoro-benzylamine (17 mg, 0.115 mmol) was added and stirring was continued for 16 h. The reaction mixture was filtered and directly purified by preparative HPLC to give 31.5 mg (50%) of 5-pentyl-pyridine-2-carboxylic acid [4-(2,6-difluoro-benzylcarbamoyl)-benzyl]-[1-(3-methyl-butyl)piperidin-4-yl]-amide (81, Example 223). LC-MS: t_{R}=0.94 min; [M+H]⁺=605.54.

Examples 199 to 209 and 222 to 225 were prepared according to this procedure.

### Synthesis of 4-[(4-Cyano-benzyl)-(5-pentyl-pyridine-2-carbonyl)-amino]-piperidine-1-carboxylic acid tert-butyl ester (82)

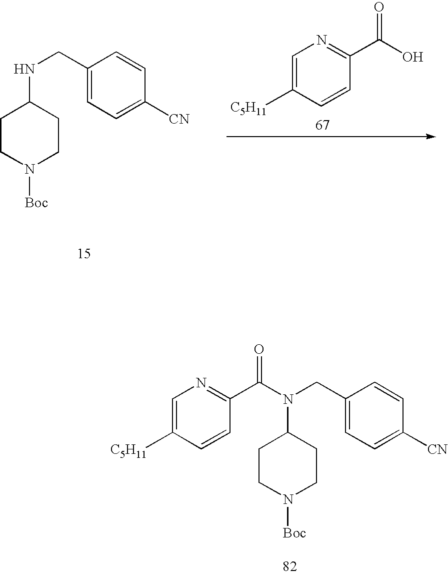

According to the procedure described for the preparation of compound 77, derivative 15 (200 mg, 0.634 mmol) was reacted with acid 67 (135 mg, 0.698 mmol) to give 263 mg (84%) of 4-[(4-cyano-benzyl)-(5-pentyl-pyridine-2-carbonyl)-amino]-piperidine-1-carboxylic acid tert-butyl ester (82). LC-MS: t_{R}=1.09 min; [M+H]⁺=491.65.

### Synthesis of 5-Pentyl-pyridine-2-carboxylic acid (4-cyano-benzyl)-piperidin-4-yl-amide trifluoroacetate (83)

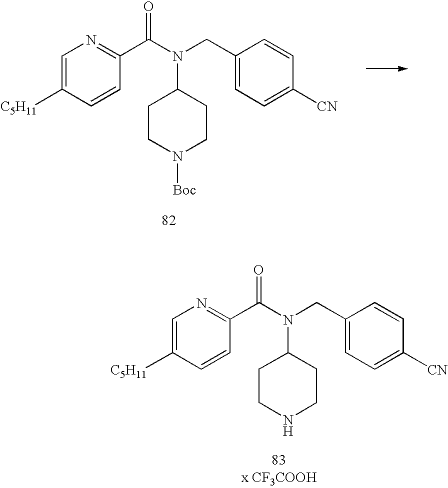

According to the procedure described for the reparation of compound 78, derivative 82 (2.0 g, 4.08 mmol) was deprotected to give 2 g (quantitative yield) of synthesis of 5-pentyl-pyridine-2-carboxylic acid (4-cyano-benzyl)-piperidin-4-yl-amide trifluoroacetate (83). LC-MS: t_{R}=0.81 min; [M+H]⁺=391.34.

### Synthesis of 5-Pentyl-pyridine-2-carboxylic acid (4-cyano-benzyl)-[1-(3-methylbutyl)-piperidin-4-yl]-amide (84)

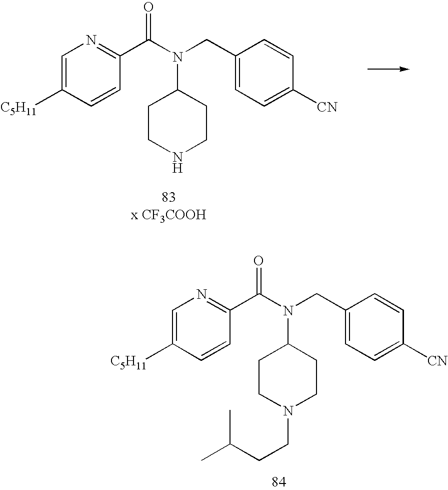

According to procedure described for the preparation of compound 72, the precursor 83 (1.595 g, 4.084 mmol) was reacted with isovaleraldehyde (351 mg, 4.084 mmol) to give 1.88 g (quantitative yield) of 5-pentyl-pyridine-2-carboxylic acid (4-cyanobenzyl)-[1-(3-methyl-butyl)-piperidin-4-yl]-amide (84). LC-MS: t_{R}=0.91 min; [M+H]⁺=461.51.

### Synthesis of 5-Pentyl-pyridine-2-carboxylic acid [4-(N-hydroxy-carbamimidoyl)benzyl]-[1-(3-methyl-butyl)-piperidin-4-yl]-amide (85)

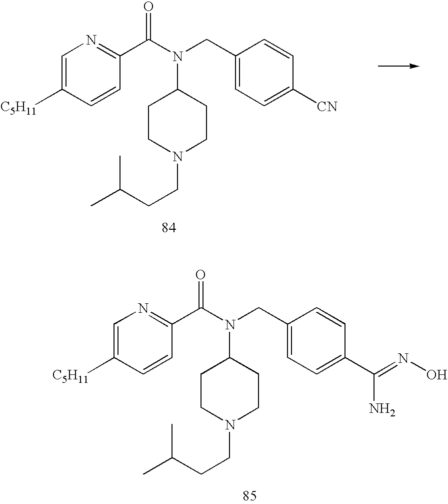

The nitrile 84 (2.17 g, 4.71 mmol) was dissolved in ethanol (40 ml) followed by the addition of hydroxylamine hydrochloride (1.14 g, 16.48 mmol) and sodium hydrogen carbonate (1.38 g, 16.48 mmol). The reaction mixture was refluxed for 16 h. The ethanol was evaporated under reduced pressure and water (30 ml) was added. The product was extracted with EtOAc (5×40 ml). The combined organic layers were washed with brine (70 ml), dried over magnesium sulfate, filtered and concentrated under reduced pressure. The residue was purified by column chromatography (silica gel, methanol/DCM=1/9) to give 1.05 g (45.2%) of 5-pentyl-pyridine-2-carboxylic acid [4-(N-hydroxy-carbamimidoyl)-benzyl]-[1-(3-methyl-butyl)-piperidin-4-yl]-amide (85). LC-MS: t_{R}=0.73 min; [M+H]⁺=494.50.

### Synthesis of 5-Pentyl-pyridine-2-carboxylic acid [1-(3-methyl-butyl)-piperidin-4-yl]-[4-(5-pyridin-3-yl-[1,2,4]oxadiazol-3-yl)-benzyl]-amide (87)

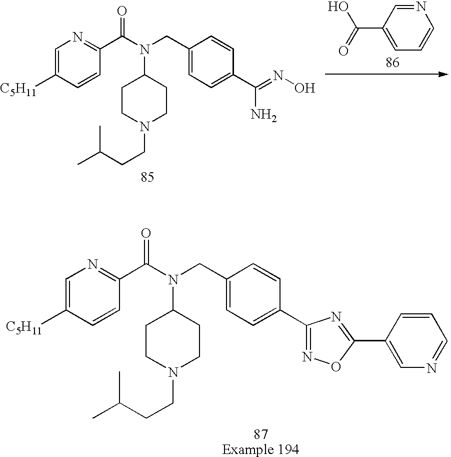

3-Pyridyl carboxylic acid (86) (18.7 mg, 0.152 mmol) was dissolved in DMF (1 ml). TBTU (65 mg, 0.203 mmol), HOBt (3.1 mg, 0.02 mmol) and DIPEA (39 mg, 0.304 mmol) were added and stirring continued for 10 min followed by the addition of compound 85 (50 mg, 0.101 mmol). Stirring was continued for 16 h at rt followed by heating the reaction mixture to 90° C. for 1 h. Water (2 ml) was added and the product was extracted with EtOAc (3×2 ml). The combined organic layers were dried over magnesium sulfate, filtered and concentrated in vacuo. The crude material was purified by preparative HPLC to give 34 mg (58%) of 5-pentyl-pyridine-2-carboxylic acid [1-(3-methyl-butyl)-piperidin-4-yl]-[4-(5-pyridin-3-yl-[1,2,4]oxadiazol-3-yl)benzyl]-amide (87, Example 194). LC-MS: t_{R}=0.97 min; [M+H]⁺=581.59.

Examples 194 to 198 were prepared according to this procedure.

### Synthesis of [1-(3-Methyl-butyl)-piperidin-4-yl]-carbamic acid tert-butyl ester (91)

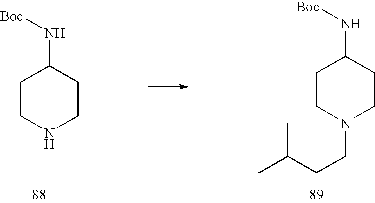

According to the typical procedure D), compound 88 (10 g, 50 mmol) was reacted with isovaleraldehyde (4.3 g, 50 mmol) to give 12.39 g (92%) of [1-(3-methyl-butyl)piperidin-4-yl]-carbamic acid tert-butyl ester (91). LC-MS: t_{R}=0.70 min; [M+H]⁺=271.32.

### Synthesis of 1-(3-Methyl-butyl)-piperidin-4-ylamine hydrochloride (90)

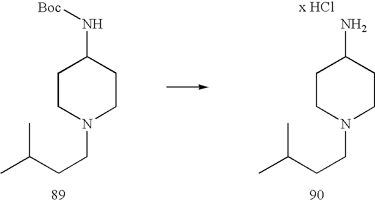

Compound 89 (12 g, 44.4 mmol) was dissolved in DCM (130 ml) and cooled to 0° C. followed by the addition of HCl in dioxane (4M, 130 ml). The reaction mixture was stirred at 0° C. for 90 min then concentrated under reduced pressure to give 9.61 g (82%) of 1-(3-methyl-butyl)-piperidin-4-ylamine hydrochloride (90) as a white solid.

LC-MS: t_{R}=0.21 min; [M+H]⁺=no peak detected.

### Synthesis of [5-(3,4-Dichloro-phenyl)-furan-2-ylmethyl]-[1-(3-methyl-butyl)-piperidin-4-yl]-amine (92)

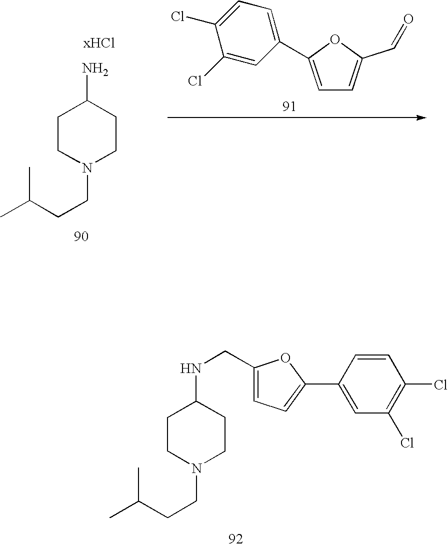

According to the typical procedure A), compound 90 (500 mg, 2.06 mmol) was reacted with aldehyde 91 (511 mg, 2.06 mmol) to give 813 mg (quantitative yield) of [5-(3,4-dichloro-phenyl)-furan-2-ylmethyl]-[1-(3-methyl-butyl)-piperidin-4-yl]-amine (92). LC-MS: t_{R}=0.73 min; [M+H]⁺=395.35.

### Synthesis of N-[5-(3,4-Dichloro-phenyl)-furan-2-ylmethyl]-N-[1-(3-methyl-butyl)piperidin-4-yl]-4-pentyl-benzamide (93)

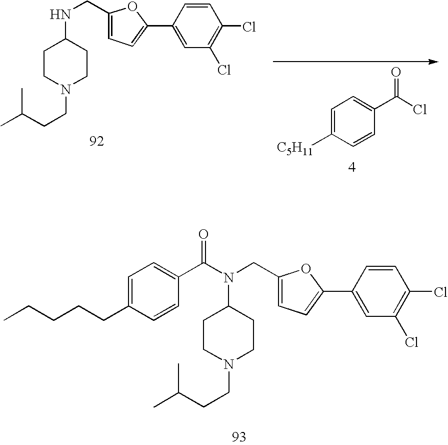

According to the typical procedure B), compound (92) (82.56 mg, 0.209 mmol) was reacted with the acid chloride 4 (40 mg, 0.19 mmol) to give 73.5 mg (68%) of N-[5-(3,4-dichloro-phenyl)-furan-2-ylmethyl]-N-[1-(3-methyl-butyl)-piperidin-4-yl]-4-pentyl-benzamide (93, Example 220). LC-MS: t_{R}=1.03 min; [M+H]⁺=569.19.

Examples 210 to 221 were prepared according to this procedure.

### Synthesis of 4-{[[1-(3-Methyl-butyl)-piperidin-4-yl]-(4-pentyl-benzoyl)-amino]-methyl}-benzoic acid amide (94)

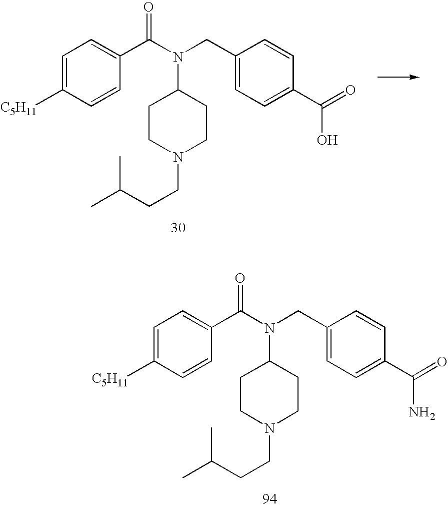

Compound 30 (2.5 g, 5.26 mmol) was dissolved in DMF (100 ml) and PyBOP (3.01 g, 5.78 mmol) was added. The mixture was stirred at rt for 5 min, cooled to 0° C. followed by the addition of DIPEA (2.92 g, 22.6 mmol) and ammonium chloride (563 mg, 10.52 mmol). The reaction mixture was then stirred at rt for 2 h, water was added (100 ml) and the product was extracted with EtOAc (3×100 ml). The combined organic layers were washed with brine (100 ml), dried over magnesium sulfate, filtered and the solvent was evaporated under reduced pressure. The residue was purified by column chromatography (silica gel, methanol/DCM=1/9) to give 2.6 g (quantitative yield) of 4-{[[1-(3-methyl-butyl)-piperidin-4-yl]-(4-pentylbenzoyl)-amino]-methyl}-benzoic acid amide (94) as a white solid. LC-MS: t_{R}=0.88 min; [M+H]⁺=478.47.

### Synthesis of N-[1-(3-Methyl-butyl)-piperidin-4-yl]-4-pentyl-N-(4-thio-carbamoylbenzyl)-benzamide (95)

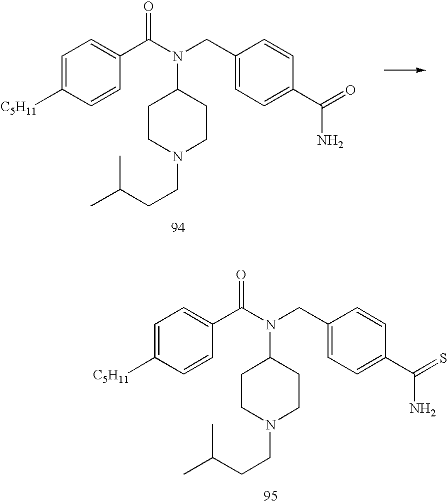

The primary amide derivative 94 (2.51 g, 5.26 mmol) was dissolved in dry THF (50 ml), cooled to 0° C. followed by the addition of Lawesson's reagent (1.06 g, 2.63 mmol). The reaction mixture was stirred at 0° C. for 2 h and at rt for 16 h, then concentrated under reduced pressure and the residue was purified by column chromatography (silica gel, methanol/DCM=5/95) to give 1.29 g (49%) of N-[1-(3-methyl-butyl)-piperidin-4-yl]-4-pentyl-N-(4-thio-carbamoyl-benzyl)-benzamide (95) as a green solid. LC-MS: t_{R}=0.91 min; [M+H]⁺=494.5.

### Synthesis of N-[1-(3-Methyl-butyl)-piperidin-4-yl]-4-pentyl-N-[4-(4-trifluoromethyl-thiazol-2-yl)-benzyl]-benzamide (96)

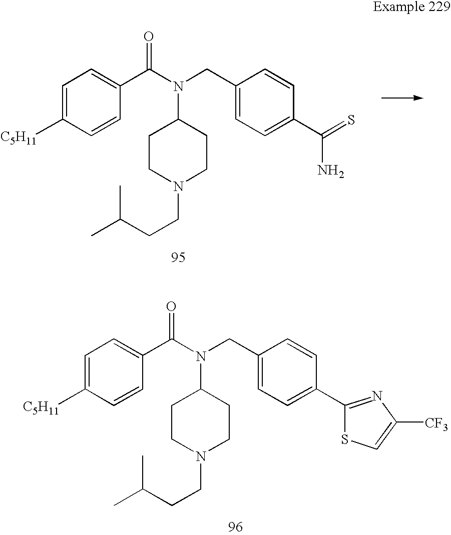

The thioamide 95 (50 mg, 0.101 mmol) was dissolved in 1,2-dimethoxyethane (1 ml) and potassium hydrogen carbonate (81 mg, 0.81 mmol) was added and stirring at rt continued for 10 min followed by the addition of 3-bromo-1,1,1-trifluoroacetone (60 mg, 0.304 mmol) and stirring was continued for 30 min at rt. The reaction mixture was cooled to 0° C. and a preformed solution of 2,6-lutidine (86.8 mg, 0.81 mmol) and TFAA (85 mg, 0.405 mmol) in 1,2-dimethoxyethane (0.5 ml) was added. Stirring at 0° C. was continued for 75 min, HCl aq. (1 M, 1.5 ml) was added and the product was evaporated with DCM (3×2 ml). The combined organic layers were dried over magnesium sulfate, filtered and concentrated under reduced pressure. The residue was purified by preparative HPLC to give 31 mg (52%) of N-[1-(3-methyl-butyl)piperidin-4-yl]-4-pentyl-N-[4-(4-tri-fluoromethyl-thiazol-2-yl)-benzyl]-benzamide (96, Example 229). LC-MS: t_{R}=1.01 min; [M+H]⁺=586.41.

Examples 229 to 232 were prepared according to this procedure.

The final compounds mentioned as examples in the following part of the patent application could be prepared by a suitable combination of synthetic protocols described above or by application of literature procedures as cited further above.

TABLE 1

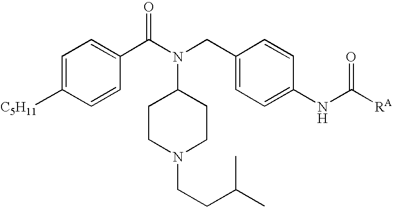

| Ex. No: | 1 | 2 | 3 |
|---|---|---|---|
| R^{A} | 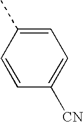 | 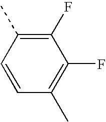 | 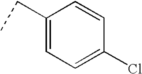 |
| LC-MS | 0.96 | 0.99 | 0.97 |
| t_{R} (min) | 579.96 | 604.11 | 602.54 |
| [M + H]⁺ | | | |
| Ex. No: | 4 | 5 | 6 |
| R^{A} | 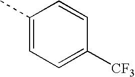 | 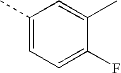 | 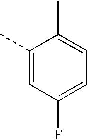 |
| LC-MS | 0.99 | 0.99 | 0.97 |
| t_{R} (min) | 622.10 | 586.12 | 586.13 |
| [M + H]⁺ | | | |
| Ex. No: | 7 | 8 | 9 |
| R^{A} | 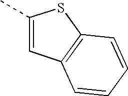 | 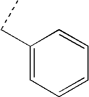 | 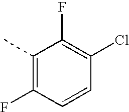 |
| LC-MS | 0.99 | 0.95 | 0.98 |
| t_{R} (min) | 610.55 | 568.55 | 624.06 |
| [M + H]⁺ | | | |
| Ex. No: | 10 | 11 | 12 |
| R^{A} | 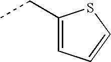 | 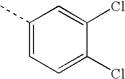 | 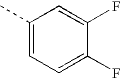 |
| LC-MS | 0.95 | 1.01 | 0.98 |
| t_{R} (min) | 574.84 | 622.03 | 590.09 |
| [M + H]⁺ | | | |
| Ex. No: | 13 | 14 | 15 |
| R^{A} | 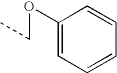 | 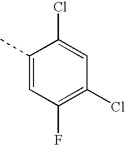 | 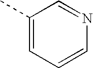 |
| LC-MS | 0.97 | 1.00 | 0.89 |
| t_{R} (min) | 584.60 | 640.05 | 555.31 |
| [M + H]⁺ | | | |
| Ex. No: | 16 | 17 | 18 |
| R^{A} | 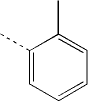 | 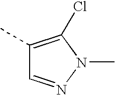 | 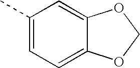 |
| LC-MS | 0.96 | 0.92 | 0.96 |
| t_{R} (min) | 568.13 | 592.53 | 598.09 |
| [M + H]⁺ | | | |
| Ex. No: | 19 | 20 | 21 |
| R^{A} | 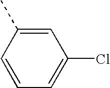 | 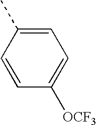 | 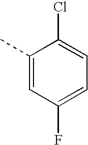 |
| LC-MS | 0.99 | 1.00 | 0.97 |
| t_{R} (min) | 588.06 | 638.11 | 606.05 |
| [M + H]⁺ | | | |
| Ex. No: | 22 | 23 | 24 |
| R^{A} | 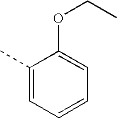 | 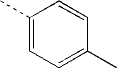 | 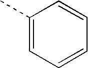 |
| LC-MS | 0.99 | 0.95 | 0.96 |
| t_{R} (min) | 598.14 | 568.58 | 554.30 |
| [M + H]⁺ | | | |
| Ex. No: | 25 | 26 | 27 |
| R^{A} | 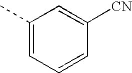 | 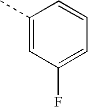 | 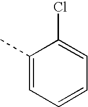 |
| LC-MS | 0.96 | 0.96 | 0.96 |
| t_{R} (min) | 579.12 | 572.56 | 588.07 |
| [M + H]⁺ | | | |
| Ex. No: | 28 | 29 | 30 |
| R^{A} | 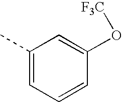 | 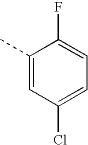 | 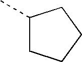 |
| LC-MS | 1.00 | 0.98 | 0.96 |
| t_{R} (min) | 638.10 | 606.41 | 546.45 |
| [M + H]⁺ | | | |
| Ex. No: | 31 | 32 | 33 |
| R^{A} | 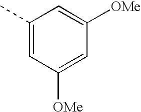 | 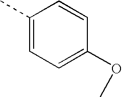 | 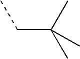 |
| LC-MS | 0.95 | 0.96 | 0.96 |
| t_{R} (min) | 614.13 | 584.32 | 548.58 |
| [M + H]⁺ | | | |
| Ex. No: | 34 | 35 | 36 |
| R^{A} | 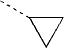 | 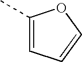 | 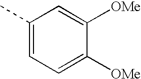 |
| LC-MS | 0.91 | 0.92 | 0.94 |
| t_{R} (min) | 518.56 | 544.50 | 614.12 |
| [M + H]⁺ | | | |
| Ex. No: | 37 | 38 | 39 |
| R^{A} | 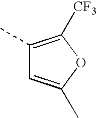 | 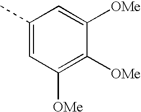 | 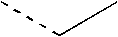 |
| LC-MS | 0.99 | 0.95 | 0.89 |
| t_{R} (min) | 626.54 | 644.65 | 506.54 |
| [M + H]⁺ | | | |
| Ex. No: | 40 | | |
| R^{A} | 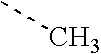 | | |
| LC-MS | 0.88 | | |
| t_{R} (min) | 492.52 | | |
| [M + H]⁺ | | | |

TABLE 2

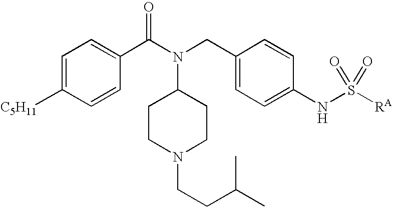

| Ex. No: | 41 | 42 | 43 |
|---|---|---|---|
| R^{A} | 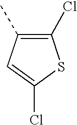 | 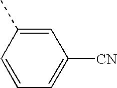 | 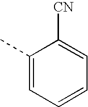 |
| LC-MS | 1.00 | 0.96 | 0.95 |
| t_{R} (min) | 664.48 | 615.47 | 615.52 |
| [M + H]⁺ | | | |
| Ex. No: | 44 | 45 | 46 |
| R^{A} | 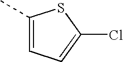 | 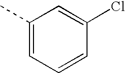 | 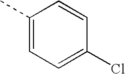 |
| LC-MS | 0.98 | 0.98 | 0.98 |
| t_{R} (min) | 630.43 | 624.52 | 624.49 |
| [M + H]⁺ | | | |
| Ex. No: | 47 | 48 | 49 |
| R^{A} | 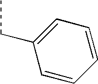 | 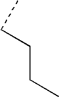 | 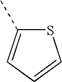 |
| LC-MS | 0.96 | 0.96 | 0.95 |
| t_{R} (min) | 604.51 | 570.53 | 596.44 |
| [M + H]⁺ | | | |
| Ex. No: | 50 | 51 | 52 |
| R^{A} | 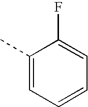 | 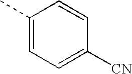 | 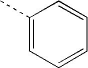 |
| LC-MS | 0.96 | 0.96 | 0.96 |
| t_{R} (min) | 608.52 | 615.49 | 590.51 |
| [M + H]⁺ | | | |
| Ex. No: | 53 | 54 | 55 |
| R^{A} | 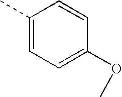 | 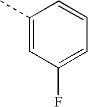 | 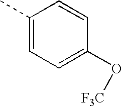 |
| LC-MS | 0.96 | 0.97 | 1.00 |
| t_{R} (min) | 620.54 | 608.50 | 674.42 |
| [M + H]⁺ | | | |
| Ex. No: | 56 | 57 | 58 |
| R^{A} | 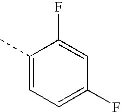 | 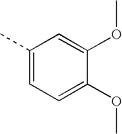 | 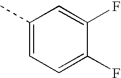 |
| LC-MS | 0.97 | 0.95 | 0.98 |
| t_{R} (min) | 626.42 | 650.43 | 626.40 |
| [M + H]⁺ | | | |

TABLE 3

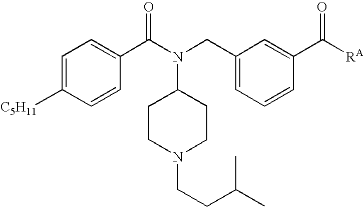

| Ex. No: | 59 | 60 | 61 |
|---|---|---|---|
| R^{A} | 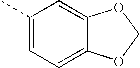 | 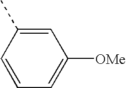 | 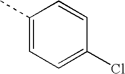 |
| LC-MS | 0.98 | 0.98 | 1.01 |
| t_{R} (min) | 583.10 | 569.13 | 573.08 |
| [M + H]⁺ | | | |
| Ex. No: | 62 | 63 | 64 |
| R^{A} | 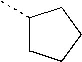 | 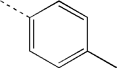 | 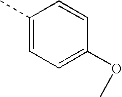 |
| LC-MS | 1.00 | 1.01 | 6.99 |
| t_{R} (min) | 531.18 | 553.12 | 569.13 |
| [M + H]⁺ | | | |
| Ex. No: | 65 | 66 | 67 |
| R^{A} | 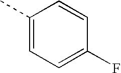 | 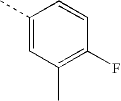 | 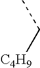 |
| LC-MS | 0.99 | 1.01 | 1.02 |
| t_{R} (min) | 557.10 | 571.13 | 533.18 |
| [M + H]⁺ | | | |
| Ex. No: | 68 | 69 | |
| R^{A} | 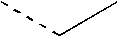 | 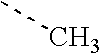 | |
| LC-MS | 0.95 | 0.93 | |
| t_{R} (min) | 491.17 | 477.15 | |
| [M + H]⁺ | | | |

TABLE 4

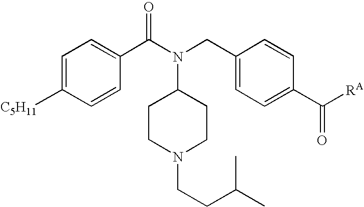

| Ex. No: | 70 | 71 | 72 |
|---|---|---|---|
| R^{A} | 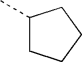 | 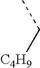 | 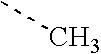 |
| LC-MS | 1.01 | 1.03 | 1.00 |
| t_{R} (min) | 531.16 | 533.18 | 477.65 |
| [M + H]⁺ | | | |
| Ex. No: | 73 | 74 | 75 |
| R^{A} | 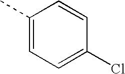 | 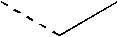 | 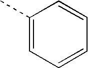 |
| LC-MS | 1.02 | 0.99 | 1.01 |
| t_{R} (min) | 573.08 | 491.66 | 539.11 |
| [M + H]⁺ | | | |
| Ex. No: | 76 | 77 | 78 |
| R^{A} | 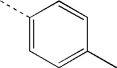 | 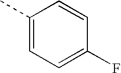 | 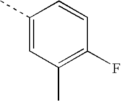 |
| LC-MS | 1.01 | 1.02 | 1.03 |
| t_{R} (min) | 553.12 | 557.8 | 571.74 |
| [M + H]⁺ | | | |

TABLE 5

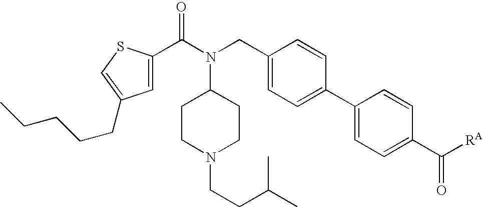

| Ex. No: | 79 | 80 | 81 |
|---|---|---|---|
| R^{A} | 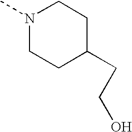 | 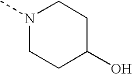 | 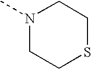 |
| LC-MS | 0.93 | 0.89 | 0.99 |
| t_{R} (min) | 672.68 | 644.57 | 646.50 |
| [M + H]⁺ | | | |
| Ex. No: | 82 | 83 | 84 |
| R^{A} | 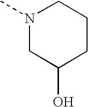 | 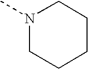 | 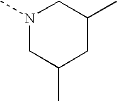 |
| LC-MS | 0.91 | 0.98 | 1.03 |
| t_{R} (min) | 644.63 | 628.65 | 656.66 |
| [M + H]⁺ | | | |
| Ex. No: | 85 | | |
| R^{A} | 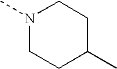 | | |
| LC-MS | 1.01 | | |
| t_{R} (min) | 642.58 | | |
| [M + H]⁺ | | | |

TABLE 6

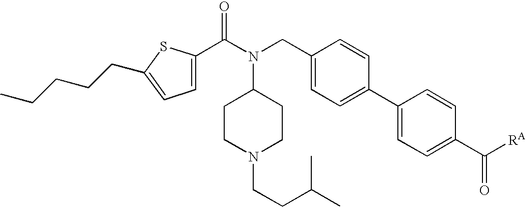

| Ex. No: | 86 | 87 | 88 |
|---|---|---|---|
| R^{A} | 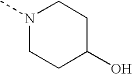 | 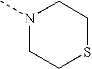 | 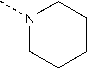 |
| LC-MS | 0.91 | 0.96 | 0.99 |
| t_{R} (min) | 644.66 | 646.46 | 628.69 |
| [M + H]⁺ | | | |
| Ex. No: | 89 | 90 | 91 |
| R^{A} | 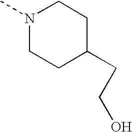 | 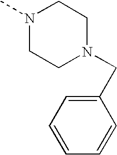 | 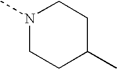 |
| LC-MS | 0.92 | 0.85 | 0.99 |
| t_{R} (min) | 672.71 | 720.06 | 642.68 |
| [M + H]⁺ | | | |
| Ex. No: | 92 | 93 | 94 |
| R^{A} | 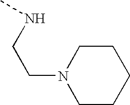 | 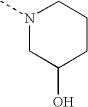 | 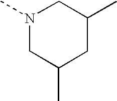 |
| LC-MS | 0.83 | 0.91 | 1.03 |
| t_{R} (min) | 671.75 | 644.64 | 656.70 |
| [M + H]⁺ | | | |
| Ex. No: | 95 | | |
| R^{A} | 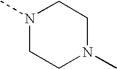 | | |
| LC-MS | 0.80 | | |
| t_{R} (min) | 643.65 | | |
| [M + H]⁺ | | | |

TABLE 7

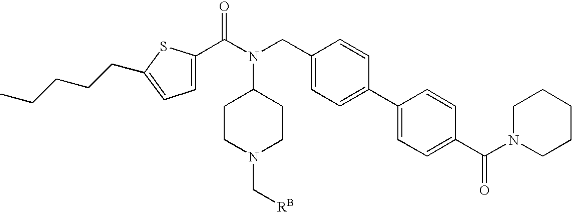

| Ex. No: | 96 | 97 | 98 |
|---|---|---|---|
| R^{A} | 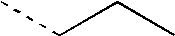 | 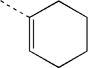 | 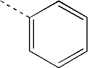 |
| LC-MS | 0.97 | 1.00 | 0.98 |
| t_{R} (min) | 614.56 | 652.63 | 648.56 |
| [M + H]⁺ | | | |
| Ex. No: | 99 | 100 | 101 |
| R^{A} | 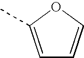 | 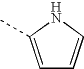 | 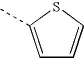 |
| LC-MS | 0.96 | 0.96 | 0.97 |
| t_{R} (min) | 638.57 | 637.60 | 654.54 |
| [M + H]⁺ | | | |
| Ex. No: | 102 | 103 | 104 |
| R^{A} | 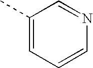 | 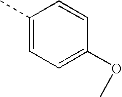 | 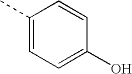 |
| LC-MS | 0.90 | 0.98 | 0.95 |
| t_{R} (min) | 649.58 | 678.67 | 664.61 |
| [M + H]⁺ | | | |
| Ex. No: | 105 | 106 | 107 |
| R^{A} | 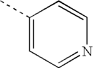 | 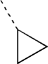 | 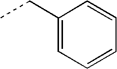 |
| LC-MS | 0.89 | 0.96 | 0.99 |
| t_{R} (min) | 649.52 | 612.53 | 662.57 |
| [M + H]⁺ | | | |
| Ex. No: | 108 | 109 | |
| R^{A} | 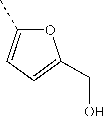 | 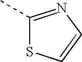 | |
| LC-MS | 0.93 | 0.94 | |
| t_{R} (min) | 668.68 | 655.52 | |
| [M + H]⁺ | | | |

TABLE 8

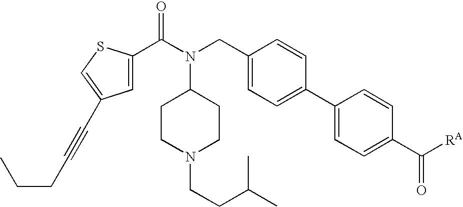

| Ex. No: | 110 | 111 | 112 |
|---|---|---|---|
| R^{A} | 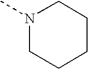 | 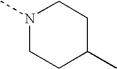 | 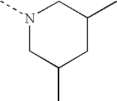 |
| LC-MS | 0.96 | 0.99 | 1.00 |
| t_{R} (min) | 624.52 | 638.57 | 652.65 |
| [M + H]⁺ | | | |
| Ex. No: | 113 | 114 | 115 |
| R^{A} | 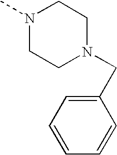 | 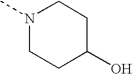 | 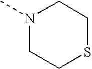 |
| LC-MS | 0.83 | 0.88 | 0.95 |
| t_{R} (min) | 715.66 | 640.60 | 642.47 |
| [M + H]⁺ | | | |
| Ex. No: | 116 | 117 | 118 |
| R^{A} | 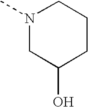 | 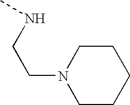 | 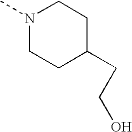 |
| LC-MS | 0.89 | 0.81 | 0.90 |
| t_{R} (min) | 640.62 | 667.59 | 668.67 |
| [M + H]⁺ | | | |
| Ex. No: | 119 | | |
| R^{A} | 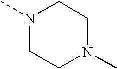 | | |
| LC-MS | 0.78 | | |
| t_{R} (min) | 639.54 | | |
| [M + H]⁺ | | | |

TABLE 9

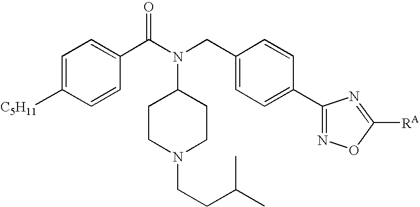

| Ex. No: | 120 | 121 | 122 |
|---|---|---|---|
| R^{A} | 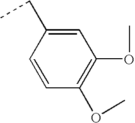 | 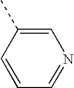 | 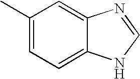 |
| LC-MS | 0.98 | 1.00 | 0.88 |
| t_{R} (min) | 653.48 | 580.48 | 521.40 |
| [M + H]⁺ | | | |
| Ex. No: | 123 | 124 | 125 |
| R^{A} | 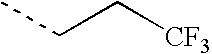 | 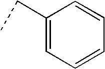 | 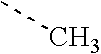 |
| LC-MS | 1.00 | 1.02 | 0.95 |
| t_{R} (min) | 599.48 | 593.46 | 517.42 |
| [M + H]⁺ | | | |
| Ex. No: | 126 | 127 | 128 |
| R^{A} | 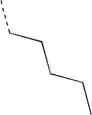 | 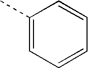 | 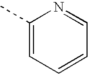 |
| LC-MS | 1.04 | 1.02 | 0.98 |
| t_{R} (min) | 573.47 | 579.37 | 580.47 |
| [M + H]⁺ | | | |
| Ex. No: | 129 | 130 | 131 |
| R^{A} | 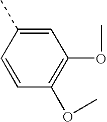 | 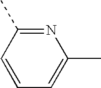 | 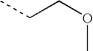 |
| LC-MS | 1.03 | 0.99 | 0.96 |
| t_{R} (min) | 639.43 | 595.41 | 561.45 |
| [M + H]⁺ | | | |
| Ex. No: | 132 | 133 | 134 |
| R^{A} | 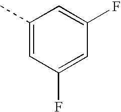 | 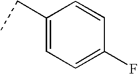 | 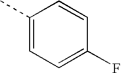 |
| LC-MS | 1.04 | 1.01 | 1.03 |
| t_{R} (min) | 615.40 | 611.43 | 597.39 |
| [M + H]⁺ | | | |
| Ex. No: | 135 | 136 | |
| R^{A} | 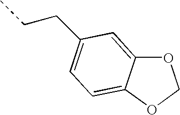 | 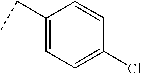 | |
| LC-MS | 1.20 | 1.03 | |
| t_{R} (min) | 729.50 | 627.43 | |
| [M + H]⁺ | | | |

TABLE 10

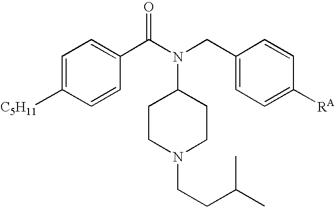

| Ex. No: | 137 | 138 | 139 |
|---|---|---|---|
| R^{A} | 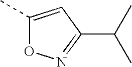 | 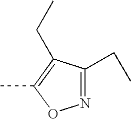 | 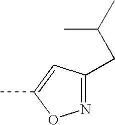 |
| LC-MS | 0.99 | 1.01 | 1.00 |
| t_{R} (min) | 544.45 | 558.43 | 558.02 |
| [M + H]⁺ | | | |
| Ex. No: | 140 | 141 | 142 |
| R^{A} | 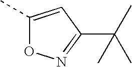 | 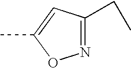 | 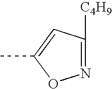 |
| LC-MS | 1.00 | 0.98 | 1.02 |
| t_{R} (min) | 558.46 | 530.55 | 558.44 |
| [M + H]⁺ | | | |
| Ex. No: | 143 | 144 | 145 |
| R^{A} | 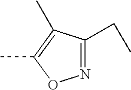 | 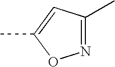 | 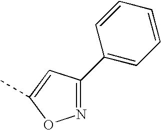 |
| LC-MS | 0.99 | 0.97 | 1.02 |
| t_{R} (min) | 544.44 | 516.43 | 578.43 |
| [M + H]⁺ | | | |

TABLE 11

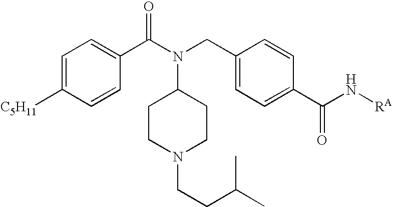

| Ex. No: | 146 | 147 | 148 |
|---|---|---|---|
| R^{A} | 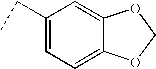 | 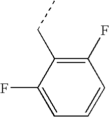 | 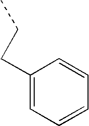 |
| LC-MS | 0.94 | 0.97 | 0.96 |
| t_{R} (min) | 612.51 | 604.45 | 582.45 |
| [M + H]⁺ | | | |
| Ex. No: | 149 | 150 | 151 |
| R^{A} | 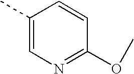 | 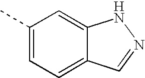 | 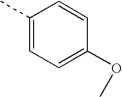 |
| LC-MS | 0.94 | 0.96 | 0.96 |
| t_{R} (min) | 585.49 | 622.44 | 584.44 |
| [M + H]⁺ | | | |
| Ex. No: | 152 | 153 | 154 |
| R^{A} | 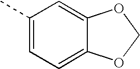 | 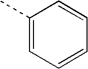 | 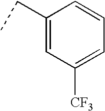 |
| LC-MS | 0.96 | 0.97 | 1.00 |
| t_{R} (min) | 598.46 | 554.41 | 636.46 |
| [M + H]⁺ | | | |
| Ex. No: | 155 | 156 | 157 |
| R^{A} | 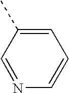 | 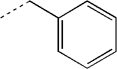 | 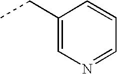 |
| LC-MS | 0.82 | 0.96 | 0.78 |
| t_{R} (min) | 555.41 | 568.48 | 569.56 |
| [M + H]⁺ | | | |
| Ex. No: | 158 | 159 | 160 |
| R^{A} | 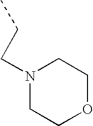 | 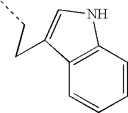 | 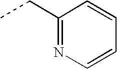 |
| LC-MS | 0.79 | 0.96 | 0.81 |
| t_{R} (min) | 705.51 | 621.49 | 569.47 |
| [M + H]⁺ | | | |

TABLE 12

| | | |
|---|---|---|
| Example 161 | 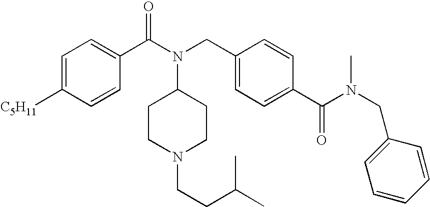 | LC-MS: t_{R} (min): 0.97 [M + H]⁺: 582.45 |

TABLE 13

| | | |
|---|---|---|
| Example 162 | 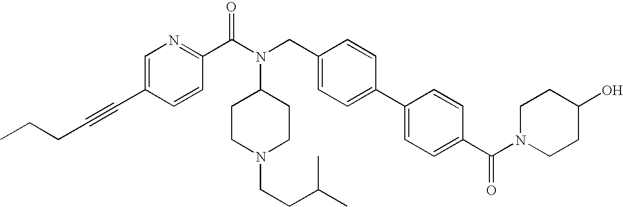 | LC-MS: t_{R} (min): 0.86 [M + H]⁺: 635.67 |
| Example 163 | 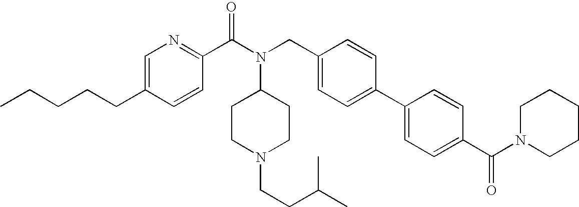 | LC-MS: t_{R} (min): 0.92 [M + H]⁺: 623.89 |
| Example 164 | 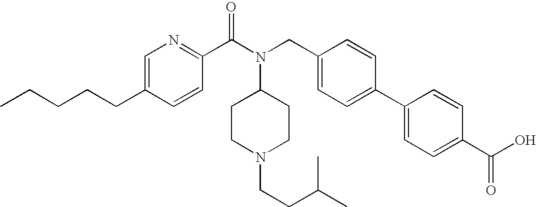 | LC-MS: t_{R} (min): 0.90 [M + H]⁺: 556.56 |

TABLE 14

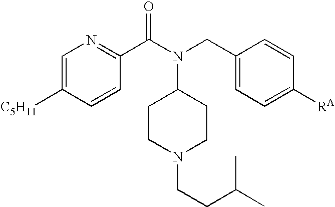

| Ex. No: | 165 | 166 | 167 |
|---|---|---|---|
| R^{A} | 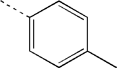 | 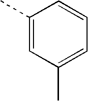 | 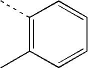 |
| LC-MS | 0.96 | 0.98 | 0.97 |
| t_{R} (min) | 526.23 | 526.24 | 526.25 |
| [M + H]⁺ | | | |
| Ex. No: | 168 | 169 | 170 |
| R^{A} | 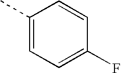 | 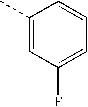 | 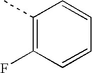 |
| LC-MS | 0.96 | 0.97 | 0.96 |
| t_{R} (min) | 530.21 | 530.22 | 530.23 |
| [M + H]⁺ | | | |
| Ex. No: | 171 | 172 | 173 |
| R^{A} | 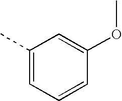 | 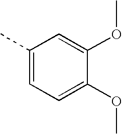 | 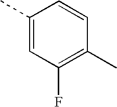 |
| LC-MS | 0.98 | 0.96 | 1.00 |
| t_{R} (min) | 542.47 | 572.63 | 544.48 |
| [M + H]⁺ | | | |
| Ex. No: | 174 | 175 | 176 |
| R^{A} | 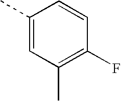 | 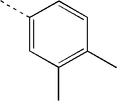 | 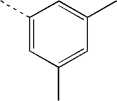 |
| LC-MS | 1.00 | 1.01 | 1.01 |
| t_{R} (min) | 544.42 | 540.48 | 540.58 |
| [M + H]⁺ | | | |
| Ex. No: | 177 | 178 | 179 |
| R^{A} | 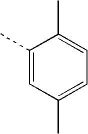 | 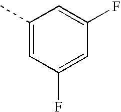 | 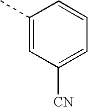 |
| LC-MS | 1.01 | 0.99 | 0.96 |
| t_{R} (min) | 540.57 | 547.54 | 537.54 |
| [M + H]⁺ | | | |
| Ex. No: | 180 | 181 | 182 |
| R^{A} | 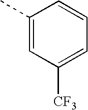 | 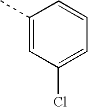 | 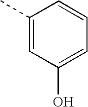 |
| LC-MS | 1.01 | 1.00 | 0.92 |
| t_{R} (min) | 580.62 | 546.54 | 528.58 |
| [M + H]⁺ | | | |

TABLE 15

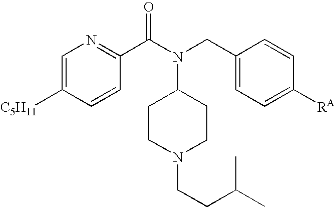

| Ex. No: | 183 | 184 | 185 |
|---|---|---|---|
| R^{A} | 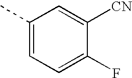 | 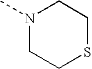 | 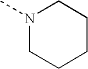 |
| LC-MS | 0.98 | 0.87 | 0.75 |
| t_{R} (min) | 555.60 | 537.21 | 519.27 |
| [M + H]⁺ | | | |
| Ex. No: | 186 | 187 | 188 |
| R^{A} | 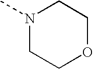 | 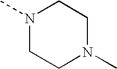 | 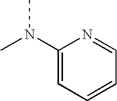 |
| LC-MS | 0.86 | 0.73 | 0.76 |
| t_{R} (min) | 521.2 | 534.26 | 524.21 |
| [M + H]⁺ | | | |
| Ex. No: | 189 | 190 | 191 |
| R^{A} | 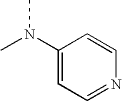 | 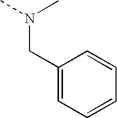 | 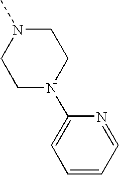 |
| LC-MS | 0.75 | 0.93 | 0.77 |
| t_{R} (min) | 542.2 | 555.2 | 597.26 |
| [M + H]⁺ | | | |
| Ex. No: | 192 | 193 | |
| R^{A} | 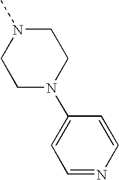 | 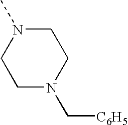 | |
| LC-MS | 0.76 | 0.80 | |
| t_{R} (min) | 597.28 | 610.28 | |
| [M + H]⁺ | | | |
| Ex. No: | 194 | 195 | 196 |
| R^{A} | 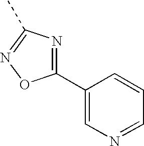 | 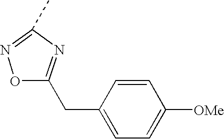 | 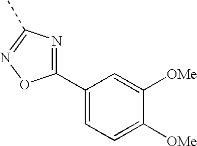 |
| LC-MS | 0.97 | 1.00 | 0.99 |
| t_{R} (min) | 581.59 | 624.60 | 640.60 |
| [M + H]⁺ | | | |
| Ex. No: | 197 | 198 | |
| R^{A} | 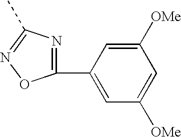 | 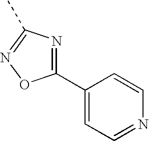 | |
| LC-MS | 1.03 | 0.97 | |
| t_{R} (min) | 640.54 | 581.55 | |
| [M + H]⁺ | | | |

TABLE 16

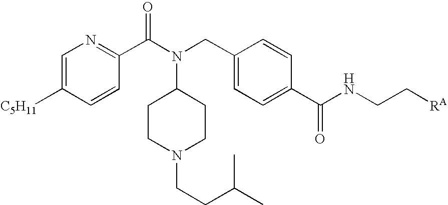

| Ex. No: | 199 | 200 | 201 |
|---|---|---|---|
| R^{A} | 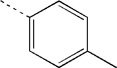 | 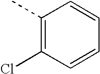 | 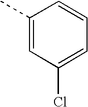 |
| LC-MS | 0.94 | 0.97 | 0.97 |
| t_{R} (min) | 583.51 | 617.56 | 617.60 |
| [M + H]⁺ | | | |
| Ex. No: | 202 | 203 | 204 |
| R^{A} | 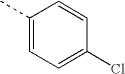 | 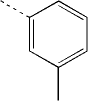 | 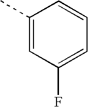 |
| LC-MS | 0.97 | 0.96 | 0.95 |
| t_{R} (min) | 617.60 | 597.67 | 601.57 |
| [M + H]⁺ | | | |
| Ex. No: | 205 | 206 | 207 |
| R^{A} | 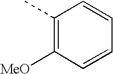 | 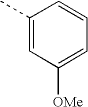 | 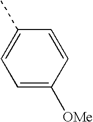 |
| LC-MS | 0.95 | 0.94 | 0.94 |
| t_{R} (min) | 613.68 | 613.59 | 613.66 |
| [M + H]⁺ | | | |
| Ex. No: | 208 | 209 | |
| R^{A} | 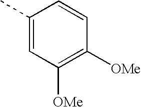 | 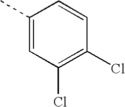 | |
| LC-MS | 0.92 | 0.99 | |
| t_{R} (min) | 643.61 | 651.56 | |
| [M + H]⁺ | | | |

TABLE 17

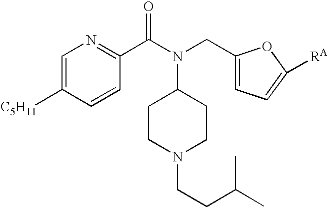

| Ex. No: | 210 | 211 | 212 |
|---|---|---|---|
| R^{A} | 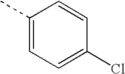 | 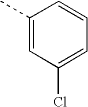 | 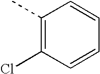 |
| LC-MS | 0.97 | 0.98 | 0.97 |
| t_{R} (min) | 536.22 | 536.21 | 536.22 |
| [M + H]⁺ | | | |
| Ex. No: | 213 | 214 | 215 |
| R^{A} | 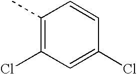 | 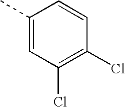 | 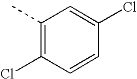 |
| LC-MS | 1.00 | 1.00 | 1.00 |
| t_{R} (min) | 570.18 | 570.18 | 570.18 |
| [M + H]⁺ | | | |

TABLE 18

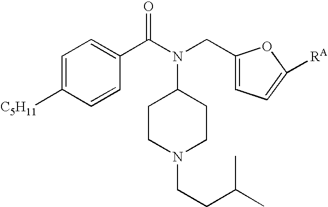

| Ex. No: | 216 | 217 | 218 |
|---|---|---|---|
| R^{A} | 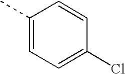 | 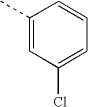 | 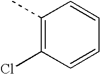 |
| LC-MS | 1.01 | 1.01 | 1.01 |
| t_{R} (min) | 535.25 | 535.26 | 535.24 |
| [M + H]⁺ | | | |
| Ex. No: | 219 | 220 | 221 |
| R^{A} | 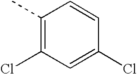 | 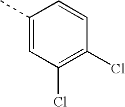 | 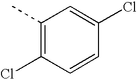 |
| LC-MS | 1.03 | 1.03 | 1.03 |
| t_{R} (min) | 569.20 | 569.19 | 569.21 |
| [M + H]⁺ | | | |

TABLE 19

| | | |
|---|---|---|
| Example 222 | 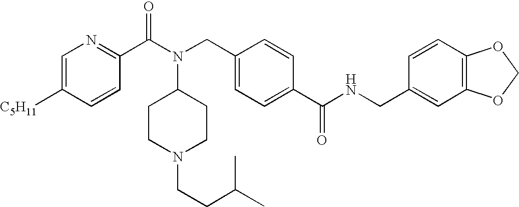 | LC-MS: t_{R} (min): 0.93 [M + H]⁺: 613.57 |
| Example 223 | 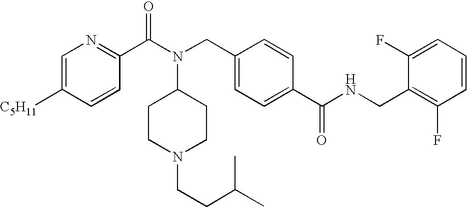 | LC-MS: t_{R} (min): 0.94 [M + H]⁺: 605.54 |
| Example 224 | 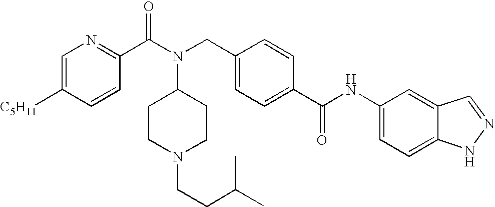 | LC-MS: t_{R} (min): 0.90 [M + H]⁺: 595.68 |
| Example 225 | 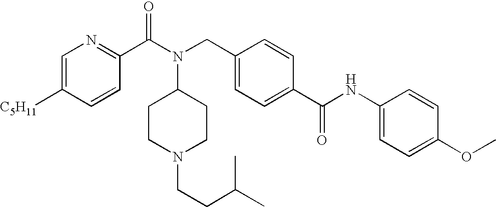 | LC-MS: t_{R} (min): 0.93 [M + H]⁺: 585.61 |
| Example 226 | 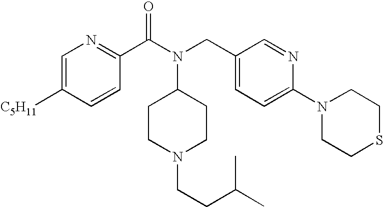 | LC-MS: t_{R} (min): 0.78 [M + H]⁺: 538.18 |
| Example 227 | 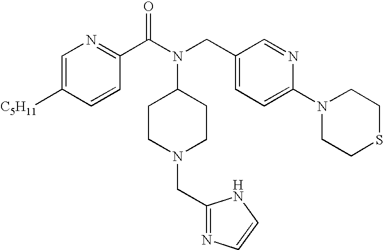 | LC-MS: t_{R} (min): 0.69 [M + H]⁺: 548.13 |
| Example 228 | 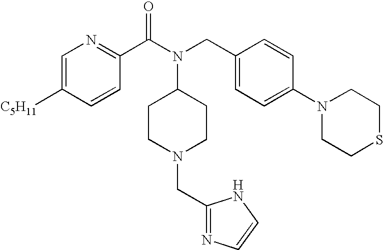 | LC-MS: t_{R} (min): 0.74 [M + H]⁺: 547.15 |

TABLE 20

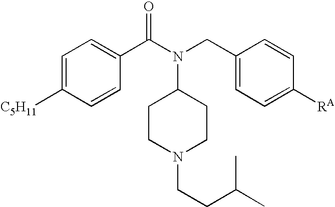

| Ex. No: | 229 | 230 | 231 |
|---|---|---|---|
| R^{A} | 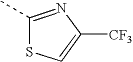 | 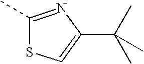 | 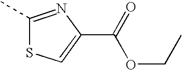 |
| LC-MS | 1.01 | 1.05 | 0.98 |
| t_{R} (min) | 586.41 | 574.09 | 591.31 |
| [M + H]⁺ | | | |
| Ex. No: | 232 | | |
| R^{A} | 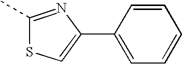 | | |
| LC-MS | 1.04 | | |
| t_{R} (min) | 594.46 | | |
| [M + H]⁺ | | | |

The Fluorescence Resonance Energy Transfer (FRET) Assay for Plasmepsin II & IV and Human Cathepsin D & E:

The assay conditions are selected according to reports in the literature.

The FRET assay is performed in white polysorp plates (Fluoronunc, cat no 264 572) at 37° C. with a final volume of 80 μl.

The assay buffer is composed of 50 mM sodium acetate pH 5, 12.5% (w/v) glycerol, and 0.1% (w/v) BSA. The reaction consists of the following components: 60 μl assay buffer, 4 μl inhibitor (in DMSO), 8 μl substrate (M-2120 from BACHEM) to a final concentration of 1 μM and 8 μl enzyme (plasmepsin II, plasmepsin IV or cathepsin E to a final amount of 0.015 μg/ml per assay tube, cathepsin D to a final amount of 0.05 μg/ml per assay tube). The inhibitor is pre-diluted in DMSO in a dilution plate and six concentrations are prepared in duplicate. The compounds are usually tested at a final concentration varying from 1 nM to 100 μM. The substrate is diluted using 50% DMSO-50% assay buffer and the enzyme using assay buffer. The mixtures are then incubated for 3 h at 37° C. and the fluorescence is determined at 1 and 3 hour with a FluoroStar Galaxy from BMG using excitation and emission filters of 355 and 520 nm, respectively.

Auto-fluorescence of all the test substances is determined in assay buffer in the absence of substrate and enzyme and this value is subtracted from the final signal.

The inhibitory activity of the compounds is expressed as IC₅₀, which represents the concentration of compound that inhibits 50% of the maximal (uninhibited) enzyme activity.

TABLE
IC₅₀-values of selected examples:

| Example Number | IC₅₀ (PM II in nM) | IC₅₀ (PM IV in nM) |
|---|---|---|
| 1 | 22 | 434 |
| 19 | 50 | 580 |
| 25 | 60 | 495 |
| 79 | 43 | not determined |
| 88 | 77 | not determined |
| 137 | 16 | 897 |
| 140 | 43 | not determined |
| 146 | 14 | 1817 |
| 151 | 44 | not determined |
| 163 | 10 | 635 |
| 165 | 22 | 167 |
| 166 | 16 | 22 |
| 169 | 18 | 27 |
| 180 | 48 | 406 |
| 190 | 40 | 1877 |
| 196 | 80 | 3441 |
| 218 | 110 | 276 |
| 220 | 41 | 56 |
| 223 | 40 | 1106 |
| 226 | 51 | 359 |

In Vitro Antimalarial Activity: Plasmodium falciparum In Vitro Assay

In vitro activity against erythrocytic stages of P. falciparum is determined using a [³H] incorporation assay. One strain resistant to chloroquine and e (P. falciparum K1) is used in the assays, and all test compounds are compared for activity with the standard drugs chloroquine (sigma C6628) and artemisinin (sigma-36, 159-3). Compounds are diluted in DMSO to 1 mM and added to parasite cultures incubated in RPMI 1640 medium without hypoxanthine, supplemented with HEPES (5.94 g/L), NaHCO₃ (2.1 g/L), neomycin (100 U/mL), Albumax (5 g/L) and washed human red cells at 2.5% haematocrit (0.3% parasitaemia). Seven serial doubling dilutions of each drug are prepared in 96-well microtitre plates and incubated in a humidifying atmosphere at 37° C.; 4% CO₂, 3% O₂, 93% N₂.

After 48 hours, 50 μl of [³H] hypoxanthine (0.5 μCi) is added to each well of a plate. The plates are incubated for a further 24 hours under the same conditions. The plates are then harvested with a Betaplate cell harvester (Wallac) and washed with distilled water. The dried filters are inserted into a plastic foil with 10 mL of scintillation fluid, and counted in a Betaplate liquid scintillation counter. IC₅₀ values are calculated from sigmoidal inhibition curves using Microsoft Excel.

In Vivo Antimalarial Efficacy Studies

In vivo antimalarial activity is assessed for groups of three female NMRI mice (20-22 g) intravenously infected on day 0 with P. berghei strain GFP-ANKA (0.2 mL heparinized saline suspension containing 2×10⁷ parasitized erythrocytes). In control mice, parasitaemia typically rise to approximately 40% by day 3 after infection, and control mice die between day 5 and day 7 after infection. For the mice treated with compounds, compounds are either formulated in an aqueous-gelatine vehicle with 3 mg/mL compounds or in tween 80/ethanol (7%/3%) with 5 mg/mL.

Compounds are administered intraperitonealy or subcoutaneously either as two consecutive twice-daily dosings (BID) (2×75 mg/kg BID, 24 and 48 hours after infection) or as four consecutive daily doses (4×10 mg/kg or 4×50 mg/kg, 3, 24, 48 and 72 hours after infection). With the double BID-dose regimen, 24 hours after the last drug treatment, 1 μl tail blood is taken, resuspended in 1 mL PBS buffer and parasitemia determined with a FACScan (Becton Dickinson) by counting 100 000 red blood cells. Tail blood samples for the quadruple-dose regimen are processed on day 4 after infection. Activity is calculated as the difference between the mean value of the control and treated groups expressed as a percent relative to the control group. For parasetimias lower than 0.1%, the presence of parasites in the FACS gate is checked visually. The survival days of infected mice treated with compound is also recorded for each compound. Mice surviving for 30 days are checked for parasitemia and subsequently euthanized. A compound is considered curative if the animal survives to day 30 post-infection with no detectable parasites.

1. A compound selected from the group consisting of 4-aminopiperidine compounds of the formula I: 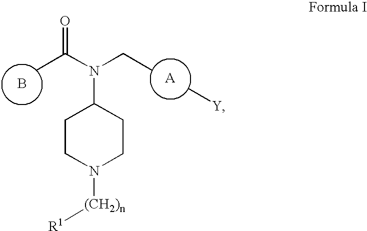
wherein
R¹ represents hydrogen; alkyl; alkenyl; alkynyl; cyclopropyl; cyclopentyl; cyclohexyl;
cyclohexenyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, and bromine; thienyl that can be mono-substituted with methyl or chlorine; benzothienyl; benzofuranyl; quinolinyl; isoquinolinyl; benzo[1,3]dioxol-5-yl; methoxy-benzo[1,3]dioxol-5-yl; chloro-benzo[1,3]dioxol-5-yl; 2,2-diphenyl-ethyl; 2-phenyl-propyl; 1-[2,6,6-trimethyl-cyclohex-1-enyl]-methyl; pyrrolyl; thiazolyl; or imidazolyl,
n represents the integer 1, 2, or 3, 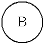
represents 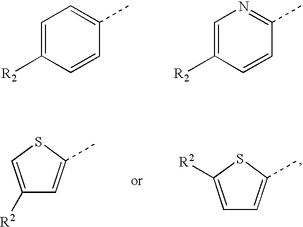
R² represents butyl, pentyl or hexyl; or 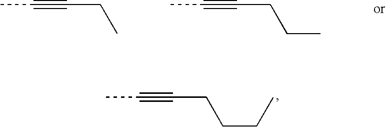
In case 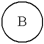
represents 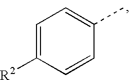
Y represents 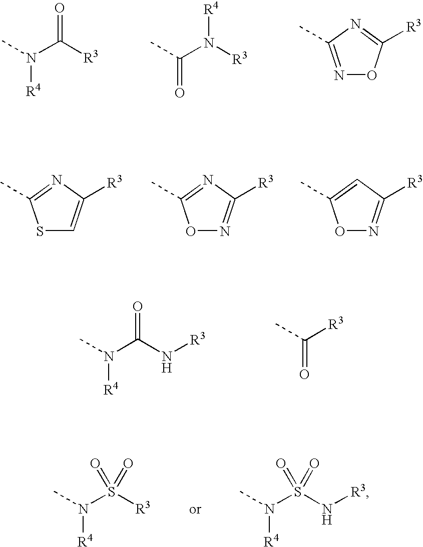
In case 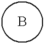
represents 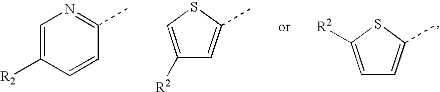
or in case 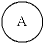
represents 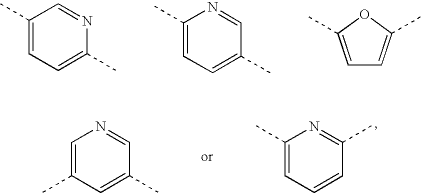
Y represents 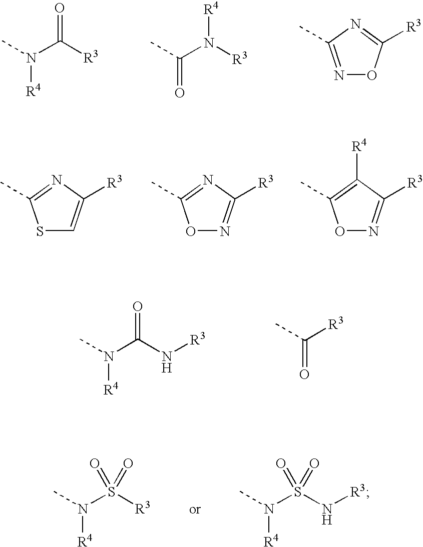
or phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, carboxyl, or the following radicals: 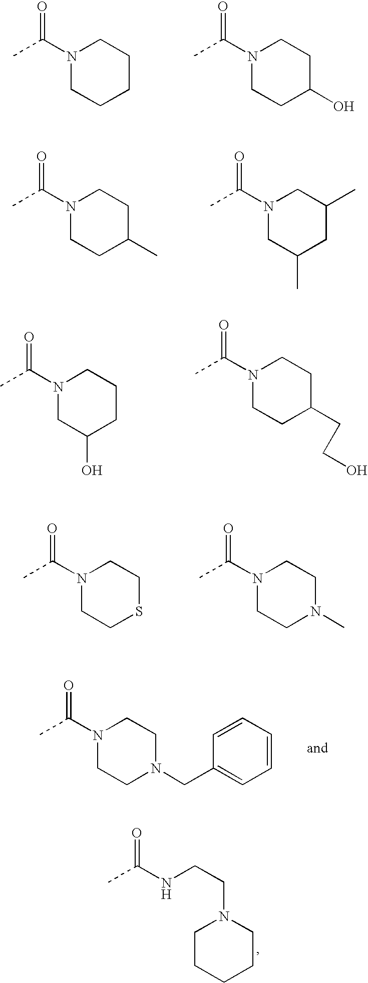
or in case 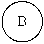
represents 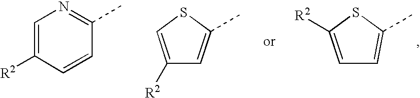
or in case 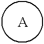
represents 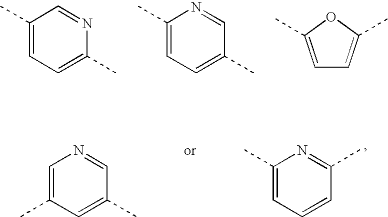
Y can also represent pyridyl that can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, and bromine; thienyl that can be mono-substituted with methyl or chlorine; benzothienyl; benzofuranyl; quinolinyl; isoquinolinyl; benzo[1,3]dioxolyl; 2,2-diphenyl-ethyl; 2-phenyl-propyl; 1-[2,6,6-trimethyl-cyclohex-1-enyl]-methyl; 3-methyl-butyl; phenoxy, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl-oxy, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; or the following radical: 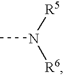
R³ represents alkyl; cycloalkyl; —CF₃; CF₃-alkyl-; alkoxy-alkyl; alkoxy-carbonyl; carboxyl; benzo[1,3]dioxol-5-yl; methoxy-benzo[1,3]dioxol-5-yl; chloro-benzo[1,3]dioxol-5-yl; benzo[1,3]dioxol-5-yl-alkyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenyl-alkyl, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenoxy-methyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; pyridyl-alkyl, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, —CF₃ and halogen; thienyl that can be mono- or di-substituted, wherein the substituents are independently selected from methyl and halogen; thienyl-alkyl; pyrazolyl that can be mono- or di-substituted, wherein the substituents are independently selected from methyl and halogen; benzothienyl; benzofuranyl; benzimidazolyl; benzopyrazolyl; indolyl; indolyl-alkyl; or morpholinyl-alkyl; or in case
Y represents 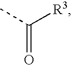
R³ in addition to the above mentioned possibilities may also represent thiomorpholinyl; piperidinyl that can be mono- or di-substituted, wherein the substituents are independently selected from alkyl, hydroxy-alkyl, and hydroxy; piperidinyl-alkyl; morpholinyl; or 1-piperazinyl which can be substituted at the nitrogen atom at position 4 with alkyl, benzyl, pyridyl, or phenyl, wherein the phenyl group can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl,
R⁴ represents hydrogen; methyl; ethyl; isopropyl; or cyclopropyl,
R⁵ and R⁶ represent hydrogen; alkyl; cycloalkyl; phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; phenyl-alkyl, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyrrolidinyl-alkyl; pyridyl that can be mono-, di-, or tri-substituted, wherein the substituents are independently selected from halogen, alkyl, alkoxy, —CF₃, —OCF₃, hydroxy, alkoxy-carbonyl, and carboxyl; pyridyl-alkyl, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; piperidinylalkyl; morpholinyl-alkyl; 4-methyl-piperazinyl-alkyl; 4-benzyl-piperazinyl-alkyl; alkoxy-alkyl or bis-alkyl-amino-alkyl and may be the same or different; or R⁵ and R⁶ can together form a morpholinyl ring; a thiomorpholinyl ring; a piperidinyl ring which can be mono- or di-substituted, wherein the substituents are independently selected from methyl and hydroxy; or 1-piperazinyl which can be substituted at the nitrogen atom at position 4 with alkyl, benzyl, pyridyl, or phenyl, wherein the phenyl group can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl.
and 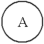
represents 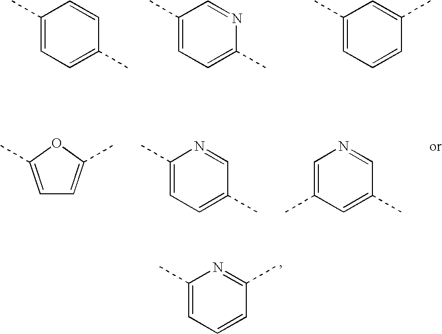
or an optically pure enantiomer, a mixture of enantiomers, a racemate, a diastereomer, a mixture of diastereomers, a diastereomeric racemate, a mixture of diastereomeric racemates, or a meso-form, a salt, a solvent complex, or a morphological form, thereof.

2. A compound according to claim 1, which is a compound of the formula II: 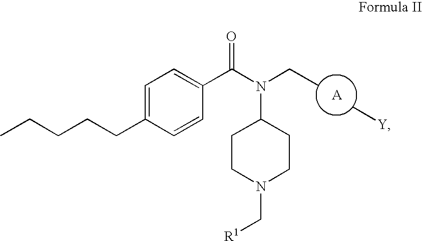
wherein
Y represents

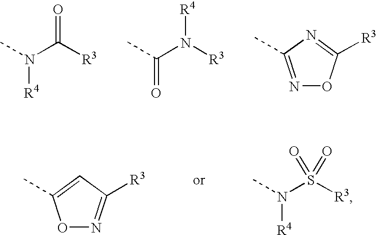

3. A compound according to claim 1, which is a compound of the formula III: 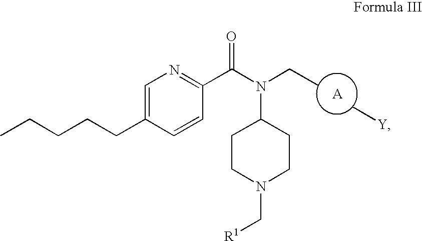
wherein
Y represents 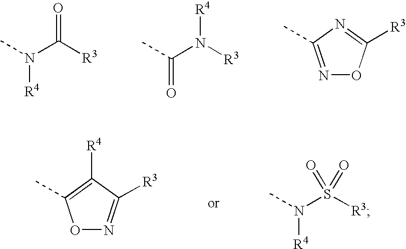
or phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, carboxyl, or the following radicals: 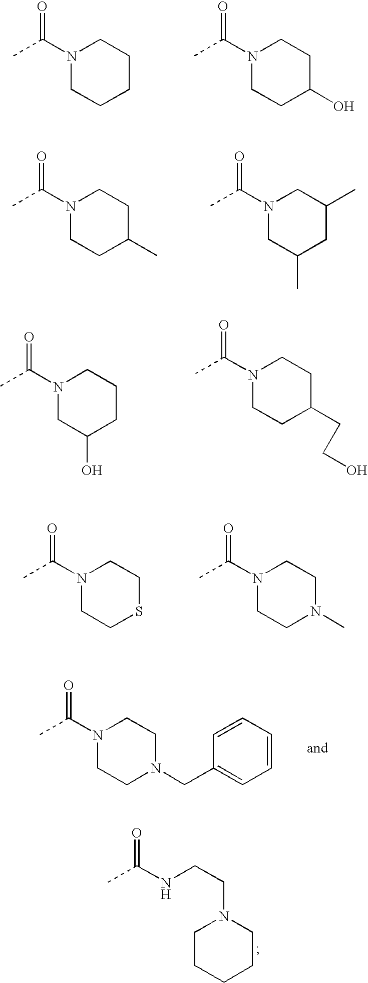
or Y represents pyridyl that can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, and bromine; thienyl that can be mono-substituted with methyl or chlorine; benzothienyl; benzofuranyl; quinolinyl; isoquinolinyl; benzo[1,3]dioxolyl; 2,2-diphenyl-ethyl; 2-phenyl-propyl; 1-[2,6,6-trimethyl-cyclohex-1-enyl]-methyl; 3-methyl-butyl; phenoxy, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl-oxy, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; or the following radical:

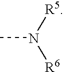

4. A compound according to claim 1, which is a compound of the formula IV: 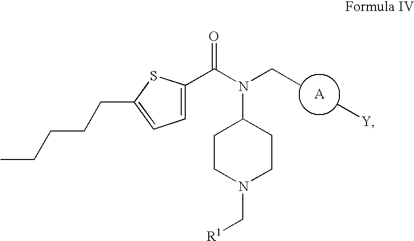
wherein
Y represents 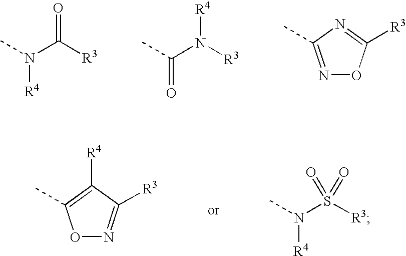
or phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, carboxyl, or the following radicals: 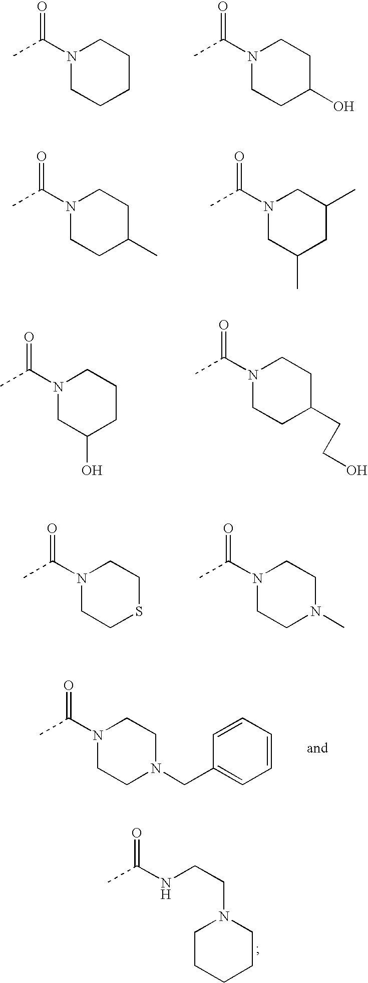
or Y represents pyridyl that can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, and bromine; thienyl that can be mono-substituted with methyl or chlorine; benzothienyl; benzofuranyl; quinolinyl; isoquinolinyl; benzo[1,3]dioxolyl; 2,2-diphenyl-ethyl; 2-phenyl-propyl; 1-[2,6,6-trimethyl-cyclohex-1-enyl]-methyl; 3-methyl-butyl; phenoxy, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl-oxy, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; or the following radical:

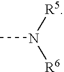

5. A compound according to claim 1, which is a compound of the formula V: 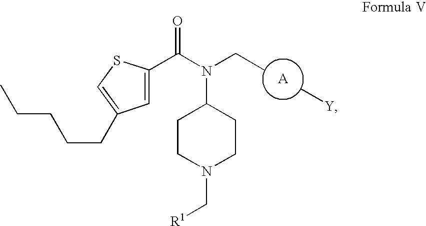
wherein
Y represents 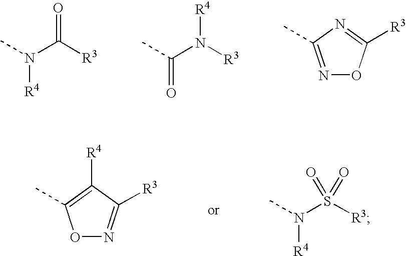
or phenyl that can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, carboxyl, or the following radicals: 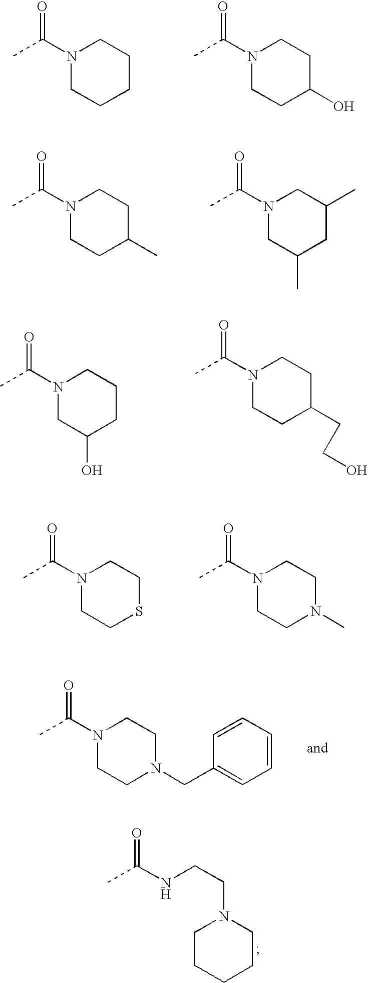
or Y represents pyridyl that can be mono-, di-, or tri-substituted, wherein the substituents are independently selected from halogen, alkyl, alkoxy, —CF₃, —OCF₃, hydroxy, alkoxy-carbonyl, and carboxyl; furanyl that can be mono-, or di-substituted, wherein the substituents are independently selected from methyl, hydroxy-methyl, and bromine; thienyl that can be mono-substituted with methyl or chlorine; benzothienyl; benzofuranyl; quinolinyl; isoquinolinyl; benzo[1,3]dioxolyl; 2,2-diphenyl-ethyl; 2-phenyl-propyl; 1-[2,6,6-trimethyl-cyclohex-1-enyl]-methyl; 3-methyl-butyl; phenoxy, wherein the phenyl ring can be mono-, di-, tri-, or tetra-substituted, wherein the substituents are independently selected from halogen, alkyl, alkoxy, —CF₃, —OCF₃, hydroxy, cyano, alkoxy-carbonyl, alkyl-carbonyl, and carboxyl; pyridyl-oxy, wherein the pyridyl ring can be mono-, di-, or tri-substituted, wherein the substituents are independently selected from halogen, alkyl, alkoxy, —CF₃, —OCF₃, hydroxy, alkoxy-carbonyl, and carboxyl; or the following radical:

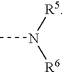

6. A compound according to claim 1, which is a compound of the formula VI: 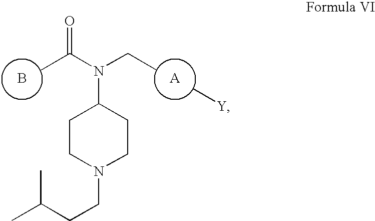
wherein R² represents pentyl or hexyl.

7. A compound according to claim 1, which is a compound of the formula VII: 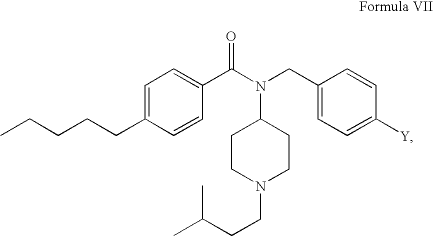
wherein Y represents

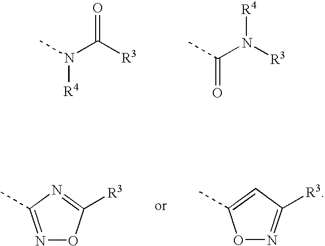

8. A compound according to claim 1, wherein
R¹ represents alkyl; cyclopropyl; cyclohexenyl; phenyl that can be mono-substituted with alkoxy or hydroxy; pyridyl; furanyl that can be mono-substituted with hydroxy-methyl; thienyl; pyrrolyl; thiazolyl; or imidazolyl,
n represents the integer 1, 2, or 3, 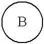
represents 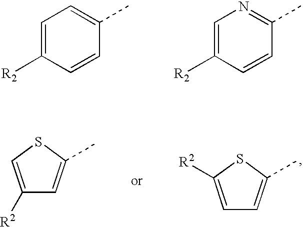
R² represents pentyl or 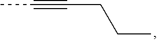
In case 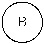
represents 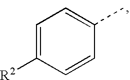
Y represents 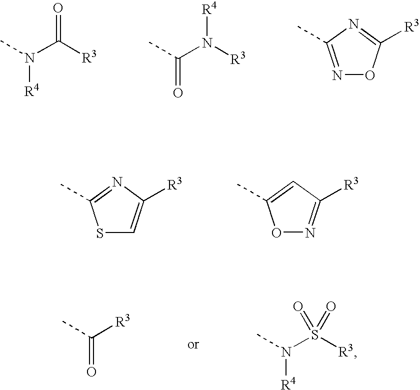
In case 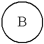
represents 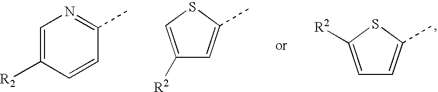
or in cased 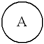
represents 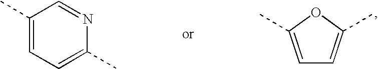
Y represents 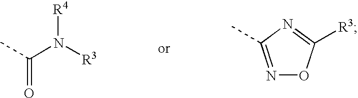
or phenyl that can be mono- or di-substituted, wherein the substituents are independently selected from halogen, alkyl, alkoxy, —CF₃, hydroxy, cyano, carboxyl, or the following radicals: 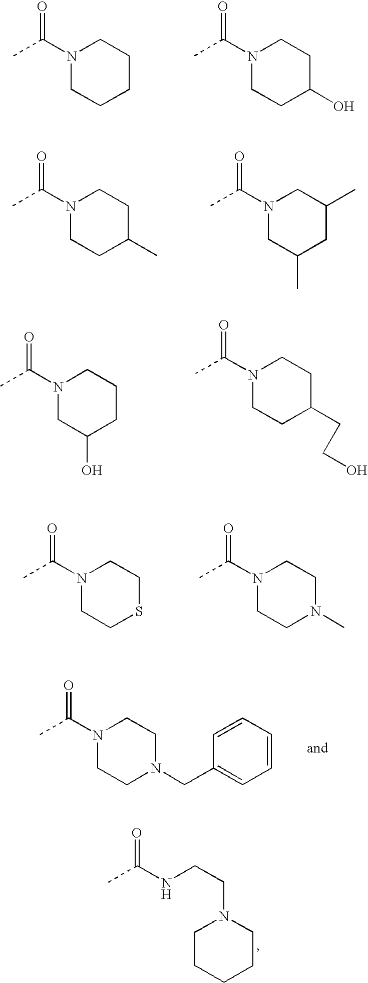
or in case 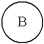
represents 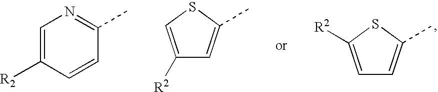
or in case 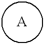
represents 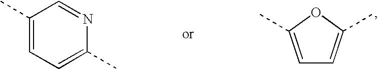
Y can also represent the following radical: 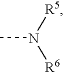
R³ represents alkyl; cycloalkyl; —CF₃; CF₃-alkyl-; alkoxy-alkyl; alkoxy-carbonyl; phenyl that can be mono-, di-, or tri-substituted, wherein the substituents are independently selected from halogen, alkyl, alkoxy, —CF₃, —OCF₃, and cyano; phenyl-alkyl, wherein the phenyl ring can be mono- or di-substituted, wherein the substituents are independently selected from alkyl, halogen, alkoxy, and —CF₃; phenoxy-methyl; pyridyl that can be mono-substituted with alkyl or alkoxy; pyridyl-alkyl; furanyl that can be di-substituted, wherein the substituents are independently selected from methyl and —CF₃; thienyl that can be mono- or di-substituted, wherein the substituents are independently selected from halogen; thienyl-alkyl; pyrazolyl that is di-substituted, wherein the substituents are independently selected from methyl and halogen; benzothienyl; benzimidazolyl; benzopyrazolyl; indolyl-alkyl; morpholinyl-alkyl; benzo[1,3]dioxol-5-yl; or benzo[1,3]dioxol-5-yl-alkyl,
R⁴ represents hydrogen or methyl,
R⁵ and R⁶ represent alkyl; phenyl-alkyl; or pyridyl; or R⁵ and R⁶ together form a morpholinyl ring; a thiomorpholinyl ring; a piperidinyl ring; or 1-piperazinyl which can be substituted at the nitrogen atom at position 4 with alkyl, benzyl, or pyridyl, and 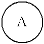
represents

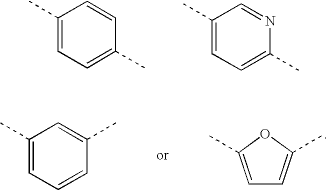

9. A compound according to claim 1 selected from the group consisting of:

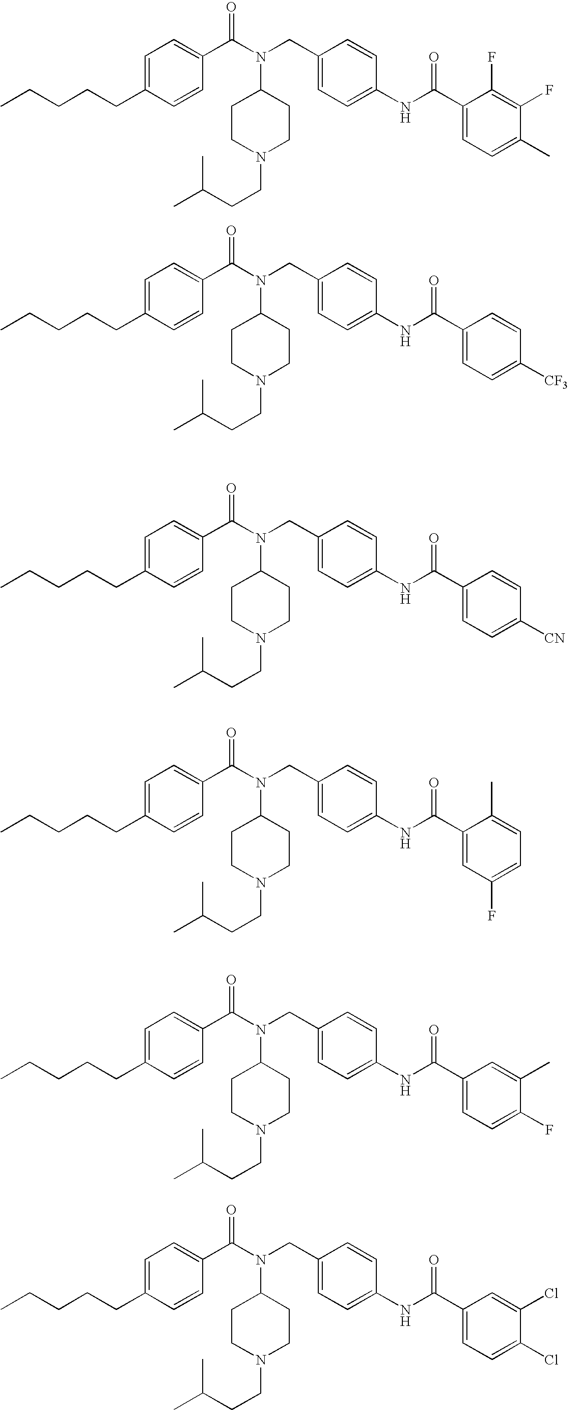

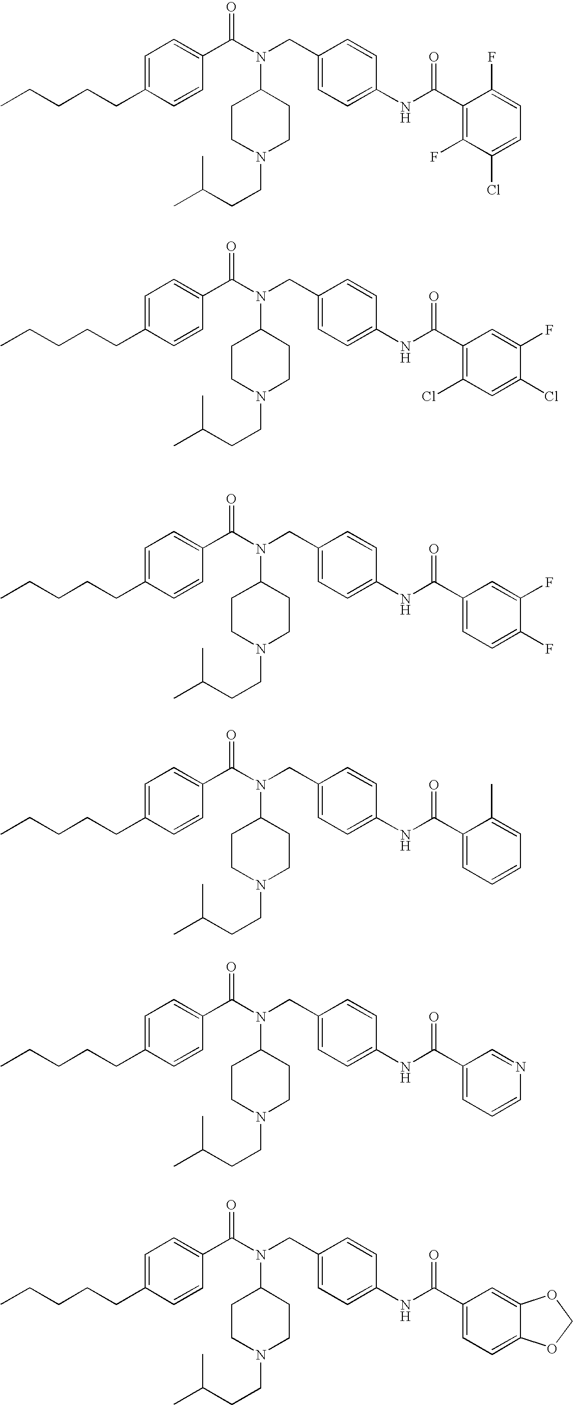

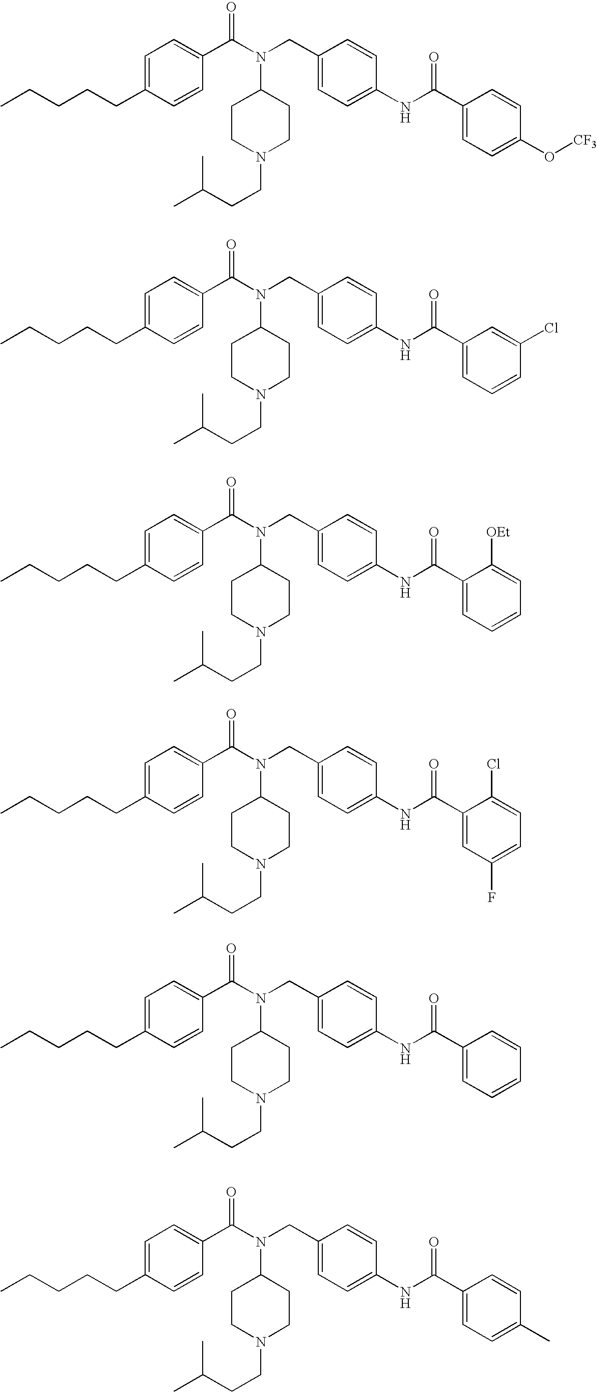

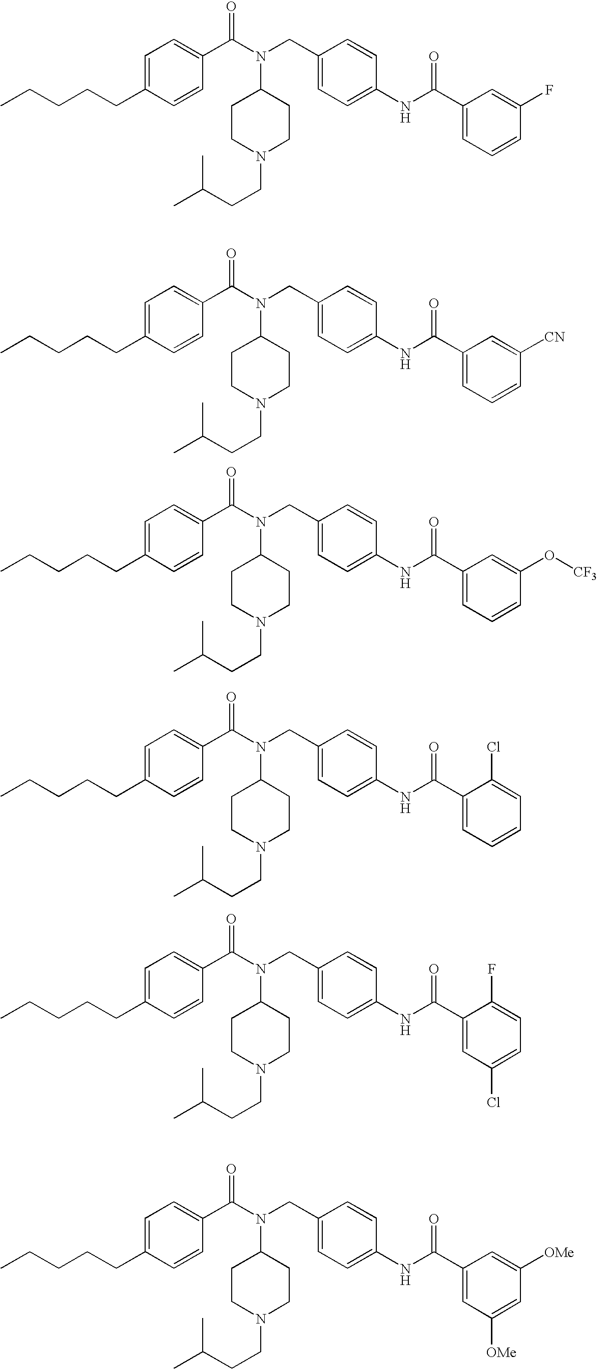

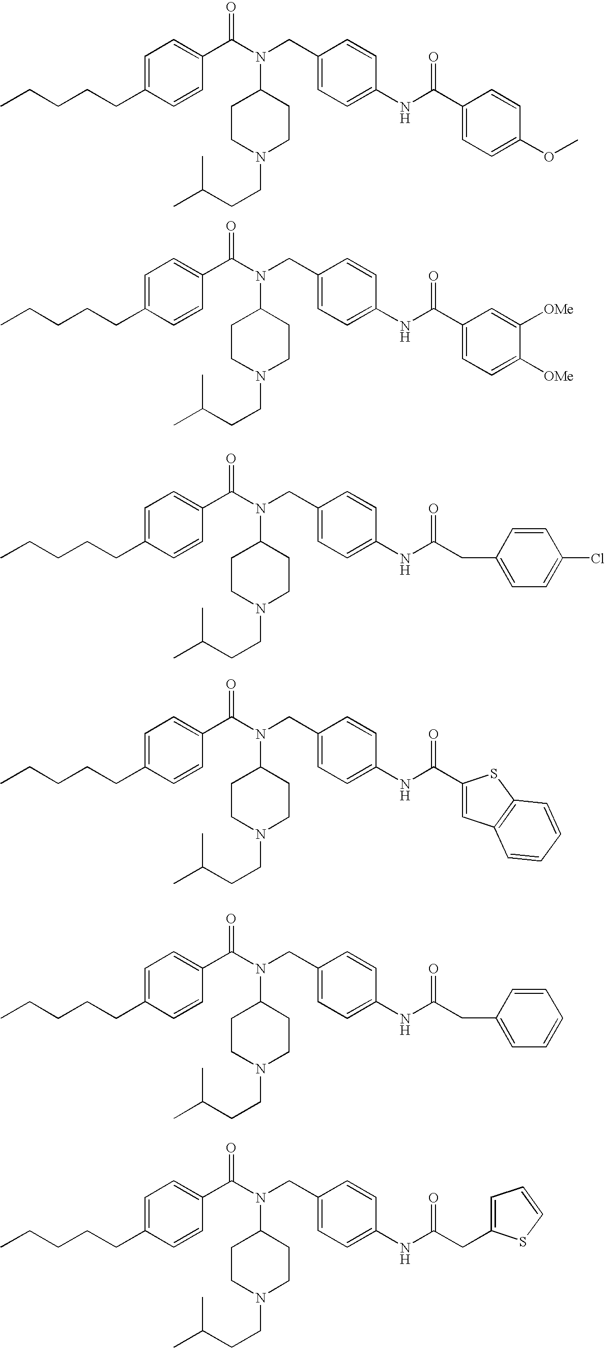

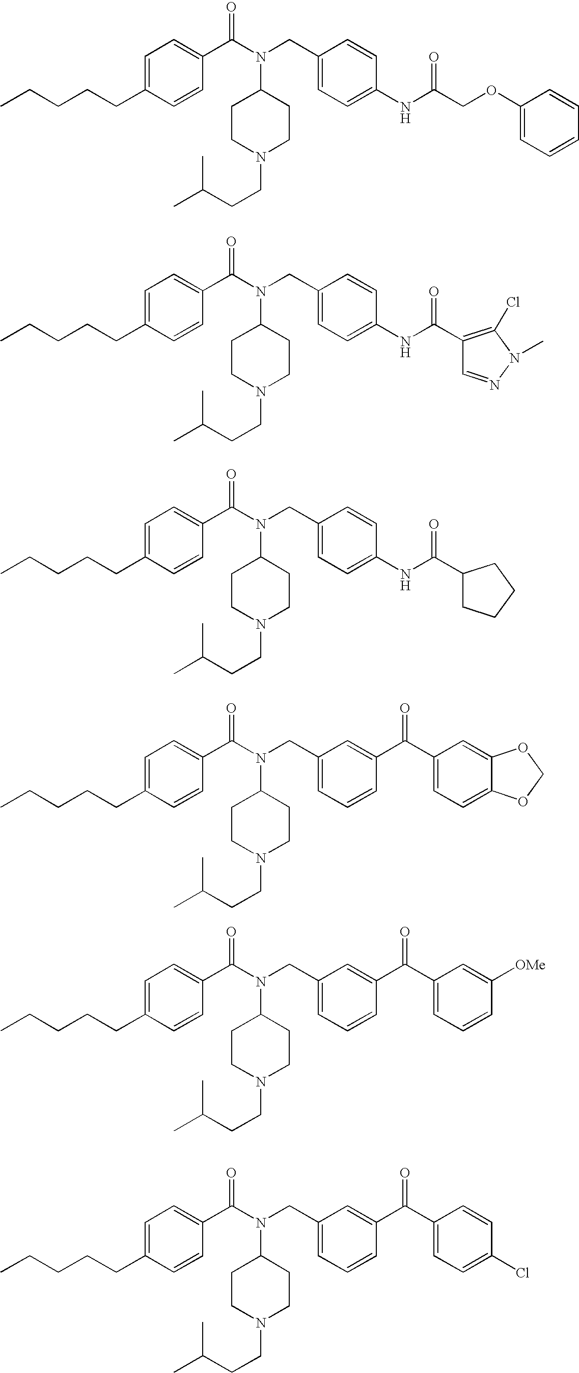

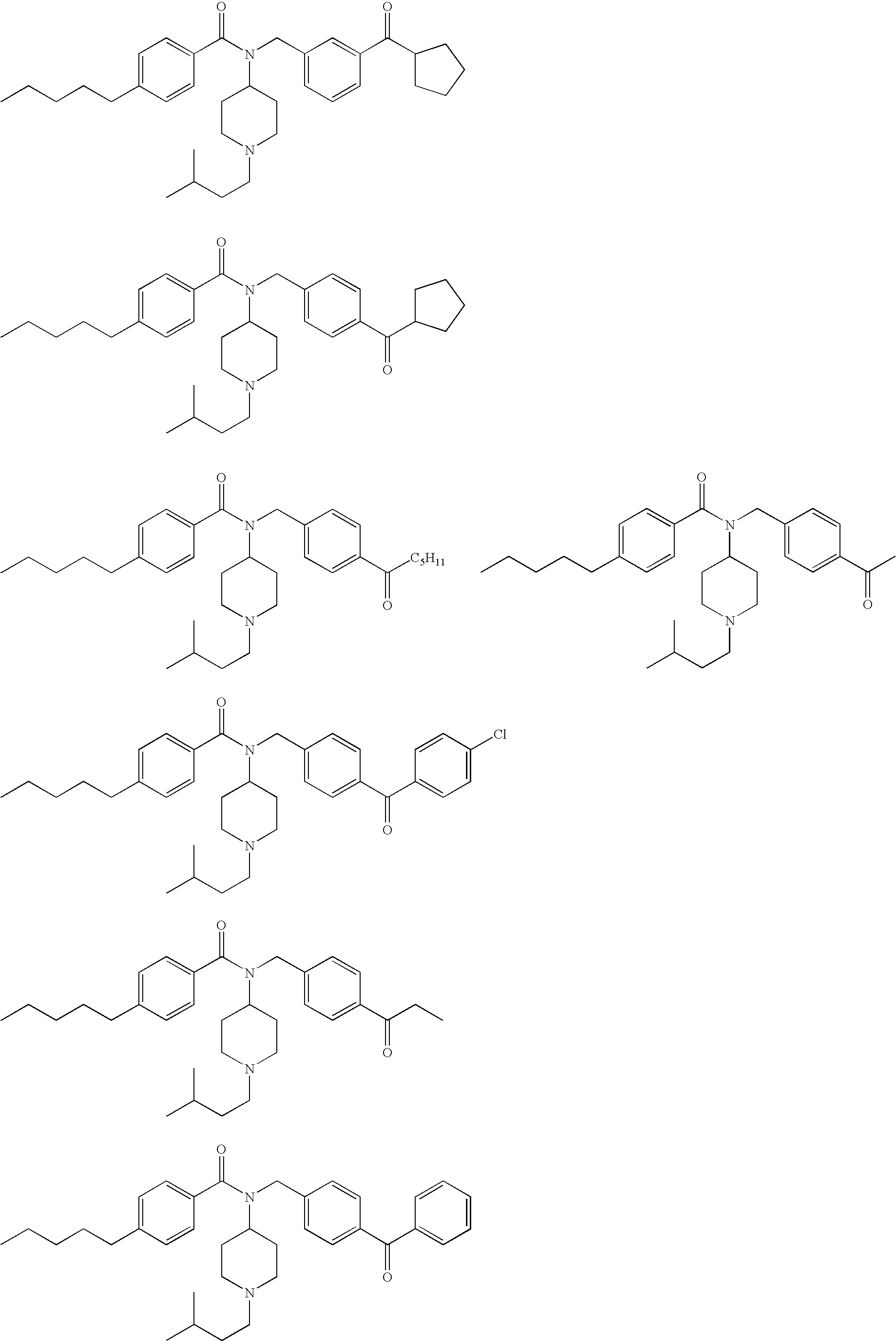

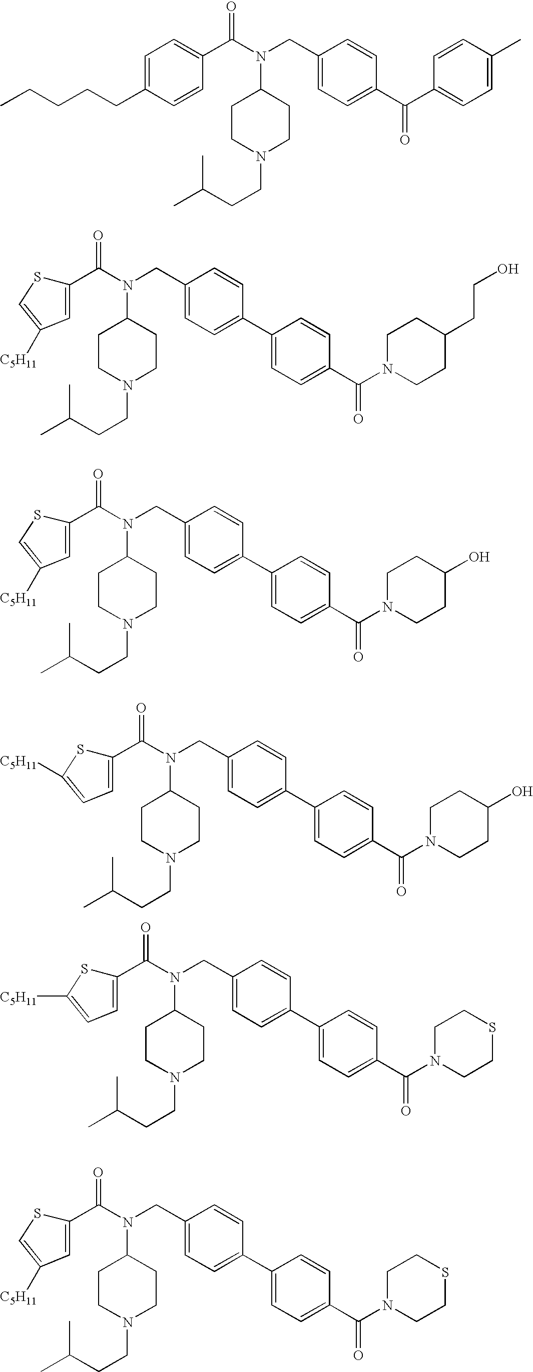

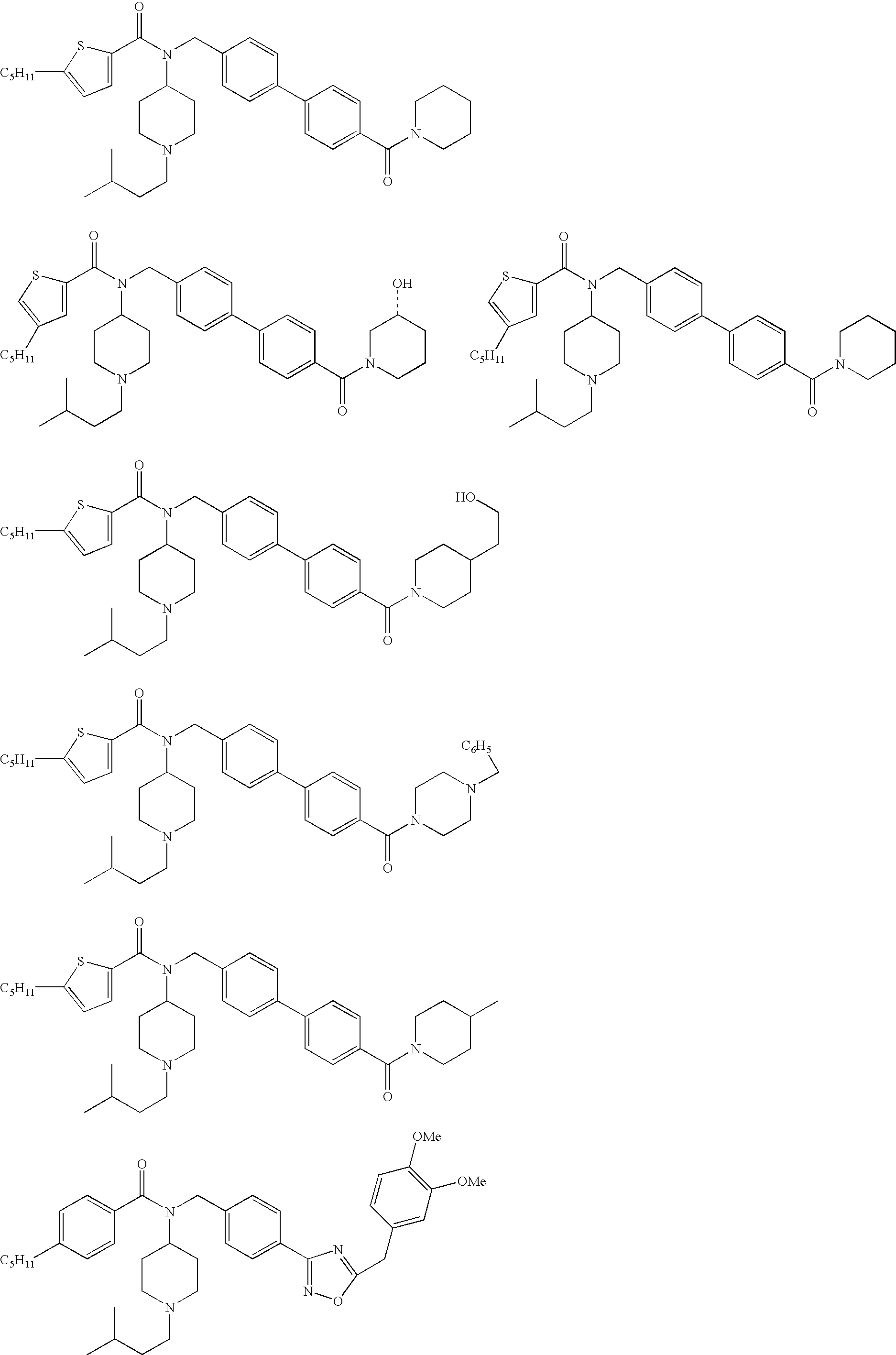

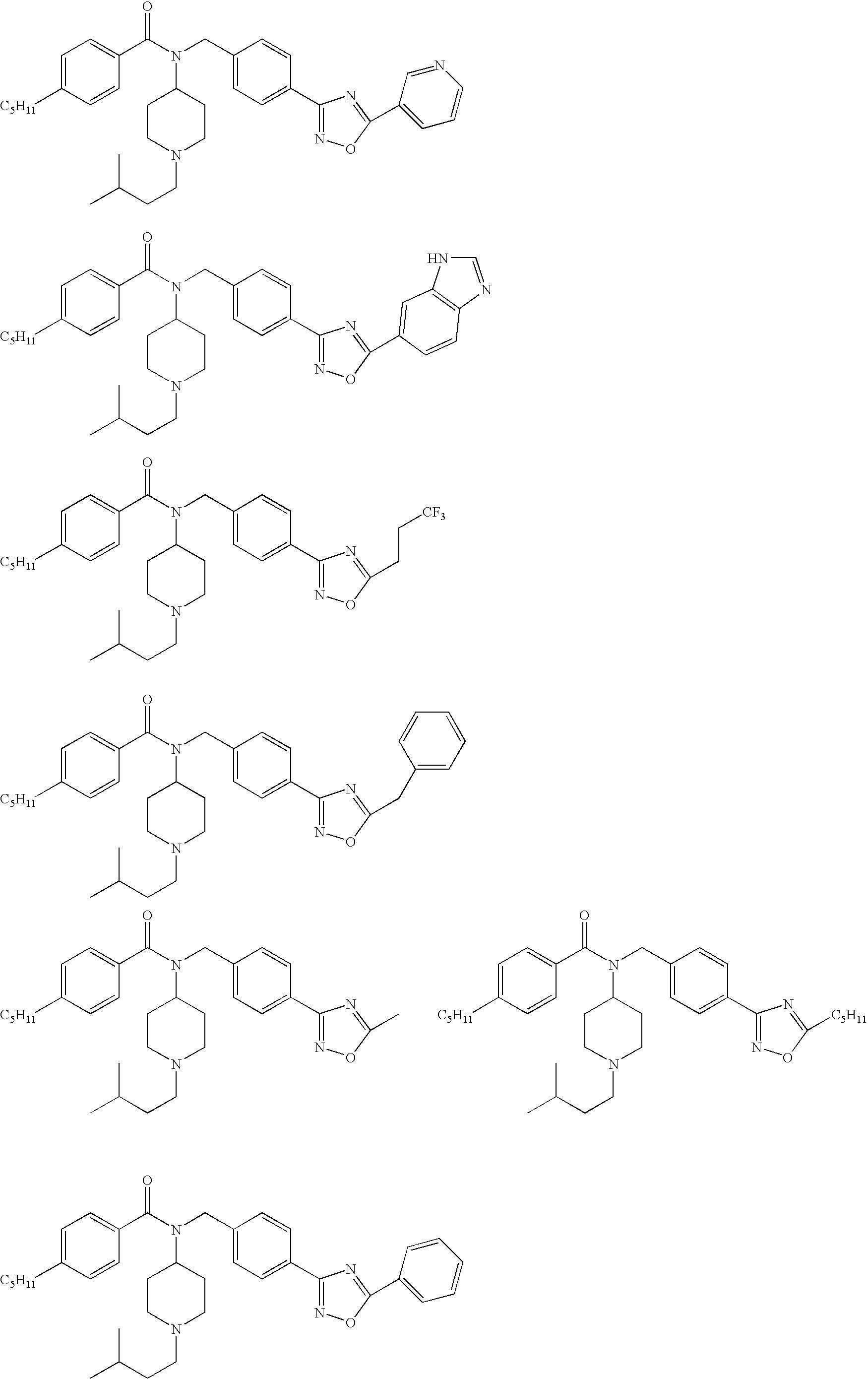

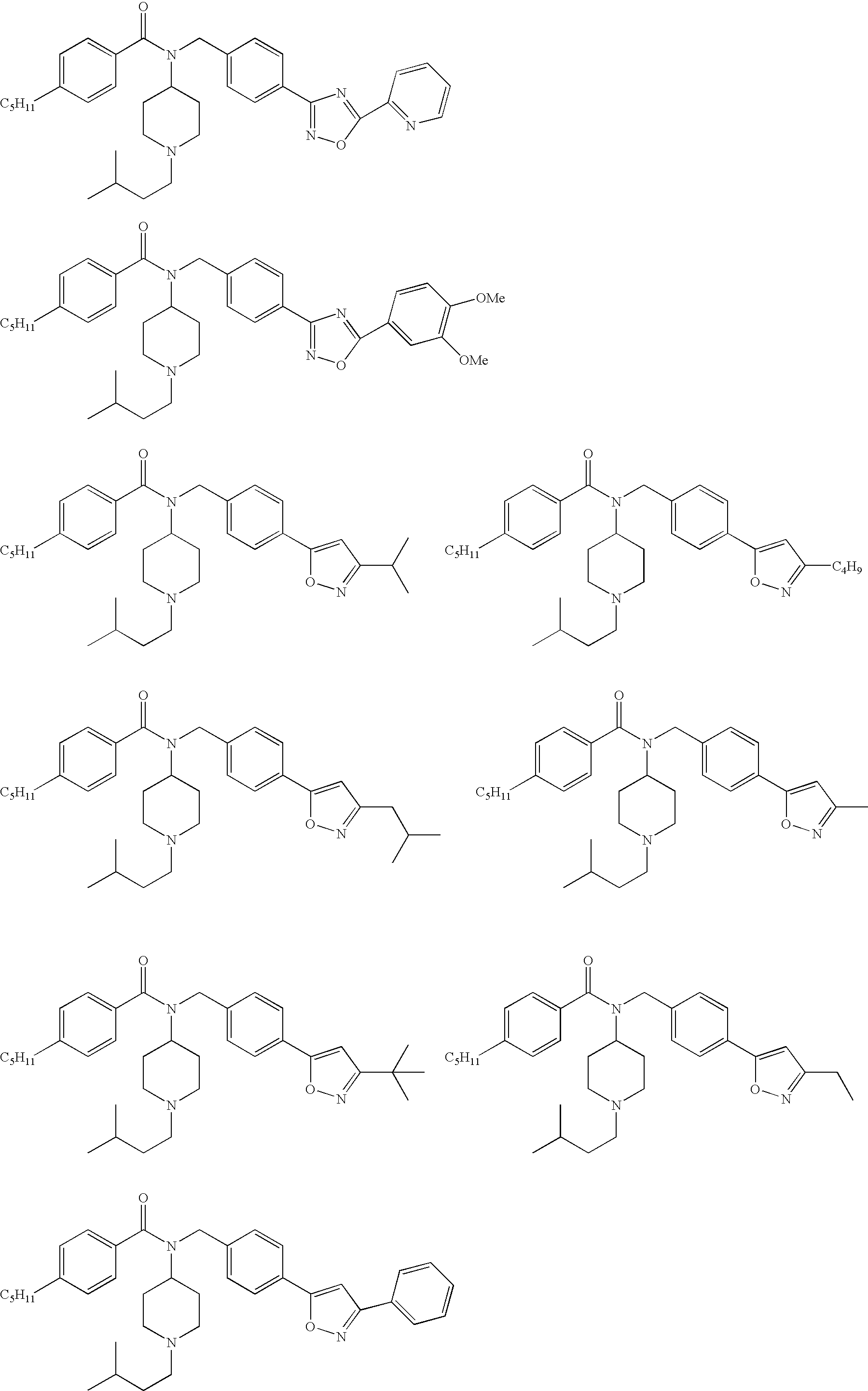

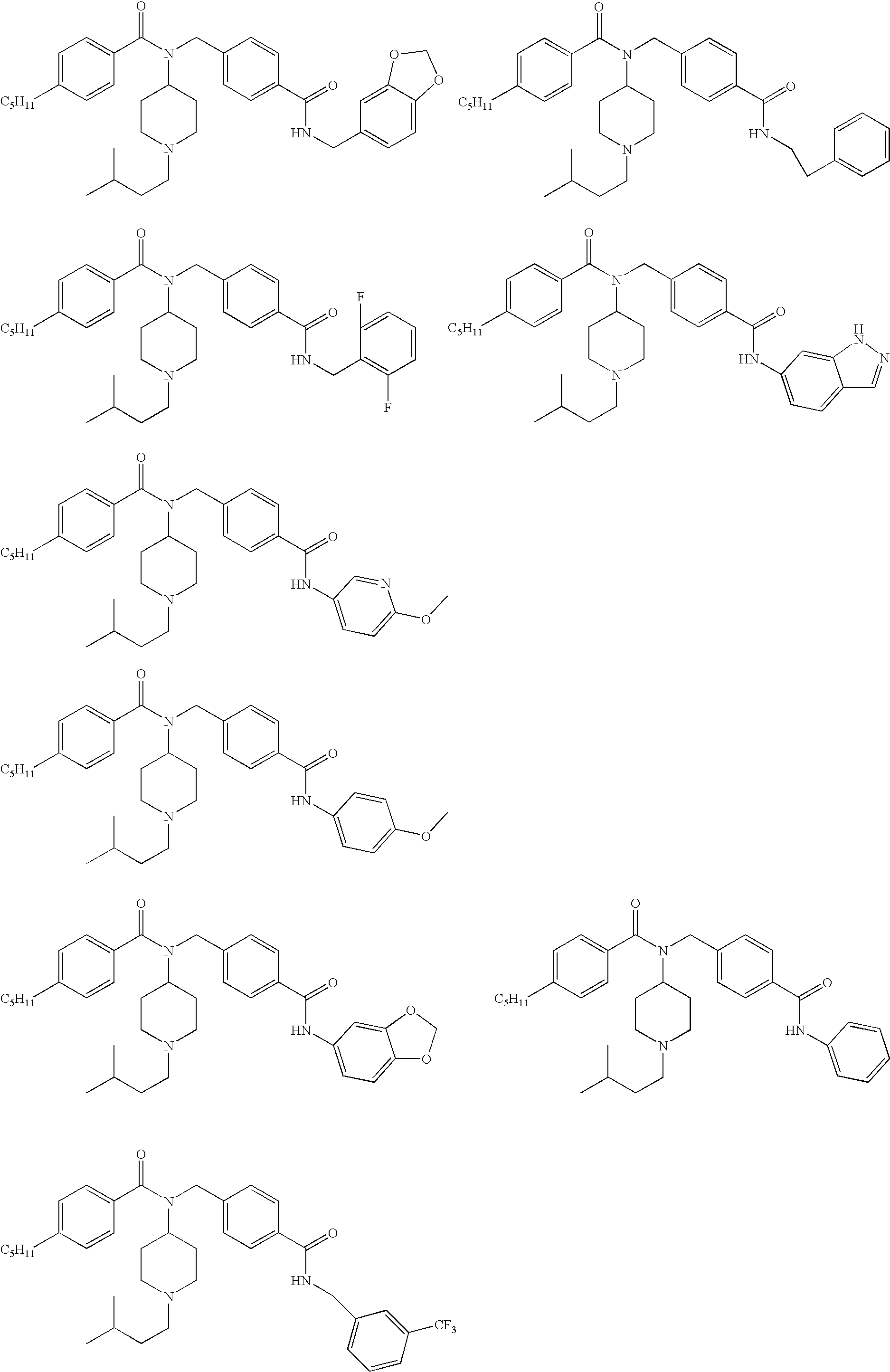

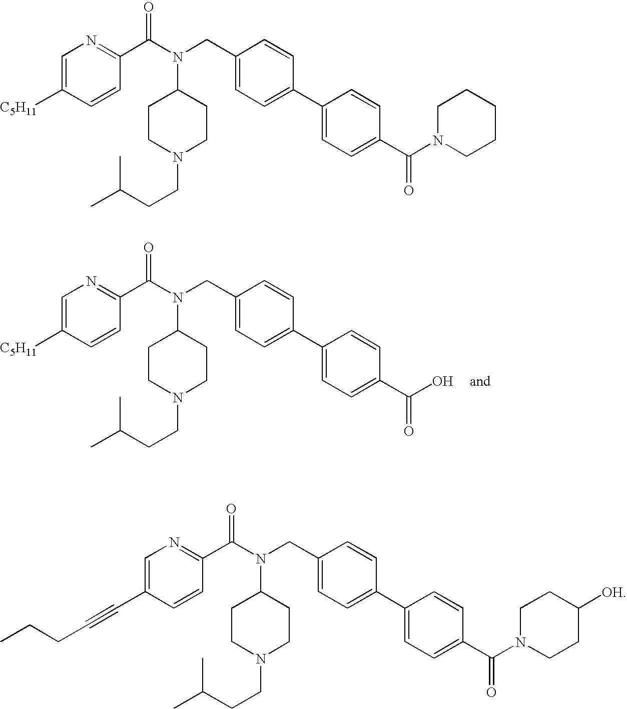

10. A compound according to claim 1 selected from the group consisting of:

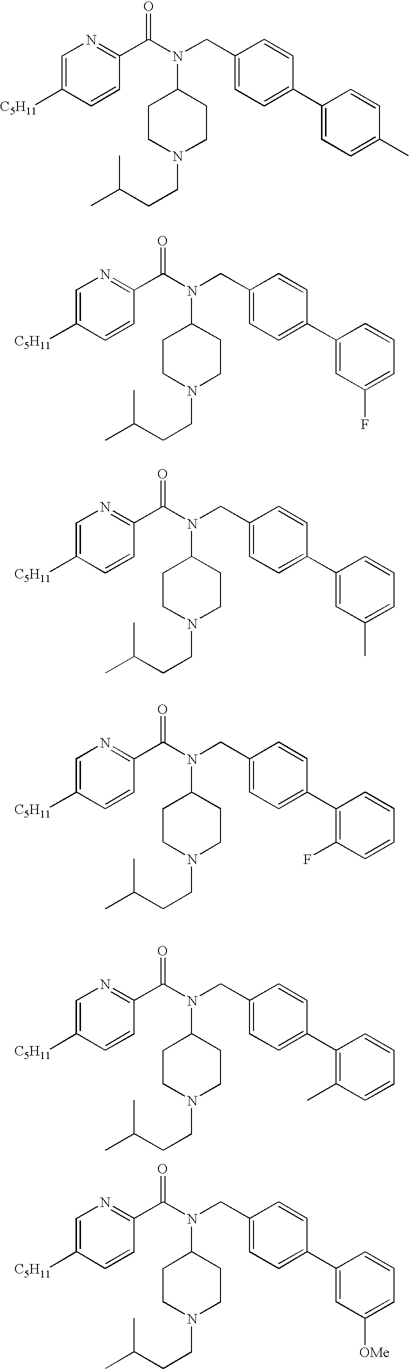

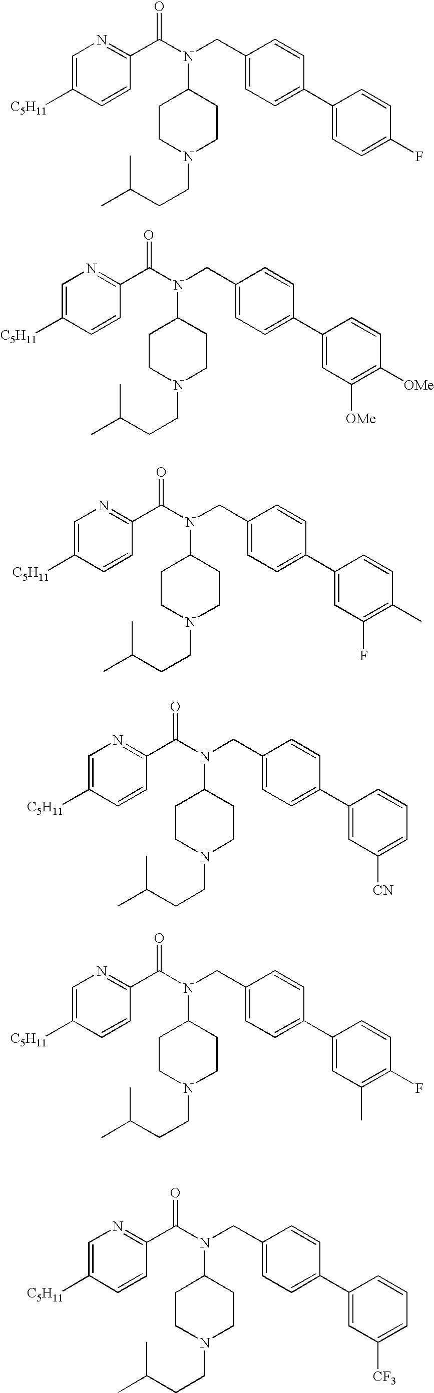

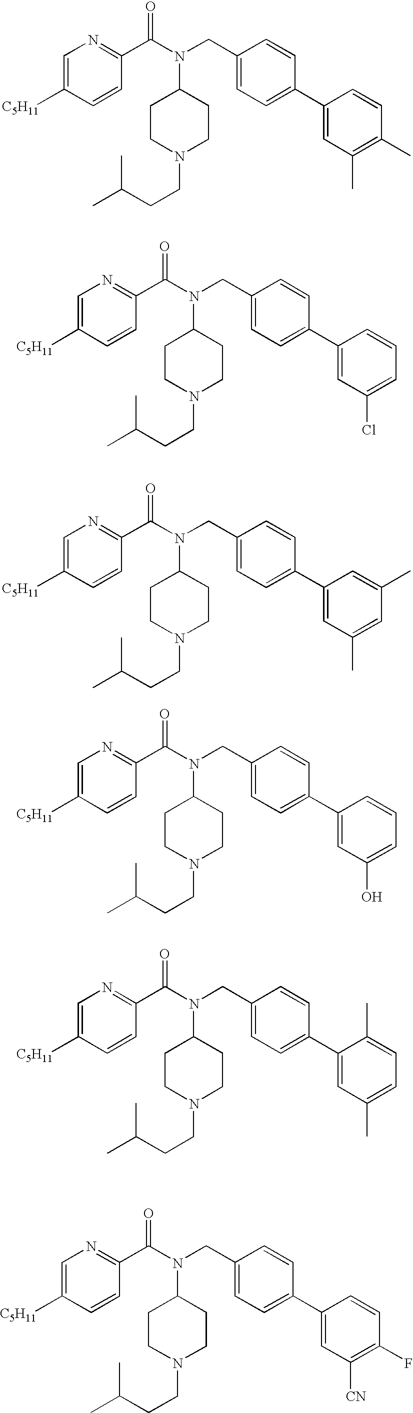

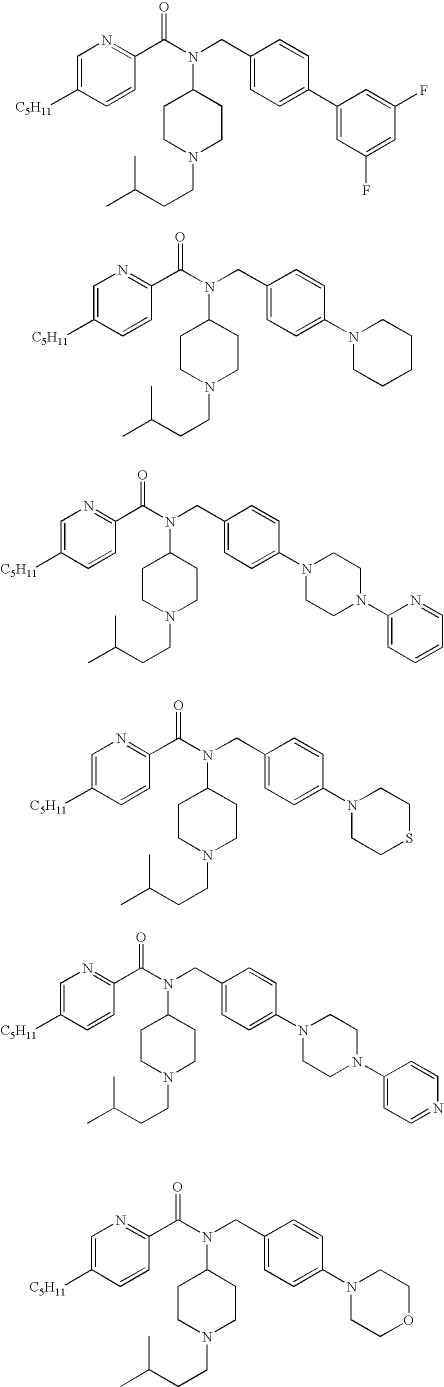

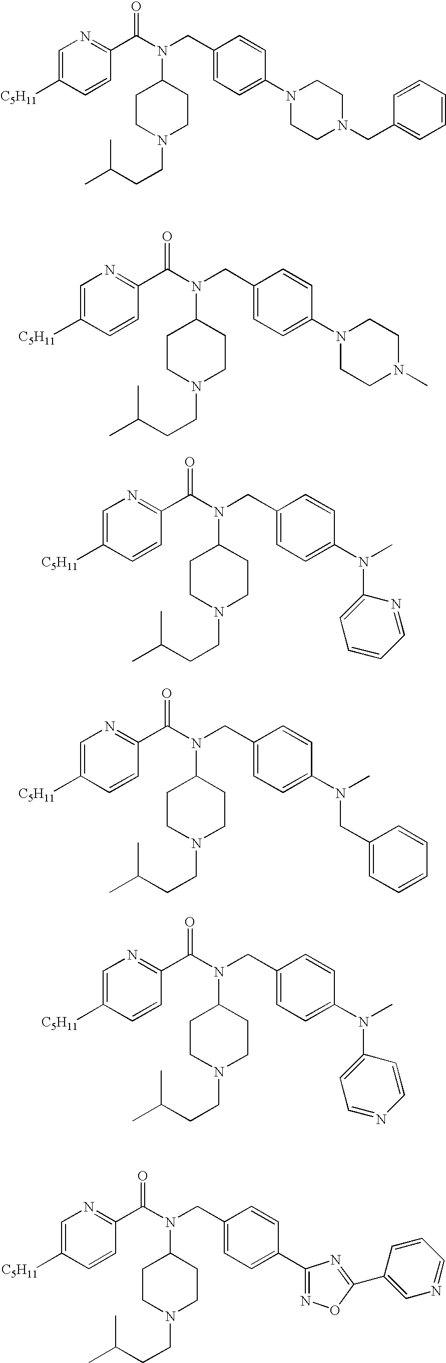

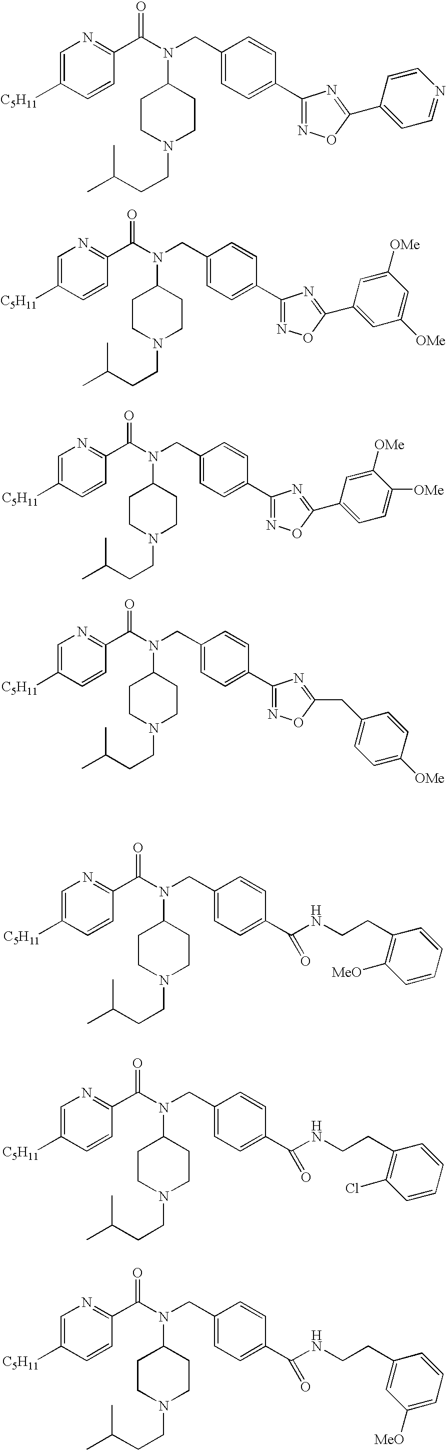

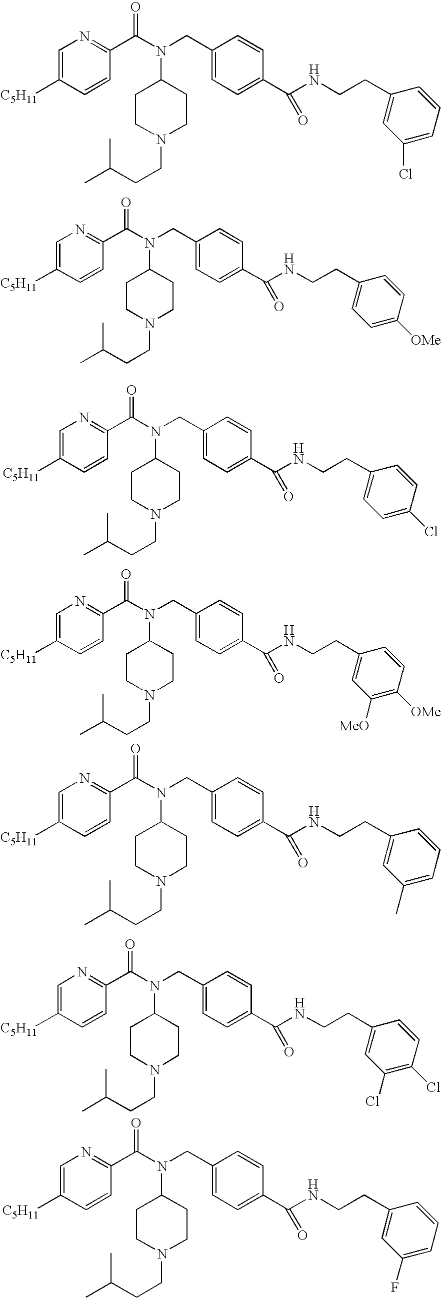

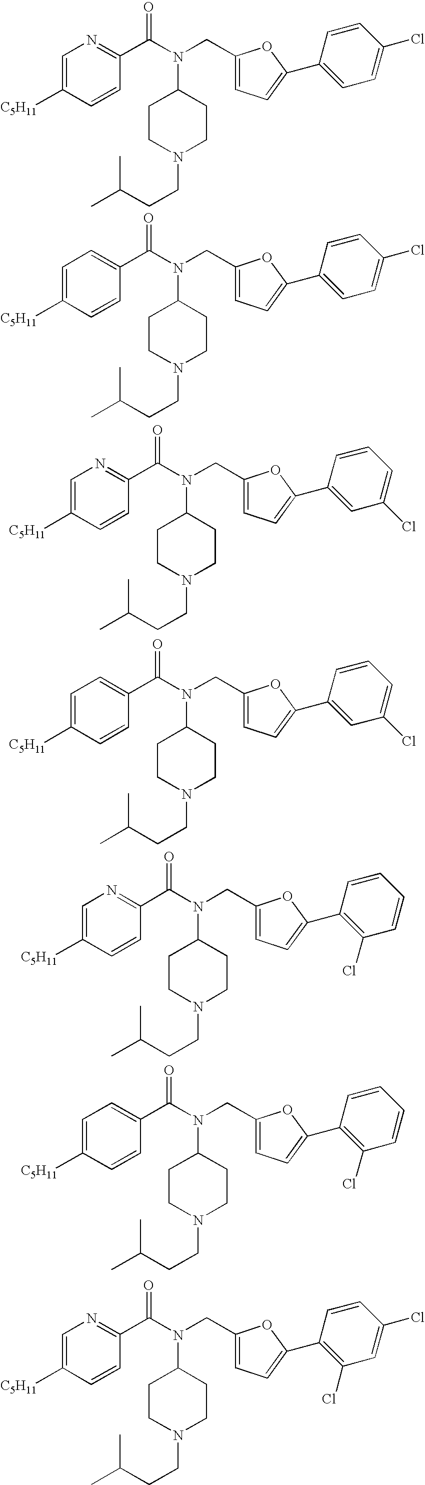

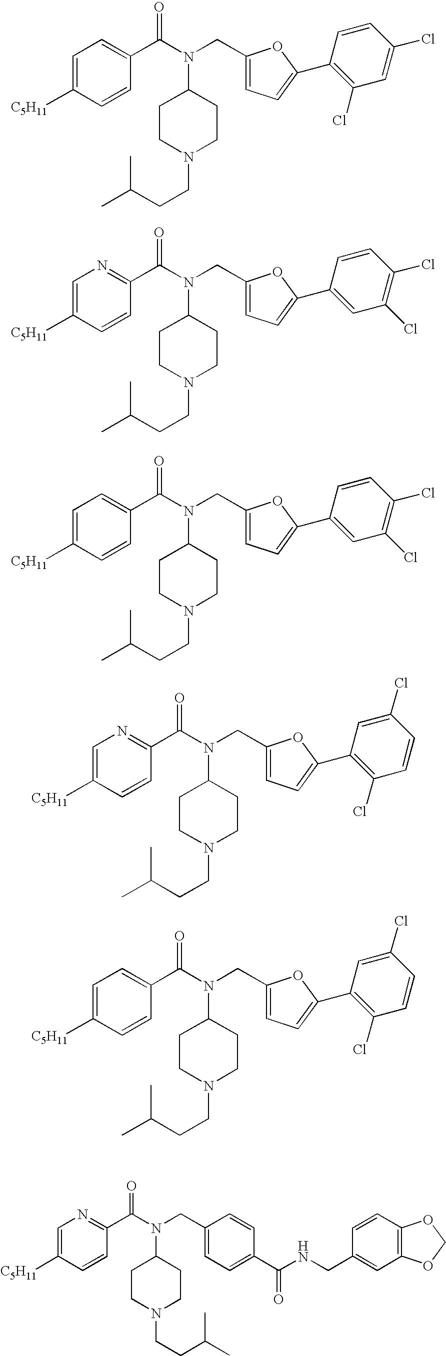

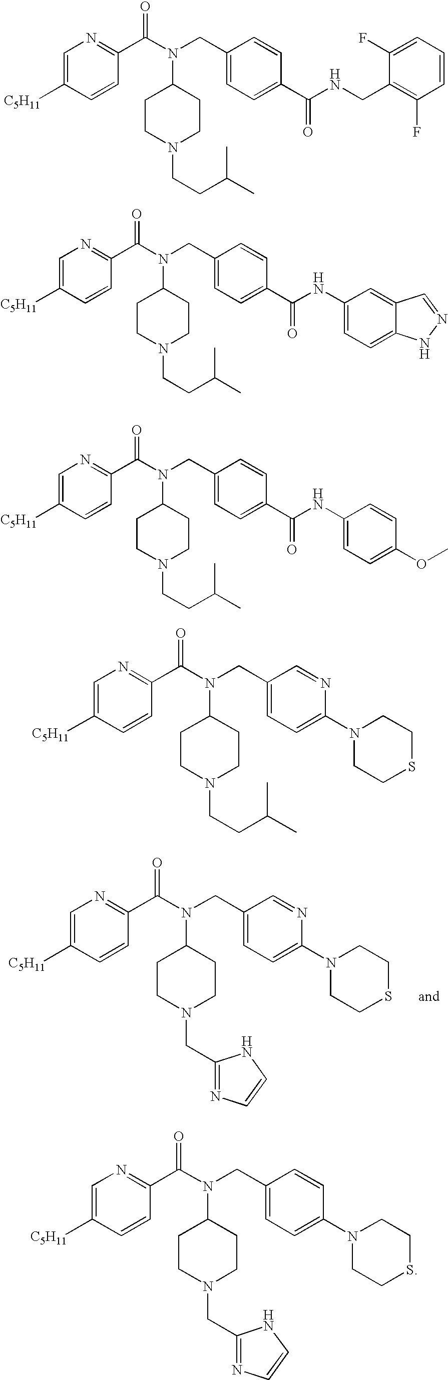

11. A pharmaceutical composition comprising a compound according to any one of claims 1 to 10 and a pharmaceutically acceptable carrier material.

12-14. (canceled)

15. A method of preventing or treating a protozoal infection, comprising administering to a subject in need thereof an effective amount of the pharmaceutical composition of claim 11.

16. The method of claim 15, wherein the protozoal infection is malaria.
